(19) United States
(12) Patent Application Publication
(10) Pub. No.: US 2023/0107538 A1
(43) Pub. Date: Apr. 6, 2023

(54) CYCLOALKYL-CONTAINING CARBOXYLIC ACIDS AND USES THEREOF

(71) Applicant: LIMINAL BIOSCIENCES LIMITED, CAMBRIDGE CAMBRIDGESHIRE (GB)

(72) Inventors: LYNE GAGNON, LAVAL (CA); FRANÇOIS LEBLOND, REPENTIGNY (CA); LILIANNE GEERTS, SAINTE-MARTHE (CA); BOULOS ZACHARIE, LAVAL (CA); CHRIS DOYLE, MONTREAL (CA); JULIEN MARTEL, LAVAL (CA); JEAN-SIMON DUCEPPE, SAINT-JEROME (CA); JEAN-ERIC LACOSTE, LAVAL (CA); JEAN-FRANÇOIS THIBODEAU, OTTAWA (CA)

(21) Appl. No.: 17/783,128

(22) PCT Filed: Dec. 18, 2020
(86) PCT No.: PCT/IB2020/062218

§ 371 Date: Jun. 7, 2022

Related U.S. Application Data
(60) Provisional application No. 62/950,407, filed on Dec. 19, 2019.

(51) Int. Cl. C07C 53/134; C07C 59/205; C07C 61/08; C07D 211/34; C07D 241/04; C07C 57/03; C07C 53/136; C07C 61/04; C07C 59/11; C07C 55/28; C07C 53/132; C07C 57/26; C07D 303/38; A61P 9/12; A61P 19/04; A61P 35/00; A61P 13/12; A61P 7/06

(52) U.S. Cl. CPC C07C 53/134; C07C 59/205; C07C 61/08; C07D 211/34; C07D 241/04; C07C 57/03; C07C 53/136; C07C 61/04; C07C 59/11; C07C 55/28; C07C 53/132; C07C 57/26; C07D 303/38; A61P 9/12; A61P 19/04; A61P 35/00; A61P 13/12; A61P 7/06; C07B 2200/05

(57) ABSTRACT

The present application discloses a compound of formula (I) or a salt thereof: (I) and compositions comprising such compound or salt thereof. The use of such compound, salt thereof or composition comprising same for treating anemia or leukopenia, fibrosis, cancer, hypertension and/or a metabolic condition in a subject is also disclosed.

### CROSS REFERENCE TO RELATED APPLICATIONS

N/A.

### TECHNICAL FIELD

The present disclosure relates to compounds, compositions, methods and uses, such as for the prevention or treatment of various diseases and conditions arising from anemia, neutropenia, leukopenia, inflammation, hypertension, cancer and/or fibrosis in subjects.

### BACKGROUND ART

Hematopoiesis refers to the process of formation, development and differentiation of all types of blood cells. All cellular blood components are derived from hematopoietic stem cells, including leukocytes and erythrocytes. Leukocytes or white blood cells (WBCs) are the cells of the immune system that defend the body against infectious disease and foreign materials. Erythrocytes are the non-nucleated, biconcave, disk-like cells which contain hemoglobin and these cells are essential for the transport of oxygen. A reduction in the number of white blood cells is called leukopenia whereas anemia refers to the condition in which there is a reduction below normal levels in the number of erythrocytes, the quantity of hemoglobin, or the volume of packed red blood cells in the blood. Disorders of the blood and several kinds of leukopenia and anemia may be produced by a variety of underlying causes, including chemotherapy (e.g., chemotherapy-induced anemia) and cancers (e.g., cancer-related anemia). Therefore, there is a need for novel compositions and methods to stimulate hematopoiesis and to address the undesirable side effects of myelosuppression induced by chemotherapy and radiation therapy.

Immune Mediated Inflammatory Disease (IMID) refers to any of a group of conditions or diseases that lack a definitive etiology but which are characterized by common inflammatory pathways leading to inflammation, and which may result from, or be triggered by, a dysregulation of the normal immune response. Autoimmune disease refers to any of a group of diseases or disorders in which tissue injury is associated with a humoral and/or cell-mediated immune response to body constituents or, in a broader sense, an immune response to self. Current treatments for autoimmune disease can be broadly classified into two groups: those drugs which dampen or suppress the immune response to self and those drugs which address the symptoms that arise from chronic inflammation. Conventional treatments for autoimmune diseases (e.g., primarily arthritis) are (1) Nonsteroidal Anti-Inflammatory Drugs (NSAIDs) such as aspirin, ibuprofen, naproxen, etodolac, and ketoprofen; (2) Corticosteroids such as prednisone and dexamethasone; (3) Disease-Modifying Anti-Rheumatic Drugs (DMARDs) such as methotrexate, azathioprine, cyclophosphamide, cyclosporin A, Sandimmune™, Neoral™, FK506 (tacrolimus)™; and JAK-1 inhibitors (Filgotinib); (4) Biologicals such as the recombinant proteins Remicade™, Enbrel™ and Humira™. While numerous therapies are available, conventional treatments are not routinely efficacious. More problematic is the accompanying toxicity which often prohibits the long-term use necessary for treatment of a chronic disease. Therefore, there is a need for compounds that are useful for the treatment of inflammation-related diseases, including chronic and non-chronic autoimmune disease.

Fibrosis refers to the formation or development of excess fibrous connective tissue in an organ or tissue that can occur as a part of the wound-healing process in damaged tissue. It may be viewed as an exaggerated form of wound healing that does not resolve itself. Fibrosis can occur on the skin but it can also occur in internal organs such as the kidney, heart, lung, liver, gut, pancreas, urinary tract, bone marrow and brain. In the case of organs, fibrosis will often precede sclerosis and subsequent shutdown of the affected organ. Of course, the most common consequence of complete organ failure is death. Thus, for example, pulmonary fibrosis is a major cause of morbidity and mortality. It is associated with the use of high dose chemotherapy (e.g., bleomycin) and bone marrow transplantation. Idiopathic pulmonary fibrosis (IPF) is a lung fibrotic disease for which the median survival is four to five years after the onset of symptoms. Currently there are no effective antifibrotic drugs approved for human needs. Therefore, the need exists for compounds that are useful for the treatment of fibrotic diseases.

Hypertension, also known as high blood pressure, is a long-term medical condition in which the blood pressure in the arteries is persistently elevated. High blood pressure typically does not cause symptoms. Long-term high blood pressure, however, is a major risk factor for coronary artery disease, stroke, heart failure, atrial fibrillation, peripheral arterial disease, vision loss, chronic kidney disease, and dementia. Hypertension is classified as either primary (essential) high blood pressure or secondary high blood pressure. About 90-95% of cases are primary, defined as high blood pressure due to nonspecific lifestyle and genetic factors. Lifestyle factors that increase the risk include excess salt in the diet, excess body weight, smoking, and alcohol use. The remaining 5-10% of cases are categorized as secondary high blood pressure, defined as high blood pressure due to an identifiable cause, such as chronic kidney disease, narrowing of the kidney arteries, an endocrine disorder, or the use of birth control pills. Secondary hypertension results from an identifiable cause. Kidney disease is the most common secondary cause of hypertension. Hypertension can also be caused by endocrine conditions, such as Cushing's syndrome, hyperthyroidism, hypothyroidism, acromegaly, Conn's syndrome or hyperaldosteronism, renal artery stenosis (from atherosclerosis or fibromuscular dysplasia), hyperparathyroidism, and pheochromocytoma. Other causes of secondary hypertension include obesity, sleep apnea, pregnancy, coarctation of the aorta, excessive eating of liquorice, excessive drinking of alcohol, certain prescription medicines, herbal remedies, and stimulants such as cocaine and methamphetamine. Arsenic exposure through drinking water has been shown to correlate with elevated blood pressure. Depression was also linked to hypertension. Several classes of medications, collectively referred to as antihypertensive medications, are available for treating hypertension.

First-line medications for hypertension include thiazide-diuretics, calcium channel blockers, angiotensin converting enzyme inhibitors (ACE inhibitors), and angiotensin receptor blockers (ARBs). These medications may be used alone or in combination (ACE inhibitors and ARBs are not recommended for use in combination); the latter option may serve to minimize counter-regulatory mechanisms that act to restore blood pressure values to pre-treatment levels. Most people require more than one medication to control their hypertension. Therefore, there is a need for alternative therapies for the treatment of hypertension.

Cancer refers to more than one hundred clinically distinct forms of the disease. Almost every tissue of the body can give rise to cancer and some can even yield several types of cancer. Cancer is characterized by an abnormal growth of cells which can invade the tissue of origin or spread to other sites. In fact, the seriousness of a particular cancer, or the degree of malignancy, is based upon the propensity of cancer cells for invasion and the ability to spread. That is, various human cancers (e.g., carcinomas) differ appreciably as to their ability to spread from a primary site or tumor and metastasize throughout the body.

The twelve major cancers are prostate, breast, lung, colorectal, bladder, non-Hodgkin's lymphoma, uterine, melanoma, kidney, leukemia, ovarian, and pancreatic cancers. Generally, four types of treatment have been used for the treatment of metastatic cancers: surgery, radiation therapy, chemotherapy, and immunotherapy. Surgery may be used to remove the primary tumor and/or to improve the quality of life by removing a metastasis, for example, that is obstructing the gastrointestinal tract. Radiation therapy may also be used for treatment of a primary tumor where it is difficult to surgically remove the entire tumor and/or to treat cutaneous and/or lymph node metastasis. A number of chemotherapeutic drugs are available for the treatment of cancer and most often the treatment regimen calls for a combination of these drugs, primarily to deal with the phenomena of drug resistance. That is, the biochemical process which develops over time whereby the cancer is no longer responsive, or becomes refractory, to a particular chemotherapeutic drug prior to eradication of the cancer. These treatments have also met with limited success. Therefore, a need still exists for novel compounds for the treatment of cancers.

Diabetes is caused by multiple factors and is characterized by elevated levels of plasma glucose (hyperglycemia) in the fasting state. There are two generally recognized forms of diabetes: Type I diabetes, or insulin dependent diabetes, in which patients produce little or no insulin and Type II diabetes, or noninsulin-dependent diabetes wherein patients produce insulin, while at the same time demonstrating hyperglycemia. Type I diabetes is typically treated with exogenous insulin administered via injection. However, Type II diabetics often present “insulin resistance”, such that the effect of insulin in stimulating glucose and lipid metabolism in the main insulin-sensitive tissues, namely muscle, liver and adipose tissues, is diminished and hyperglycemia results.

Persistent or uncontrolled hyperglycemia that occurs in diabetes is associated with increased morbidity and premature mortality. Abnormal glucose homeostasis is also associated, both directly and indirectly, with obesity, hypertension and alterations in lipid, lipoprotein and apolipoprotein metabolism. Type II diabetics are at increased risk of cardiovascular complications such as atherosclerosis, coronary heart disease, stroke, peripheral vascular disease, hypertension, nephropathy, retinopathy and also neuropathy. Many patients who have insulin resistance, but have not developed Type II diabetes, are also at risk of developing symptoms referred to as “Syndrome X”, or “Metabolic Syndrome”. Metabolic syndrome is characterized by insulin resistance, along with abdominal obesity, hyperinsulinemia, high blood pressure, low HDL (high density lipoproteins) and high VLDL (very low density lipoprotein), hypertriglyceridemia and hyperuricemia. Whether or not they develop overt diabetes, these patients are at increased risk of developing cardiovascular complications.

Current treatments for diabetes include: insulin, insulin secretagogues, such as sulphonylureas, which increase insulin production from pancreatic s-cells; glucose-lowering effectors, such as metformin which reduce glucose production from the liver; activators of the peroxisome proliferator-activated receptor-γ (PPAR-γ), such as the thiazolidinediones, which enhance insulin action; dipeptidyl peptidase-4 (DPP-4) inhibitors which inhibit the degradation of GLP-1 and α-glucuronidase inhibitors which interfere with gut glucose production. However, there are some deficiencies associated with these treatments. For example, sulphonylureas and insulin injections can be associated with hypoglycemia and weight gain. Responsiveness to sulphonylureas is often lost over time. An increased relative risk of pancreatic cancer, and to a lesser extent other neoplasms, has been linked to the use of DPP-4 inhibitors. Gastrointestinal problems are observed with metformin and α-glucosidase inhibitors. Finally, PPAR-γ agonists may cause increase weight and edema.

The present description refers to a number of documents, the content of which is herein incorporated by reference in their entirety.

### SUMMARY OF THE DISCLOSURE

The present disclosure relates to compounds, compositions, methods and uses, such as for the prevention or treatment of various diseases and conditions arising from anemia, neutropenia, leukopenia, inflammation, hypertension, cancer, metabolic conditions and/or fibrosis in subjects.

In aspects and embodiments, the present disclosure relates to the following items:
1. A compound of formula (I) or a salt thereof:

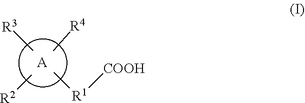

wherein:
A represents a 3- to 6-membered cycloalkane or heterocycloalkane, wherein the cycloalkane or heterocycloalkane are optionally bridged,
R¹ represents a covalent bond or an alkylene or alkenylene chain, wherein the alkylene or alkenylene chain is optionally substituted with ═O,
R² represents a hydrogen atom or an alkyl or alkenyl chain, wherein:
   the alkyl or alkenyl chain is optionally substituted with a hydroxy group, or
   the alkyl or alkenyl chain is optionally terminated with a carboxyl group or with a 3- to 6-membered cycloalkyl, heterocycloalkyl, aryl or heteroaryl, and
   the cycloalkyl, heterocycloalkyl, aryl, and heteroaryl are optimally substituted with one or more alkyl groups, and
R³ and R⁴ are identical to each other or different, are both attached to a same ring atom of A, and represent hydrogen atoms, deuterium atoms, halogen atoms, or methyl groups, or
R³ represents R², wherein R² is as defined above, and R⁴ represents a hydrogen atom,
R¹ and R² are attached on a same ring atom of A or on different ring atoms of A,
   wherein the atom of R¹, or of A if R¹ is a covalent bond, that bears the —COOH group is optionally substituted with a second —COOH group,
   wherein A, R¹ and R² are such that the shortest continuous chain of carbon atoms and, if present, heteroatoms linking:
the carbon atom or ring heteroatom in R² that is farthest from R¹ or, if R² represents a hydrogen atom, the ring carbon atom or ring heteroatom in A that is farthest from R¹;
to the carbon atom of the COOH group terminating R¹,
   is 9 to 11 atoms long
   wherein the COOH group may be replaced by an isostere thereof;
   and wherein the compound is not

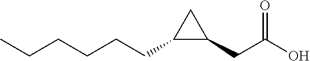

(cascarillic acid) or

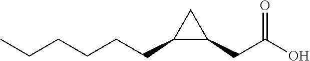

(cis-2-(2-hexylcyclopropyl)-acetic acid).
2. The compound or salt thereof according to item 1, wherein A represents a 3- to 6-membered cycloalkane.
3. The compound or salt thereof according to item 2, wherein the 3- to 6-membered cycloalkane is cyclopropane.
4. The compound or salt thereof according to item 1, wherein the heterocycloalkane is ethylene oxide, piperidine or piperazine.
5. The compound or salt thereof according to item 1, wherein the cycloalkane or heterocycloalkane in A is bridged.
6. The compound or salt thereof according to item 5, wherein the bridged cycloalkane or heterocycloalkane is bicyclo[2.2.2]octane.
7. The compound or salt thereof according to any one of items 1 to 6, wherein R¹ and R² are attached on a same ring atom of the cycloalkane or heterocycloalkane in A.
8. The compound or salt thereof according to any one of items 1 to 6, wherein R¹ and R² are attached on different ring atoms of the cycloalkane or heterocycloalkane in A.
9. The compound or salt thereof according to any one of items 1 to 8, wherein A represents:
   cyclopropane with R¹ and R² attached on a same atom of the cyclopropane,
   cyclopropane with R¹ and R² attached on adjacent atoms of the cyclopropane,
   ethylene oxide with R¹ and R² attached on adjacent ring atoms of the ethylene oxide,
   cyclobutane with R¹ and R² attached on a same ring atom of the cyclobutane,
   cyclobutane with R¹ and R² attached on adjacent ring atoms of the cyclobutane,
   cyclobutane with R¹ and R² attached on opposite ring atoms of the cyclobutane,
   cyclohexane with R¹ and R² attached on opposite ring atoms of the cyclohexane,
   cyclohexane with R¹ and R² attached on ring atoms of the cyclohexane that are separated by a single other ring atom,
   piperidine with R¹ and R² attached on opposite ring atoms of the piperidine,
   piperazine with R¹ and R² attached on ring atoms of the piperazine that are separated by a single other ring atom, or
   bicyclo[2.2.2]octane with R¹ and R² attached on opposite ring atoms of the bicyclo[2.2.2]octane.
10. The compound or salt thereof according to any one of items 1 to 9, wherein R¹ represents a covalent bond or an alkylene chain.
11. The compound or salt thereof according to any one of items 1 to 10, wherein the alkylene or alkenylene chain in R¹ is a C₁-C₈ chain.
12. The compound or salt thereof according to any one of items 1 to 11, wherein the alkylene or alkenylene chain in R¹ is substituted with ═O.
13. The compound or salt thereof according to any one of items 1 to 11, wherein the alkylene or alkenylene chain in R¹ is unsubstituted.
14. The compound or salt thereof according to any one of items 1 to 13, wherein R² represents a hydrogen atom.
15. The compound or salt thereof according to any one of items 1 to 13, wherein R² represents an alkyl or alkenyl chain.
16. The compound or salt thereof according to item 15, wherein the alkyl or alkenyl chain in R² is a C₁-C₈ chain.
17. The compound or salt thereof according to item 16, wherein the alkyl or alkenyl chain in R² is a C₅-C₇ chain.
18. The compound or salt thereof according to any one of items 1 to 17, wherein the alkyl or alkenyl chain in R² is terminated with a carboxyl group.
19. The compound or salt thereof according to any one of items 1 to 17, wherein the alkyl or alkenyl chain in R² is terminated with hydrogen atoms only.
20. The compound or salt thereof according to any one of items 1 to 17, wherein the alkyl or alkenyl chain in R² is terminated with a 3- to 6-membered cycloalkyl, heterocycloalkyl, aryl or heteroaryl.
21. The compound or salt thereof according to item 20, wherein the cycloalkyl, heterocycloalkyl, aryl or heteroaryl is substituted with one or more alkyl groups.
22. The compound or salt thereof according to item 21, wherein the cycloalkyl, heterocycloalkyl, aryl or heteroaryl is substituted with two alkyl groups.
23. The compound or salt thereof according to item 22, wherein the two alkyl groups are on the same atoms of the cycloalkyl, heterocycloalkyl, aryl or heteroaryl.
24. The compound or salt thereof according to any one of items 20-23, wherein the cycloalkyl, heterocycloalkyl, aryl or heteroaryl terminating the alkyl or alkenyl in R² is:
   cyclopropyl substituted with two identical alkyl groups on the same ring atom,
   cyclohexyl unsubstituted, or
   phenyl substituted with one alkyl group.
25. The compound or salt thereof according to any one of items 1 to 24, wherein R³ and R⁴ are identical to each other.
26. The compound or salt thereof according to any one of items 1 to 25, wherein R³ and R⁴ are both attached to a same ring atom of A.
27. The compound or salt thereof according to any one of items 1 to 24, wherein R³ represents R², and R⁴ represents hydrogen.
28. The compound or salt thereof according to any one of items 1 to 27, wherein the atom of R¹ that bears the —COOH group is optionally substituted with a second —COOH group.
29. The compound or salt thereof according to any one of items 1 to 28, wherein the compound or salt thereof is one of the compounds depicted in Table 1, or a salt thereof:

TABLE 1

| Cmpd # | Structure |
|---|---|
| I | 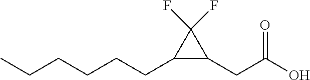 |
| II | 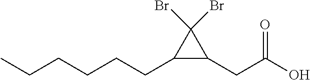 |
| III | 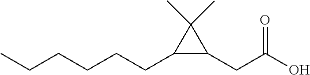 |
| IV | 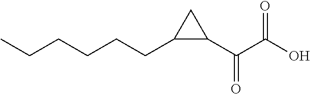 |
| V | 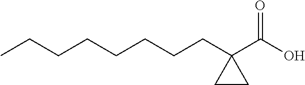 |
| VI | 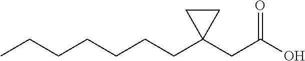 |
| VII | 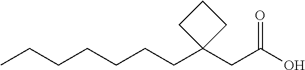 |
| VIII | 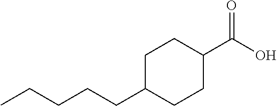 |
| IX | 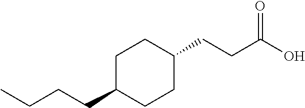 |
| X | 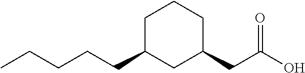 |
| XI | 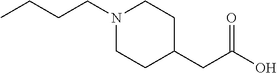 |
| XII | 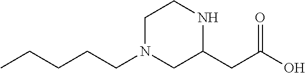 |
| XIII | 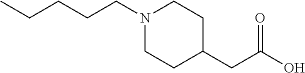 |
| XIV | 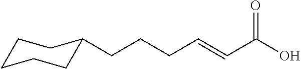 [SMILES: O=C(O)/C=C/CCCC1CCCCC1] |
| XV | 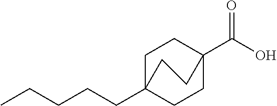 [SMILES: CCCCCC12CCC(C(=O)O)(CC1)CC2] |
| XVI | 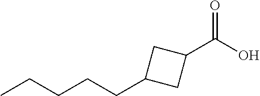 |
| XVII | 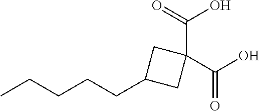 |
| XVIII | 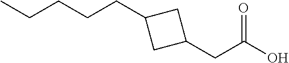 |
| XIX | 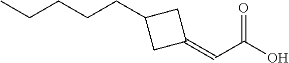 |
| XX | 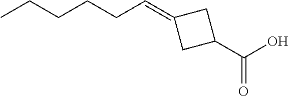 |
| XXI | 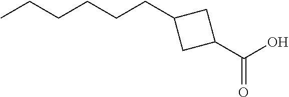 |
| XXII | 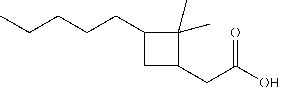 |
| XXIII | 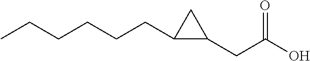 |
| XXIV | 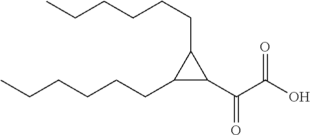 [SMILES: CCCCCCC1C(CCCCCC)C1C(=O)C(=O)O] |
| XXV | 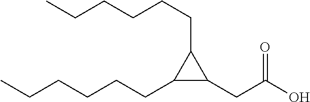 [SMILES: CCCCCCC1C(CCCCCC)C1CC(=O)O] |
| XXVI | 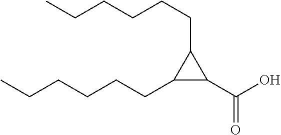 [SMILES: CCCCCCC1C(CCCCCC)C1C(=O)O] |
| XXVII | 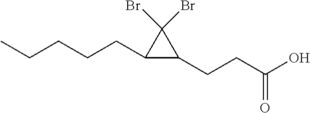 |
| XXVIII | 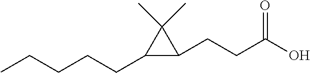 |
| XXIX | 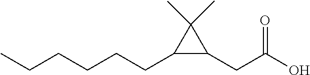 |
| XXX | 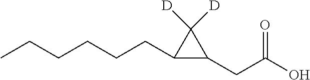 |
| XXXI | 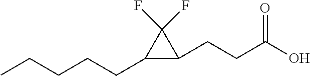 |
| XXXII | 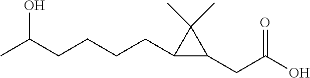 [SMILES: CC(O)CCCCC1C(CC(=O)O)C1(C)C] |
| XXXIII | 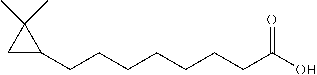 |
| XXXIV | 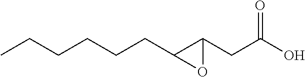 |
| XXXV | 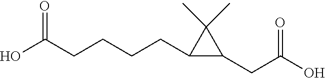 [SMILES: CC1(C)C(CCCCC(=O)O)C1CC(=O)O] |
| XXXVI | 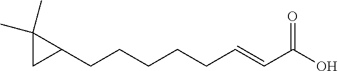 |
| XXXVII | 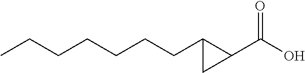 |
| XXXVIII | 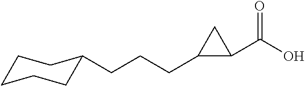 [SMILES: O=C(O)C1CC1CCCC1CCCCC1] |
| XXXIX | 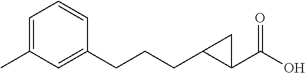 [SMILES: Cc1cccc(CCCC2CC2C(=O)O)c1] |
| XL | 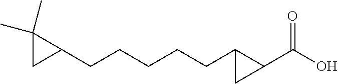 [SMILES: CC1(C)CC1CCCCCC1CC1C(=O)O] |
| XLI | 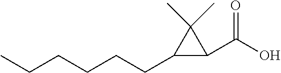 |
| XLII | 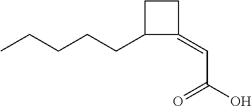 |
| XLIH | 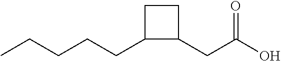 |
| XLIV | 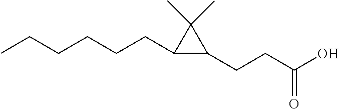 [SMILES: CCCCCCC1C(CCC(=O)O)C1(C)C] |
| XLV | 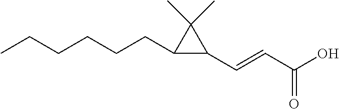 [SMILES: CCCCCCC1C(/C=C/C(=O)O)C1(C)C] |

30. The compound or salt thereof according to item 29, which is one of compounds I-IV, VII, IX, XIV, XVIII-XXI, XXVII, XXX, XXXI, XXXIII, XXXIV, XXXVII, XL, XLI, XLII or XLIII, or a salt thereof.
31. The compound or salt thereof according to any one of items 1 to 30, which is a metal salt of the compound.
32. The compound or salt thereof according to item 31, wherein the metal salt is a sodium salt.
33. The compound or salt thereof according to any one of items 1 to 32, which is a hydrochloride salt of the compound.
34. The compound or salt thereof according to any one of items 1 to 33, which is one of the salts depicted in Table 2:

TABLE 2

| Cmpd # | Structure |
|---|---|
| I | 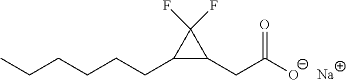 |
| II | 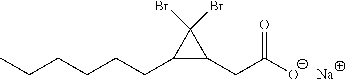 |
| III | 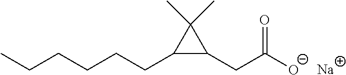 |
| IV | 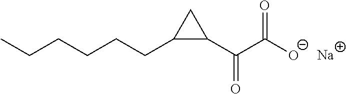 |
| V | 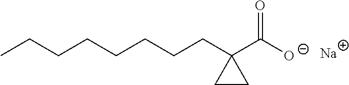 |
| VI | 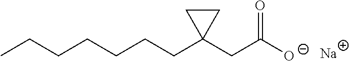 |
| VII | 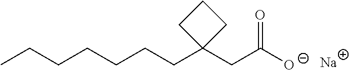 |
| VIII | 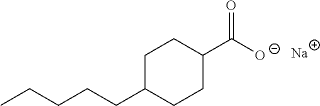 |
| IX | 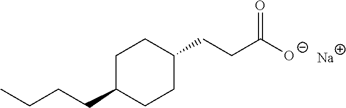 |
| X | 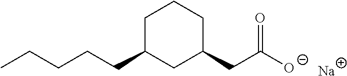 |
| XI | 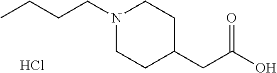 |
| XII | 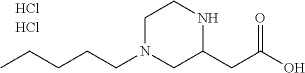 |
| XIII | 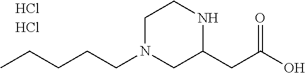 |
| XIV | 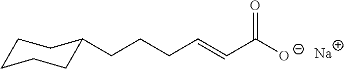 [SMILES: O=C([O-])/C=C/CCCC1CCCCC1 ; [Na+]] |
| XV | 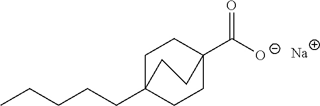 [SMILES: CCCCCC12CCC(C(=O)[O-])(CC1)CC2 ; [Na+]] |
| XVI | 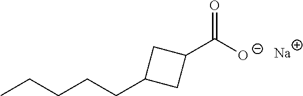 |
| XVII | 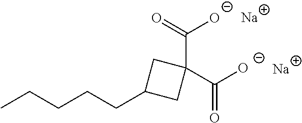 |
| XVIII | 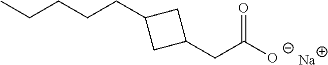 |
| XIX | 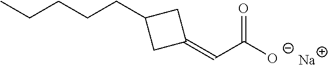 |
| XX | 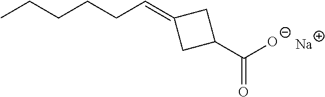 |
| XXI | 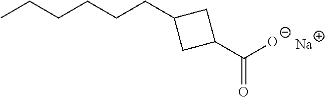 |
| XXII | 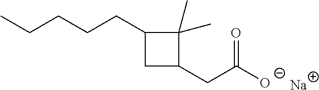 |
| XXIII | 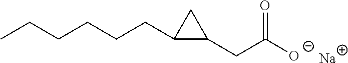 |
| XXIV | 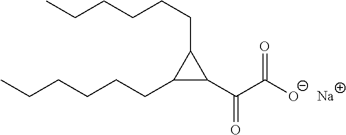 [SMILES: CCCCCCC1C(CCCCCC)C1C(=O)C(=O)[O-] ; [Na+]] |
| XXV | 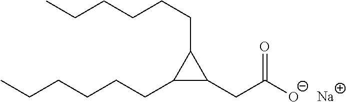 [SMILES: CCCCCCC1C(CCCCCC)C1CC(=O)[O-] ; [Na+]] |
| XXVI | 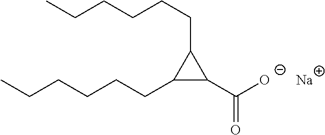 [SMILES: CCCCCCC1C(CCCCCC)C1C(=O)[O-] ; [Na+]] |
| XXVII | 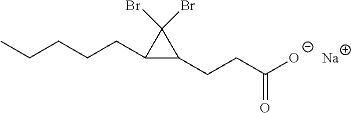 |
| XXVIII | 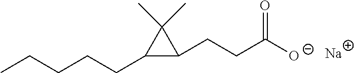 |
| XXIX | 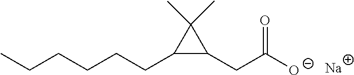 |
| XXX | 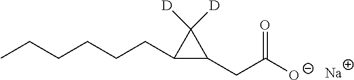 |
| XXXI | 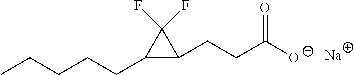 |
| XXXII | 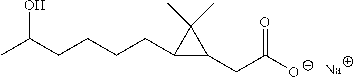 [SMILES: CC(O)CCCCC1C(CC(=O)[O-])C1(C)C ; [Na+]] |
| XXXIII | 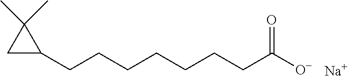 |
| XXXIV | 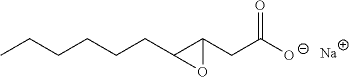 |
| XXXV | 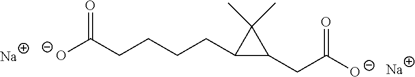 [SMILES: [Na+] ; CC1(C)C(CCCCC(=O)[O-])C1CC(=O)[O-] ; [Na+]] |
| XXXVI | 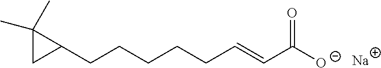 |
| XXXVII | 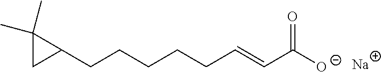 |
| XXXVIII | 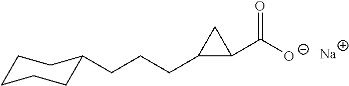 [SMILES: O=C([O-])C1CC1CCCC1CCCCC1 ; [Na+]] |
| XXXIX | 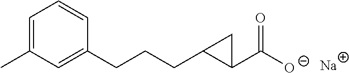 [SMILES: Cc1cccc(CCCC2CC2C(=O)[O-])c1 ; [Na+]] |
| XL | 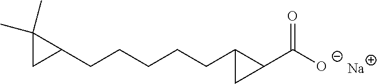 [SMILES: CC1(C)CC1CCCCCC1CC1C(=O)[O-] ; [Na+]] |
| XLI | 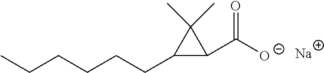 |
| XLII | 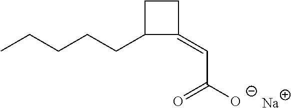 |
| XLIH | 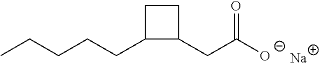 |
| XLIV | 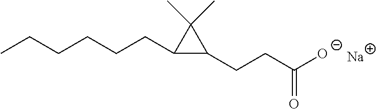 [SMILES: CCCCCCC1C(CCC(=O)O)C1(C)C ; [Na+]] |
| XLV | 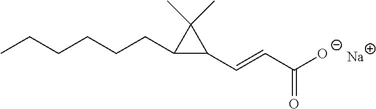 [SMILES: CCCCCCC1C(/C=C/C(=O)[O-])C1(C)C ; [Na+]] |

35. The compound or salt thereof according to item 34, which is one of salt I-IV, VII, IX, XIV, XVIII-XXI, XXVII, XXX, XXXI, XXXIII, XXXIV, XXXVII, XL, XLI, XLII or XLIII.
36. A composition comprising the compound or salt thereof according to any one of items 1 to 35 and a carrier or excipient.
37. A method for stimulating hematopoiesis or erythropoiesis in a subject in need thereof comprising administering to the subject an effective amount of the compound or salt thereof according to any one of items 1 to 35 or the composition of item 38.
38. A method for treating anemia or leukopenia in a subject in need thereof comprising administering to the subject an effective amount of the compound or salt thereof according to any one of items 1 to 35 or the composition of item 36.
39. The method of item 38, wherein the leukopenia and/or anemia is caused by chemotherapy.
40. The method of item 38, wherein the leukopenia and/or anemia is caused by bone marrow transplantation.
41. The method of any one of items 37 to 40, wherein the subject suffers from immunodeficiency.
42. A method for preventing and/or treating fibrosis in a subject in need thereof comprising administering to the subject an effective amount of the compound or salt thereof according to any one of items 1 to 35 or the composition of item 36.
43. The method of item 42, wherein the fibrosis is kidney fibrosis, lung fibrosis, liver fibrosis, heart fibrosis, bone marrow fibrosis or skin fibrosis.
44. A method for treating cancer in a subject in need thereof comprising administering to the subject an effective amount of the compound or salt thereof according to any one of items 1 to 35 or the composition of item 36.
45. A method for treating hypertension in a subject in need thereof comprising administering to the subject an effective amount of the compound or salt thereof according to any one of items 1 to 35 or the composition of item 36.
46. A method for treating a metabolic condition in a subject in need thereof comprising administering an effective amount of the compound or salt thereof according to any one of items 1 to 35 or the composition of item 36.
47. The method of item 46, wherein the metabolic condition is metabolic syndrome, pre-diabetes, or diabetes.
48. The method of item 46, wherein the diabetes is Type II diabetes.
49. The compound or salt thereof according to any one of items 1 to 35 or the composition of item 36, for use in stimulating hematopoiesis or erythropoiesis in a subject.
50. The compound or salt thereof according to any one of items 1 to 35 or the composition of item 36, for use in treating anemia or leukopenia in a subject.
51. The compound or salt thereof or composition for use according to item 50, wherein the leukopenia and/or anemia is caused by chemotherapy.
52. The compound or salt thereof or composition for use according to item 50, wherein the leukopenia and/or anemia is caused by bone marrow transplantation.
53. The compound or salt thereof or composition for use according to any one of items 49 to 52, wherein the subject suffers from immunodeficiency.
54. The compound or salt thereof according to any one of items 1 to 35 or the composition of item 36, for use in preventing and/or treating fibrosis in a subject.
55. The compound or salt thereof or composition for use according to item 54, wherein the fibrosis is kidney fibrosis, lung fibrosis, liver fibrosis, heart fibrosis, bone marrow fibrosis or skin fibrosis.
56. The compound or salt thereof according to any one of items 1 to 35 or the composition of item 36, for use in treating cancer in a subject.
57. The compound or salt thereof according to any one of items 1 to 35 or the composition of item 36, for use in treating hypertension in a subject.
58. The compound or salt thereof according to any one of items 1 to 35 or the composition of item 36, for use in treating a metabolic condition in a subject.
59. The compound or salt thereof or composition for use according to item 58, wherein the metabolic condition is metabolic syndrome, pre-diabetes, or diabetes.
60. The compound or salt thereof or composition for use according to item 59, wherein the diabetes is Type II diabetes.
61. Use of the compound or salt thereof according to any one of items 1 to 35 or the composition of item 36, for the manufacture of a medicament for stimulating hematopoiesis or erythropoiesis in a subject.
62. Use of the compound or salt thereof according to any one of items 1 to 35 or the composition of item 36, for stimulating hematopoiesis or erythropoiesis in a subject.
63. Use of the compound or salt thereof according to any one of items 1 to 35 or the composition of item 36, for the manufacture of a medicament for treating anemia or leukopenia in a subject.
64. Use of the compound or salt thereof according to any one of items 1 to 35 or the composition of item 36, for treating anemia or leukopenia in a subject.
65. The use according to item 63 or 64, wherein the leukopenia and/or anemia is caused by chemotherapy.
66. The use according to item 63 or 64, wherein the leukopenia and/or anemia is caused by bone marrow transplantation.
67. The use according to any one of items 61 to 66, wherein the subject suffers from immunodeficiency.
68. Use of the compound or salt thereof according to any one of items 1 to 35 or the composition of item 36, for the manufacture of a medicament for preventing and/or treating fibrosis in a subject.
69. Use of the compound or salt thereof according to any one of items 1 to 35 or the composition of item 36, for preventing and/or treating fibrosis in a subject.
70. The use of item 68 or 69, wherein the fibrosis is kidney fibrosis, lung fibrosis, liver fibrosis, heart fibrosis, bone marrow fibrosis or skin fibrosis.
71. Use of the compound or salt thereof according to any one of items 1 to 35 or the composition of item 36, for the manufacture of a medicament for treating hypertension in a subject.
72. Use of the compound or salt thereof according to any one of items 1 to 35 or the composition of item 36, for treating hypertension in a subject.
73. Use of the compound or salt thereof according to any one of items 1 to 35 or the composition of item 36, for the manufacture of a medicament for treating cancer in a subject.
74. Use of the compound or salt thereof according to any one of items 1 to 35 or the composition of item 36, for treating cancer in a subject.
75. Use of the compound or salt thereof according to any one of items 1 to 35 or the composition of item 36, for treating a metabolic condition in a subject.
76. Use of the compound or salt thereof according to any one of items 1 to 35 or the composition of item 36, for the manufacture of a medicament for treating a metabolic condition in a subject.
77. The use according to item 75 or 76, wherein the metabolic condition is metabolic syndrome, pre-diabetes, or diabetes.
78. The use according to item 77, wherein the diabetes is Type II diabetes.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 is a graph showing the effect of the sodium salt of 2-(3-hexyl-2,2-dimethylcyclopropyl)acetate (compound III) on white blood cell (WBC) count in cyclophosphamide-treated mice.

FIG. 2 is a graph showing the effect of compound III on spleen red blood cell (RBC) count in cyclophosphamide-treated mice.

FIG. 3 is a graph showing the effect of compound III on spleen white blood cell count in cyclophosphamide-treated mice.

FIG. 4 is a graph showing the effect of compound III and the sodium salt of 2-(2-hexylcyclopropyl)-2-oxoacetate (compound IV) on blood white blood cell count in cyclophosphamide-treated mice.

FIG. 5 is a graph showing the effect of compounds III and IV on bone marrow white blood cell count in cyclophosphamide treated mice.

FIG. 6 is a graph showing the effect of compounds I, III and IV on the concentration of serum albumin induced by doxorubicin in mice.

FIG. 7 is a graph showing the effect of compound XXX on the concentration of serum albumin induced by doxorubicin in mice.

FIG. 8 is a graph showing the effect of compounds IX and X on the concentration of serum albumin induced by doxorubicin in mice.

FIG. 9 is a graph showing the effect of compound III on body weight loss in an adenine-induced chronic kidney disease (CKD) mouse model.

FIGS. 10A-C are graphs showing the effect of compound III on red blood cell progenitors (FIG. 10A), hematocrit (FIG. 10B), and hemoglobin content (FIG. 10C) in an adenine-induced CKD mouse model.

FIGS. 11A-C are graphs showing the effect of compound III on glomerular filtration rate (GFR) (FIG. 11A), blood urea nitrogen (BUN) (FIG. 11B) and creatinine levels (FIG. 11C) in an adenine-induced CKD mouse model.

FIG. 12 is a graph showing the effect of compound III on survival in an adenine-induced CKD mouse model.

FIGS. 13A-D are graphs showing the effect of compound III on the expression of the pro-inflammatory genes MCP-1 (FIG. 13A), TNF-α (FIG. 13B), IL-6 (FIG. 13C) and IL-1β (FIG. 13D) in an adenine-induced CKD mouse model.

FIG. 14 is a graph showing the effect of compound III on the expression of the neutrophil gelatinase-associated lipocalin (NGAL) gene in an adenine-induced CKD mouse model.

FIGS. 15A-E are graphs showing the effect of compound III on the expression of the fibrosis marker genes Col1a1 (FIG. 15A), CTGF (FIG. 158), fibronectin (FIG. 15C) α-SMA (FIG. 15D) and MMP-2 (FIG. 15E) in an adenine-induced CKD mouse model.

FIGS. 16A and 16B are graphs showing the effect of compound III on serum creatinine (FIG. 16A) and urea (FIG. 168) levels in a 5/6 nephrectomized (Nx) rat model.

FIGS. 17A and 17B are graphs showing the effect of compound III on glomerular filtration rate (GFR) in a 5/6 nephrectomized (Nx) rat model. FIG. 17A shows the level of GFR over the entire study period, and FIG. 17B shows the changes in GFR vs. GFR at day 21.

FIG. 18 is a graph showing the effect of compound III on the percentage of animals having a serum creatinine level greater than 300 μmol/L, indicative of renal failure or end stage renal disease (ESRD) in a 5/6 nephrectomized (Nx) rat model.

FIG. 19 is a graph showing the effect of compound III on glomerulosclerosis, tubulointerstitial fibrosis, tubular dilatation, proteinaceous deposits, renal changes, mineralization, tubular basophilia and kidney inflammation in a 5/6 nephrectomized (Nx) rat model.

FIG. 20 is a graph showing the effect of compound III on serum triglyceride levels in a 5/6 nephrectomized (Nx) rat model.

FIG. 21 is a graph showing the effect of compound III or acetylsalicylic acid (ASA) on tumor growth in a syngeneic P815 tumor mice model.

FIG. 22 is a graph showing the effect of compound III on blood pressure in an animal model of diabetic/chronic kidney disease (DKD/CKD) induced by adenine supplementation and streptozotocin (STZAD).

### DETAILED DISCLOSURE

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure, such as Formula (I), with various substituents (R¹, R², etc.) and various radicals (alkyl, halogen atom, etc.) enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

Any and all combinations and subcombinations of the embodiments and features disclosed herein are encompassed by the present disclosure.

All methods described herein can be performed in any suitable order unless otherwise indicated herein or otherwise clearly contradicted by context.

The use of any and all examples, or exemplary language (e.g., “such as”) provided herein, is intended merely to better illustrate embodiments of the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Herein, the terms “alkyl”, “alkylene”, “alkenyl”, “alkenylene”, “alkynyl”, “alkynylene” and their derivatives (such as alkoxy, alkyleneoxy, etc.) have their ordinary meaning in the art.

For more certainty, herein:

| | |
|---|---|
| Term | Definition |

Saturated aliphatic hydrocarbons

| | |
|---|---|
| alkane | aliphatic hydrocarbon radical of general formula CₙH₂ₙ₊₂ |
| alkyl | monovalent alkane radical, general formula —CₙH₂ₙ₊₁ |
| alkylene | bivalent alkane radical, general formula —CₙH₂ₙ— |
| (also called alkanediyl) | |

Aliphatic hydrocarbons with double bond(s)

| | |
|---|---|
| alkene | aliphatic hydrocarbon radical, similar to an alkane but comprising |
| | at least one double bond |
| alkenyl | monovalent alkene radical, similar to an alkyl but comprising at |
| | least one double bond |
| alkenylene | bivalent alkene radical, similar to an alkylene but comprising at |
| | least one double bond |

Aliphatic hydrocarbons with triple bond(s)

| | |
|---|---|
| alkyne | aliphatic hydrocarbon radical, similar to an alkane but comprising |
| | at least one triple bond |
| alkynyl | monovalent alkyne radical, similar to an alkyl but comprising at |
| | least one triple bond |
| alkynylene | bivalent alkyne radical, similar to an alkylene but comprising at |
| | least one triple bond |

Aliphatic hydrocarbons with double and triple bond(s)

| | |
|---|---|
| alkenyne | aliphatic hydrocarbon radical, similar to an alkane but comprising |
| | at least one double bond and at least one triple bond |
| alkenynyl | monovalent alkenyne radical, similar to an alkyl but comprising at |
| | least one double bond and at least one triple bond |
| alkenynylene | bivalent alkenyne radical, similar to an alkylene but comprising at |
| | least one double bond and at least one triple bond |

It is to be noted that, unless otherwise specified, the hydrocarbon chains of the above groups can be linear or branched. Further, unless otherwise specified, these groups can in embodiments contain between 1 and 18 carbon atoms, in further embodiments between 1 and 12 carbon atoms, and in yet further embodiments between 1 and 6 carbon atoms or between 1 and 3 carbon atoms.

Herein, the term “cycloalkyl”, “aryl”, “heterocycloalkyl”, and “heteroaryl” have their ordinary meaning in the art. For more certainty, herein:

| Term | Definition |
|---|---|
| cycloalkane | monovalent saturated aliphatic hydrocarbon radical of general formula |
| | CₙH₂ₙ, wherein the carbon atoms are arranged in a ring (also called |
| | cycle). |
| cycloalkyl | monovalent cycloalkane radical |
| heterocycloalkane | cycloalkane wherein at least one of the carbon atoms is replaced by a |
| | heteroatom, such as nitrogen or oxygen. |
| heterocycloalkyl | monovalent heterocycloalkyl radical |
| arene | aromatic hydrocarbon presenting alternating double and single bonds |
| | between carbon atoms arranged in one or more rings. |
| aryl | monovalent arene radical |
| heteroarene | arene wherein at least one of the carbon atoms forming the ring(s) is |
| | replaced by a heteroatom, such as nitrogen or oxygen |
| heteroaryl | monovalent heteroarene radical |

Herein, a “heteroatom” is an atom other than a carbon atom or a hydrogen atom. In embodiments, the heteroatom is oxygen or nitrogen.

Herein, a “ring atom”, such as a ring carbon atom or a ring heteroatom, refers to an atom that forms (with other ring atoms) a ring of a cyclic compound, such as a cycloalkyl, an aryl, etc.

Herein, a “group substituted with one or more A, B, and/or C” means that one or more hydrogen atoms of the group may be replaced with groups selected from A, B, and C. Of note, the group do not need to be identical; one hydrogen atom may be replaced by A, while another may be replaced by B, etc.

In a first aspect, the present disclosure provides a compound of formula (I) or a salt thereof:

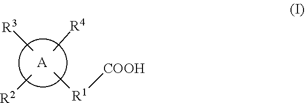

wherein:
A represents a 3- to 6-membered cycloalkane or heterocycloalkane, wherein the cycloalkane or heterocycloalkane are optionally bridged,
R¹ represents a covalent bond or an alkylene or alkenylene chain, wherein the alkylene or alkenylene chain is optionally substituted with ═O,
R² represents a hydrogen atom or an alkyl or alkenyl chain, wherein:
   the alkyl or alkenyl chain is optionally substituted with a hydroxy group, or
   the alkyl or alkenyl chain is optionally terminated with a carboxyl group or with a 3- to 6-membered cycloalkyl, heterocycloalkyl, aryl or heteroaryl, and
   the cycloalkyl, heterocycloalkyl, aryl, and heteroaryl are optimally substituted with one or more alkyl groups, and
R³ and R⁴ are identical to each other or different, are both attached to a same ring atom of A, and represent hydrogen atoms, deuterium atoms, halogen atoms, or methyl groups, or
R³ represents R², wherein R² is as defined above, and R⁴ represents a hydrogen atom,
R¹ and R² are attached on a same ring atom of A or on different ring atoms of A, wherein the atom of R¹, or of A if R¹ is a covalent bond, that bears the —COOH group is optionally substituted with a second —COOH group,
   wherein A, R¹ and R² are such that the shortest continuous chain of carbon atoms and, if present, heteroatoms linking:
the carbon atom or ring heteroatom in R² that is farthest from R¹ or, if R² represents a hydrogen atom, the ring carbon atom or ring heteroatom in A that is farthest from R¹
to the carbon atom of the COOH group terminating R¹
   is 9 to 11 atoms long,
   wherein the COOH group may be replaced by an isostere thereof.
   and
   wherein the compound is not

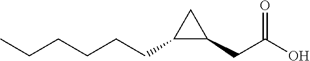

(cascarillic acid) or

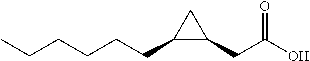

(cis-2-(2-hexylcyclopropyl)-acetic acid).

For more certainty, when counting the number of atoms in the “shortest continuous chain” in a compound in which R² is not a hydrogen atom, the carbon atom or ring heteroatom in R² that is farthest from R¹ is as follows:
When the alkyl or alkenyl chain in R² is not terminated with a 3- to 6-membered cycloalkyl, heterocycloalkyl, aryl or heteroaryl, the “carbon atom or ring heteroatom in R² that is farthest from R¹” is the terminal carbon atom of the alkyl or alkenyl chain.
   This also applies when the alkyl or alkenyl chain in R² is terminated with a carboxyl group. In this specific case, the terminal carbon atom of the alkyl or alkenyl chain is actually the carbon atom of the carboxyl (COOH) group.
   The hydroxy groups that can optionally be attached to R² are not counted as they are neither a carbon atom nor a ring heteroatom.
When the alkyl or alkenyl chain is terminated with an unsubstituted 3- to 6-membered cycloalkyl, heterocycloalkyl, aryl or heteroaryl, the “carbon atom or ring heteroatom in R² that is farthest from R¹” is the carbon atom or heteroatom of the cycloalkyl, heterocycloalkyl, aryl or heteroaryl that is the farthest from the point of attachment of the cycloalkyl, heterocycloalkyl, aryl or heteroaryl to the alkyl or alkenyl chain.
When the alkyl or alkenyl chain is terminated with a 3- to 6-membered cycloalkyl, heterocycloalkyl, aryl or heteroaryl and this cycloalkyl, heterocycloalkyl, aryl, or heteroaryl is substituted with one or more alkyl groups, the “carbon atom or ring heteroatom in R² that is farthest from R¹” is the terminal carbon atom of the alkyl group that substitutes the cycloalkyl, heterocycloalkyl, aryl, or heteroaryl.
   If several alkyl groups substitute the cycloalkyl, heterocycloalkyl, aryl, or heteroaryl, the “carbon atom or ring heteroatom in R² that is farthest from R¹” is the terminal carbon atom of the longest of these alkyl groups.

To illustrate how the atoms are counted in this “shortest continuous chain”, we provide below several compounds in which numeral “1” identifies the “carbon atom in R² that is farthest from R¹ or, if R² represents a hydrogen atom, the carbon atom or heteroatom in A that is farthest from R¹” and the highest numeral represent to the carbon atom of the COOH group terminating R¹.

Compound with atom count for the “shortest continuous
chain” and observations

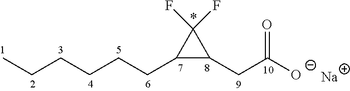

The carbon atom marked with a star (*) is not counted because
it is not part of the shortest continuous chain. Indeed,
counting this atom would have led to a chain with 11 atoms:

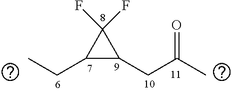

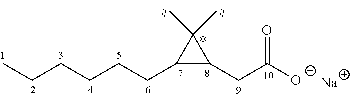

The carbon atom marked with a star (*) is not counted
because it is not part of the shortest continuous chain.
The methyl groups marked with a pound sign (#) are not
counted because they are R³ and R⁴, not R².

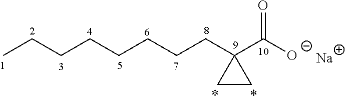

The carbon atoms marked with a star (*) are not counted
because they are not part of the shortest continuous chain.
This is an example of a compound in which R¹ represents
a covalent bond.

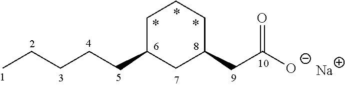

The carbon atoms marked with a star (*) are not counted
because they are not part of the shortest continuous chain.

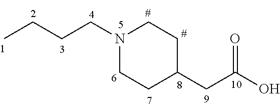

There are two “shortest continuous chains” of the same
length in this compound.
Indeed, the carbon atoms marked with a pound sign (#)
could have been counted instead of carbon atoms 6 and 7.
This is an example of a compound in which the shortest
continuous chain contains a heteroatom.

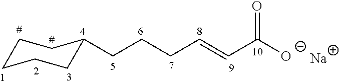 [SMILES: O=C([O-])/C=C/CCCC1CCCCC1 ; [Na+]]

There are two “shortest continuous chains” of the same
length in this compound.
Indeed, the carbon atoms marked with a pound sign (#)
could have been counted instead of carbon atoms 2 and 3.
This is an example of a compound in which R² is a hydrogen
atom. Therefore, the “shortest continuous chain”
starts with the carbon atom or heteroatom in A that is
farthest from R¹.

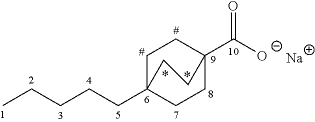 [SMILES: CCCCCC12CCC(C(=O)[O-])(CC1)CC2 ; [Na+]]

There are three “shortest continuous chains” of the
same length in this compound.
Indeed, the two carbon atoms marked with a pound sign (#)
or the two carbon atoms marked with a star (*)
could have been counted instead of carbon atoms 7 and 8.

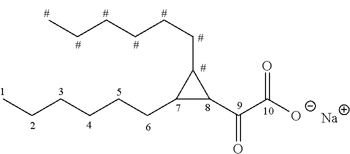 [SMILES: CCCCCCC1C(CCCCCC)C1C(=O)C(=O)[O-] ; [Na+]]

There are two “shortest continuous chains” of the
same length in this compound.
Indeed, the carbon atoms marked with a pound sign (#)
could have been counted instead of carbon atoms 1 to 7.
This is an example of a compound in which R³ represents
a R² group, which is identical to the other R² group.
If the R² groups had been different from one another,
the shortest of the two would have been used for counting
the “shortest continuous chains”.
This is also an example of a compound in which R¹ is
substituted (with C═O). Such substitutions do not affect the count.

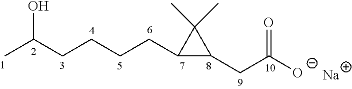 [SMILES: CC(O)CCCCC1C(CC(=O)[O-])C1(C)C ; [Na+]]

The oxygen (heteroatom) of the hydroxy group is not considered
since it is neither a carbon atom nor a ring heteroatom.

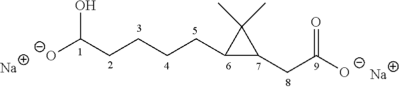 [SMILES: [Na+] ; CC1(C)C(CCCCC([O-])O)C1CC(=O)[O-] ; [Na+]]

This is an example of a compound in which R² is substituted
with a COOH group.

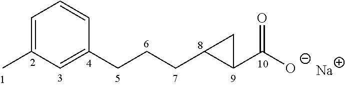 [SMILES: Cc1cccc(CCCC2CC2C(=O)[O-])c1 ; [Na+]]

This is an example of a compound in which R² is a chain
terminated by a substituted aryl group that is substituted
with an alkyl group. In this case, the carbon of the methyl
substituent on the aryl group was the carbon atom farthest from R¹.

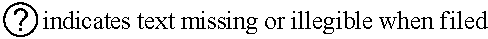

As noted above, A represents a 3- to 6-membered cycloalkane or heterocycloalkane. In embodiments, A represents a 3- to 6-membered cycloalkane. Preferred cycloalkanes include cyclopropane, cyclobutane, and cyclohexane. More preferred cycloalkanes include cyclopropane and cyclobutene. Preferred heterocycloalkanes include ethylene oxide

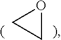

piperidine and piperazine. A more preferred heterocycloalkane is ethylene oxide.

As also noted above, the cycloalkane or heterocycloalkane in A can be bridged. Herein, a “bridged” cycloalkane or heterocycloalkane is a bridged bicyclic cycloalkane or heterocycloalkane in which two rings share three or more atoms, separating the two bridgehead atoms by a bridge containing at least one atom. For example, norbornane can be thought of as a pair of cyclopentane rings each sharing three of their five carbon atoms:

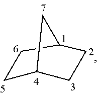

norbornane, also known as bicyclo[2.2.1]heptane

In an embodiment, the bridged cycloalkane or heterocycloalkane is bicyclo[2.2.2]octane.

In another embodiment, the cycloalkane or heterocycloalkane in A is unbridged.

As noted above, R¹ and R² can attached on a same ring atom or on different ring atoms of the cycloalkane or heterocycloalkane in A. In embodiments, R¹ and R² are attached on a same ring atom. In other embodiments, R¹ and R² are attached on different ring atoms of the cycloalkane or heterocycloalkane. In these embodiments, R¹ and R² are attached:
on ring atoms that are adjacent to each other,
on ring atoms that are separated by a single other ring atom, or
on ring atoms that are opposite each other.

In embodiments, R¹ and R² are attached on ring atoms that are adjacent to each other. In other embodiments, R¹ and R² are attached on ring atoms that are separated by a single other ring atom. In yet other embodiments, R¹ and R² are attached on ring atoms that are opposite each other.

In certain embodiments, A represents:
cyclopropane with R¹ and R² attached on a same atom of the cyclopropane,
cyclopropane with R¹ and R² attached on adjacent atoms of the cyclopropane,
ethylene oxide

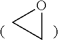

with R¹ and R² attached on adjacent ring atoms of the ethylene oxide
cyclobutane with R¹ and R² attached on a same ring atom of the cyclobutane,
cyclobutane with R¹ and R² attached on adjacent ring atoms of the cyclobutane,
cyclobutane with R¹ and R² attached on opposite ring atoms of the cyclobutane,
cyclohexane with R¹ and R² attached on opposite ring atoms of the cyclohexane,
cyclohexane with R¹ and R² attached on ring atoms of the cyclohexane that are separated by a single other ring atom,
piperidine with R¹ and R² attached on opposite ring atoms of the piperidine,
piperazine with R¹ and R² attached on ring atoms of the piperazine that are separated by a single other ring atom, or
bicyclo[2.2.2]octane with R¹ and R² attached on opposite ring atoms of the bicyclo[2.2.2]octane,

In further embodiment, A represents:
cyclopropane with R¹ and R² attached on adjacent atoms of the cyclopropane,
ethylene oxide

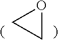

with R¹ and R² attached on adjacent ring atoms of the ethylene oxide
cyclobutane with R¹ and R² attached on a same ring atom of the cyclobutane,
cyclobutane with R¹ and R² attached on adjacent ring atoms of the cyclobutane,
cyclobutane with R¹ and R² attached on opposite ring atoms of the cyclobutane, or
cyclohexane with R¹ and R² attached on opposite ring atoms of the cyclohexane.

As noted above, R¹ represents a covalent bond or an alkylene or alkenylene chain. In embodiments, R¹ represents a covalent bond. In other embodiments, R¹ represents an alkylene chain. In yet other embodiments, R¹ represents an alkenylene chain. In preferred embodiments, R¹ represents a covalent bond or an alkylene chain. In embodiments, the alkylene or alkenylene chain in R¹ is a C₁-C₈ chain, a C₁-C₇ chain, a C₁-C₂ chain or a C₅-C₇ chain.

As noted above, the alkylene or alkenylene chain in R¹ is optionally substituted with ═O. In embodiments, the alkylene or alkenylene chain in R¹ is substituted with ═O. In another embodiment, the alkylene or alkenylene chain is unsubstituted.

As noted above, R² represents a hydrogen atom or an alkyl or alkenyl chain. In embodiments, R² represents a hydrogen atom. In embodiments, R² represents an alkyl chain. In embodiments, R² represents an alkenyl chain. In preferred embodiments, R² represents an alkyl or alkenyl chain, more preferably and alkyl chain. In embodiments, the alkyl or alkenyl chain in R² is a C₁-C₈ chain, preferably a C₂-C₈ chain, more preferably a C₄-C₈ chain, yet more preferably a C₄-C₇ chain, most preferably a C₅-C₇ chain.

As noted above, the alkyl or alkenyl chain in R² is optionally substituted with a hydroxy group. In embodiments, the alkyl or alkenyl chain in R² is substituted with a hydroxy group. In preferred embodiments, the alkyl or alkenyl chain in R² is unsubstituted.

As noted above, the alkyl or alkenyl chain in R² is optionally terminated with a carboxyl group or with a 3- to 6-membered cycloalkyl, heterocycloalkyl, aryl or heteroaryl. In embodiments, the alkyl or alkenyl chain in R² is optionally terminated with a 3- to 6-membered cycloalkyl, heterocycloalkyl, aryl or heteroaryl. In more preferred embodiments, the alkyl or alkenyl chain in R² is terminated with a carboxyl group. In yet more preferred embodiments, the alkyl or alkenyl chain in R² is terminated with hydrogen atoms only. Preferred 3- to 6-membered cycloalkyl, heterocycloalkyl, aryl or heteroaryl terminating the alkyl or alkenyl chain in R² include cyclopropyl, cyclobutyl, cyclohexyl, and phenyl.

As noted above, the cycloalkyl, heterocycloalkyl, aryl or heteroaryl terminating the alkyl or alkenyl in R² are optimally substituted with one or more alkyl groups. In embodiments, these cycles are substituted with one or more alkyl groups, preferably one or two alkyl groups, preferably two alkyl groups. These alkyl groups can be identical to one another or different, preferably they are identical. These alkyl groups can be on a same or on different ring atoms of these cycles, preferably on a same ring atom, especially when there are two alkyl groups. In preferred embodiments, the cycloalkyl, heterocycloalkyl, aryl or heteroaryl terminating the alkyl or alkenyl in R² is:
cyclopropyl substituted with two identical or different, preferably identical, alkyl groups on the same ring atom,
cyclohexyl unsubstituted,
phenyl substituted with one alkyl group.

In other embodiments, the cycloalkyl, heterocycloalkyl, aryl or heteroaryl terminating the alkyl or alkenyl in R² are unsubstituted.

As noted above, either:
R³ and R⁴ are identical to each other or different and represent hydrogen atoms, deuterium atoms, halogen atoms, or methyl groups, or
R³ represents R², wherein R² is as defined above, and R⁴ represents hydrogen.

Thus, in embodiments, R³ and R⁴ are identical to each other, are both attached to a same ring atom of A, and represent hydrogen atoms, deuterium atoms, halogen atoms, or methyl groups. Preferred halogen atoms include F and Br. Generally speaking, R³ and R⁴ preferably represent hydrogen atoms, halogen atoms, or methyl groups; and more preferably hydrogen atoms. In embodiments where A represent cyclopropane, R³ and R⁴ may preferably represent halogen atoms or methyl groups.

In other embodiments, R³ represents R², wherein R² is as defined above including preferred embodiments thereof, and R⁴ represents a hydrogen atom.

As noted above, the atom of R¹ that bears the —COOH group is optionally substituted with a second —COOH group. When R¹ is a covalent bond, it is the atom of A that bears the (first) —COOH group the atom of A that can be optionally substituted with a second —COOH group.

The term “isostere” (or “(bio)isostere”) refer to a group groups that exhibit similar volume, shape, and/or physicochemical properties and that can produce broadly similar biological effects as another group. The (bio)isostere of the carboxylic acid (COOH) group may be a hydroxamic acid group, a phosphonic or phosphinic acid group, a sulphonic acid group, a sulfonamide group, an acylsulfonamic group or a sulfonylurea group (see Ballatore et al., Carboxylic Acid (Bio)Isosteres in Drug Design, ChemMedChem. 2013, 8(3): 385-395).

In an embodiment, the compound or salt thereof is one of the compounds depicted in Table 1, or a salt thereof:

TABLE 1

| Cmpd # | Structure |
|---|---|
| I | 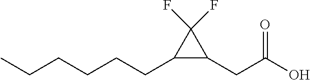 |
| II | 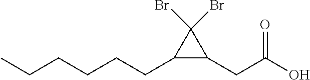 |
| III | 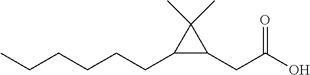 |
| IV | 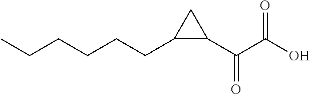 |
| V | 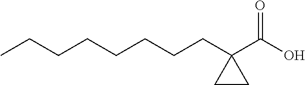 |
| VI | 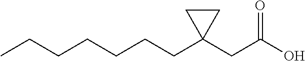 |
| VII | 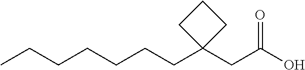 |
| VIII | 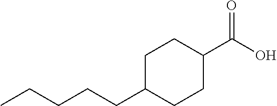 |
| IX | 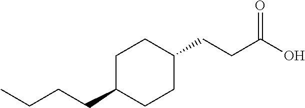 |
| X | 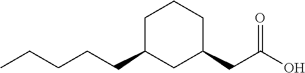 |
| XI | 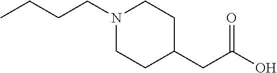 |
| XII | 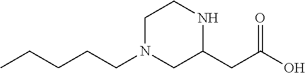 |
| XIII | 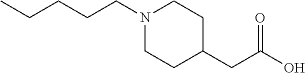 |
| XIV | 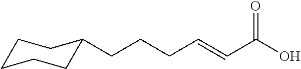 [SMILES: O=C(O)/C=C/CCCC1CCCCC1] |
| XV | 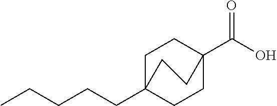 [SMILES: CCCCCC12CCC(C(=O)O)(CC1)CC2] |
| XVI | 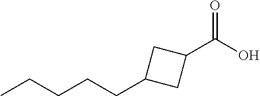 |
| XVII | 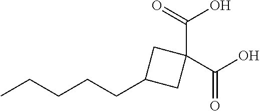 |
| XVIII | 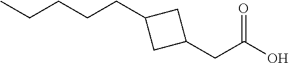 |
| XIX | 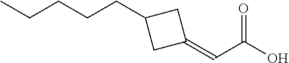 |
| XX | 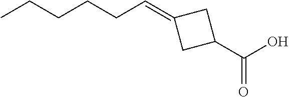 |
| XXI | 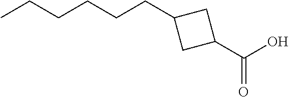 |
| XXII | 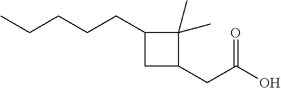 |
| XXIII | 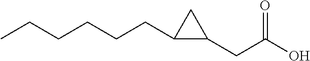 |
| XXIV | 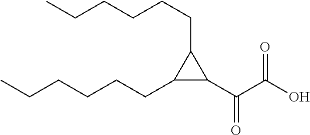 [SMILES: CCCCCCC1C(CCCCCC)C1C(=O)C(=O)O] |
| XXV | 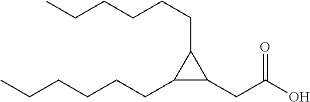 [SMILES: CCCCCCC1C(CCCCCC)C1CC(=O)O] |
| XXVI | 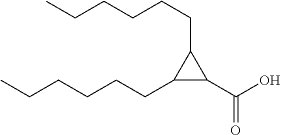 [SMILES: CCCCCCC1C(CCCCCC)C1C(=O)O] |
| XXVII | 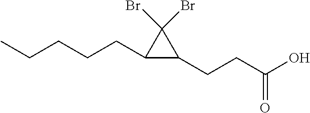 |
| XXVIII | 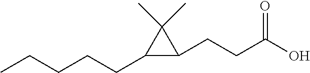 |
| XXIX | 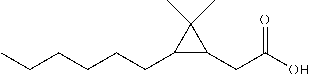 |
| XXX | 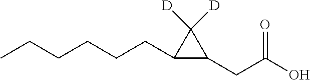 |
| XXXI | 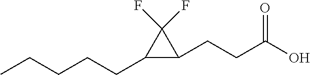 |
| XXXII | 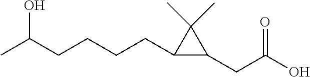 [SMILES: CC(O)CCCCC1C(CC(=O)O)C1(C)C] |
| XXXIII | 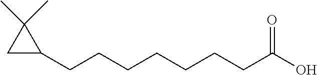 |
| XXXIV | 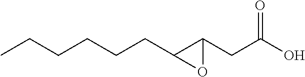 |
| XXXV | 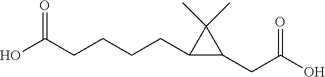 [SMILES: CC1(C)C(CCCCC(=O)O)C1CC(=O)O] |
| XXXVI | 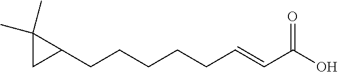 |
| XXXVII | 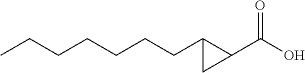 |
| XXXVIII | 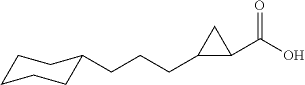 [SMILES: O=C(O)C1CC1CCCC1CCCCC1] |
| XXXIX | 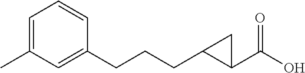 [SMILES: Cc1cccc(CCCC2CC2C(=O)O)c1] |
| XL | 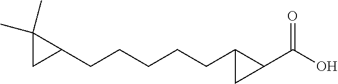 [SMILES: CC1(C)CC1CCCCCC1CC1C(=O)O] |
| XLI | 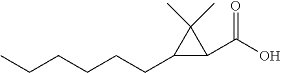 |
| XLII | 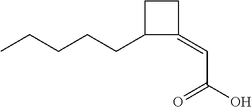 |
| XLIH | 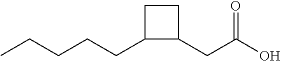 |
| XLIV | 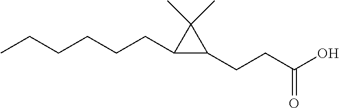 [SMILES: CCCCCCC1C(CCC(=O)O)C1(C)C] |
| XLV | 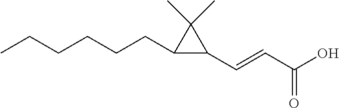 [SMILES: CCCCCCC1C(/C=C/C(=O)O)C1(C)C] |

In an embodiment, the compound or salt thereof is one of compounds I-IV, VII, IX, XIV, XVIII-XXI, XXVII, XXX, XXXI, XXXIII, XXXIV, XXXVII, XL, XLI, XLII or XLIII, or a salt thereof.

Salts

In an embodiment, a salt of a compound disclosed herein is a pharmaceutically acceptable salt. The term “pharmaceutically acceptable salt” refers to salts of compounds disclosed herein that are pharmacologically acceptable and substantially non-toxic to the subject to which they are administered. More specifically, these salts retain the biological effectiveness and properties of the compounds disclosed herein and are formed from suitable non-toxic organic or inorganic acids or bases.

For example, these salts include acid addition salts of the compounds disclosed herein which are sufficiently basic to form such salts. Such acid addition salts include acetates, adipates, alginates, lower alkanesulfonates such as a methanesulfonates, trifluoromethanesulfonates or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like. In an embodiment, the pharmaceutically acceptable acid salt of a compound disclosed herein is a hydrochloride salt, including a dihydrochloride salt.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Also, where the compounds disclosed herein are sufficiently acidic, the salts include base salts formed with an inorganic or organic base. Such salts include alkali metal salts such as sodium, lithium, and potassium salts; alkaline earth metal salts such as calcium and magnesium salts; metal salts such as aluminium salts, iron salts, zinc salts, copper salts, nickel salts and a cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts. In an embodiment, the pharmaceutically acceptable base salt of a compound disclosed herein is a metal salt, preferably a sodium salt, including a disodium salt.

Such salts can be formed by those skilled in the art using standard techniques (See, e.g., H. Ansel et al., Pharmaceutical Dosage Forms and Drug Delivery Systems (6^{th} Ed. 1995) at pp. 196 and 1456-1457). Salts of the compounds disclosed herein may be formed, for example, by reacting the compound with an amount of acid or base, such as an equivalent amount, in a medium such as one in which the salt precipitates or in an aqueous medium followed by lyophilization.

In an embodiment, the salt of the compound is one of the salts depicted in Table 2:

TABLE 2

| Salt of Cmpd # | Structure |
|---|---|
| I | 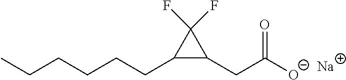 |
| II | 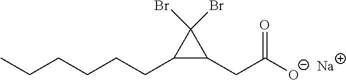 |
| III | 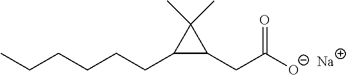 |
| IV | 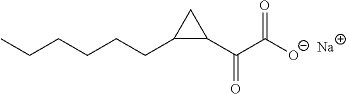 |
| V | 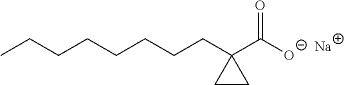 |
| VI | 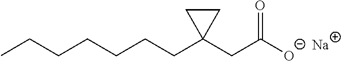 |
| VII | 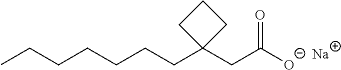 |
| VIII | 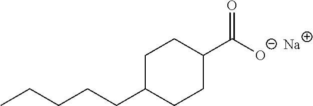 |
| IX | 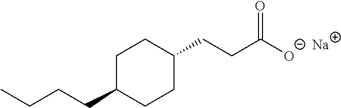 |
| X | 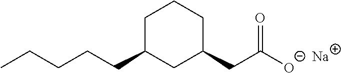 |
| XI | 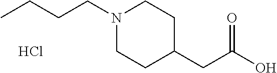 |
| XII | 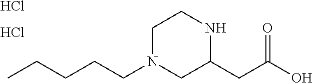 |
| XIII | 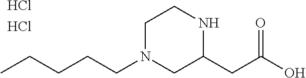 |
| XIV | 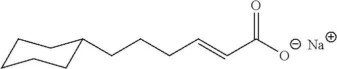 [SMILES: O=C([O-])/C=C/CCCC1CCCCC1 ; [Na+]] |
| XV | 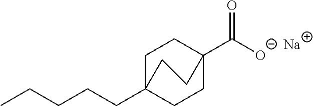 [SMILES: CCCCCC12CCC(C(=O)[O-])(CC1)CC2 ; [Na+]] |
| XVI | 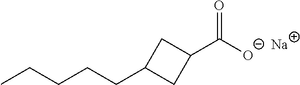 |
| XVII | 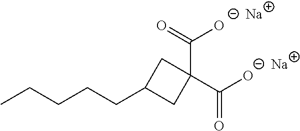 |
| XVIII | 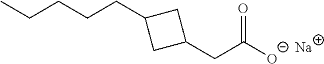 |
| XIX | 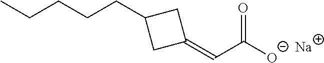 |
| XX | 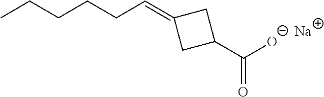 |
| XXI | 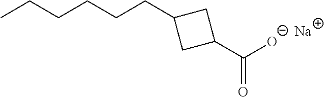 |
| XXII | 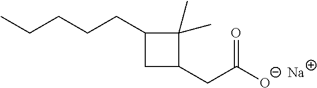 |
| XXIII | 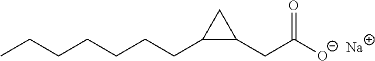 |
| XXIV | 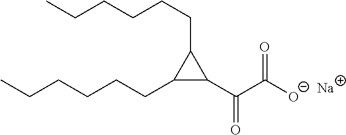 [SMILES: CCCCCCC1C(CCCCCC)C1C(=O)C(=O)[O-] ; [Na+]] |
| XXV | 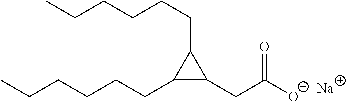 [SMILES: CCCCCCC1C(CCCCCC)C1CC(=O)[O-] ; [Na+]] |
| XXVI | 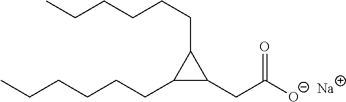 [SMILES: CCCCCCC1C(CCCCCC)C1CC(=O)[O-] ; [Na+]] |
| XXVII | 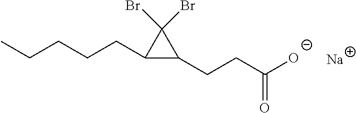 |
| XXVIII | 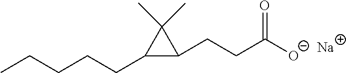 |
| XXIX | 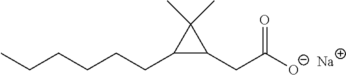 |
| XXX | 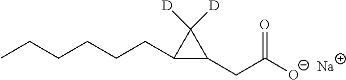 |
| XXXI | 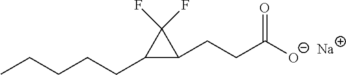 |
| XXXII | 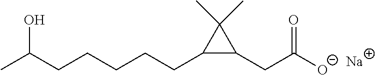 [SMILES: CC(O)CCCCCC1C(CC(=O)[O-])C1(C)C ; [Na+]] |
| XXXIII | 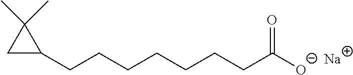 |
| XXXIV | 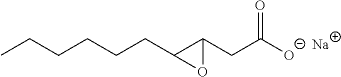 |
| XXXV | 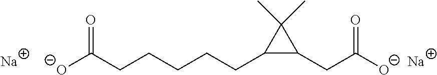 [SMILES: [Na+] ; CC1(C)C(CCCCCC(=O)[O-])C1CC(=O)[O-] ; [Na+]] |
| XXXVI | 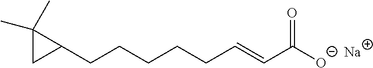 |
| XXXVII | 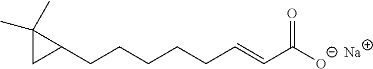 |
| XXXVIII | 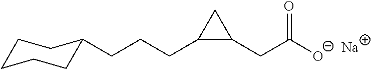 [SMILES: O=C([O-])CC1CC1CCCC1CCCCC1 ; [Na+]] |
| XXXIX | 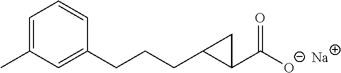 [SMILES: Cc1cccc(CCCC2CC2C(=O)[O-])c1 ; [Na+]] |
| XL | 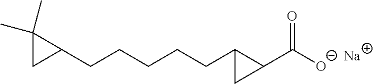 [SMILES: CC1(C)CC1CCCCCC1CC1C(=O)[O-] ; [Na+]] |
| XLI | 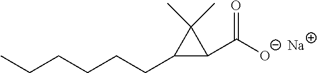 |
| XLII | 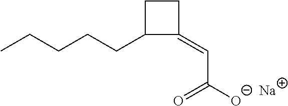 |
| XLIII | 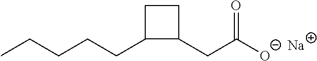 |
| XLIV | 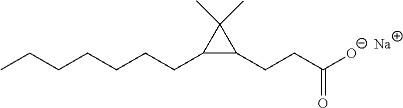 [SMILES: CCCCCCCC1C(CCC(=O)[O-])C1(C)C ; [Na+]] |
| XLV | 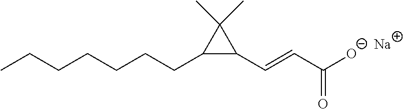 [SMILES: CCCCCCCC1C(/C=C/C(=O)[O-])C1(C)C ; [Na+]] |

Enantiomers, Isomers and Tautomers

The compounds described herein, or their pharmaceutically acceptable salts, may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)-. The present disclosure is intended to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively, specific enantiomers may be synthesized by asymmetric synthesis using optically active reagents, substrates, catalysts, or solvents or by converting one enantiomer to another by asymmetric transformation.

In addition, the present disclosure embraces all geometric and positional isomers. For example, if a compound disclosed herein incorporates a double bond or a fused ring, both the cis- and trans-forms, as well as mixtures, are embraced within the scope of the disclosure.

Within the present disclosure it is to be understood that a compound disclosed herein may exhibit the phenomenon of tautomerism and that the formulae drawn within this specification can represent only one of the possible tautomeric forms. It is to be understood that the disclosure encompasses any tautomeric form and is not to be limited merely to any one tautomeric form utilized within the formulae drawn.

It is also to be understood that certain compounds may exhibit polymorphism, and that the disclosure encompasses all such forms.

Certain compounds disclosed herein may exist in Zwitterionic form and the present invention includes Zwitterionic forms of these compounds and mixtures thereof.

Prodrugs, Esters

In certain embodiments, the compounds disclosed herein are present in the form of a prodrug. Examples of the latter include the pharmaceutically acceptable esters or amides obtained upon reaction of alcohols or amines, including amino acids, with free acids, such as the free acids defined by Formula I. The term “ester(s)”, as employed herein, refers to compounds disclosed herein or salts thereof in which hydroxy groups have been converted to the corresponding esters using, for example, inorganic or organic anhydrides, acids or acid chlorides. Esters for use in pharmaceutical compositions will be pharmaceutically acceptable esters, but other esters may be useful in the production of the compounds disclosed herein. The term “pharmaceutically acceptable ester” refers to esters of compounds disclosed herein that are pharmacologically acceptable and substantially non-toxic to the subject to which they are administered. More specifically, these esters retain the biological effectiveness and properties of the compounds and act as prodrugs which, when absorbed into the bloodstream of a warm-blooded animal, cleave in such a manner as to produce the parent alcohol. Further information concerning examples of and the use of esters for the delivery of pharmaceutical compounds is available in Design of Prodrugs. Bundgaard H ed. (Elsevier, 1985). See also, H. Ansel et al., Pharmaceutical Dosage Forms and Drug Delivery Systems (6th Ed. 1995) at pp. 108-109; Krogsgaard-Larsen, et al., Textbook of Drug Design and Development (2d Ed. 1996) at pp. 152-191.

Solvates

One or more compounds disclosed herein may exist in unsolvated as well as solvated forms with solvents such as water, ethanol, and the like, and it is intended that the disclosure embrace both solvated and unsolvated forms.

“Solvate” means a physical association of a compound disclosed herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Solvates for use in pharmaceutical compositions will be pharmaceutically acceptable esters, but other solvates may be useful in the production of the compounds disclosed herein.

As used herein, the term “pharmaceutically acceptable solvates” means solvates of compounds disclosed herein that are pharmacologically acceptable and substantially non-toxic to the subject to which they are administered. More specifically, these solvates retain the biological effectiveness and properties of the compounds disclosed herein and are formed from suitable non-toxic solvents.

Non-limiting examples of suitable solvates include ethanolates, methanolates, and the like, as well as hydrates, which are solvates wherein the solvent molecules are H₂O.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the compound disclosed herein in desired amounts of the desired solvent (organic or water or mixtures thereof) at a higher than ambient temperature, and cooling the solution at a rate sufficient to form crystals which are then isolated by standard methods. Analytical techniques such as, for example infrared spectroscopy, show the presence of the solvent (or water) in the crystals as a solvate (or hydrate).

Pharmaceutical Compositions

In another aspect, the present disclosure provides a composition comprising a compound formula (I) or salt thereof disclosed herein and a carrier or excipient, in a further embodiment a pharmaceutically acceptable carrier or excipient. Such compositions may be prepared in a manner well known in the pharmaceutical art. Supplementary active compounds can also be incorporated into the composition. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. Therapeutic formulations are prepared using standard methods known in the art by mixing the active ingredient having the desired degree of purity with one or more optional pharmaceutically acceptable carriers, excipients and/or stabilizers (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). In an embodiment, the pharmaceutical composition is an oral formulation or dosage form, for example a pill, capsule or tablet.

An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, the pharmaceutical composition includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL®), sodium croscarmellose, sodium starch glycolate (EXPLOTAB®, PRIMO JEL®) crosslinked polyvinylpolypyrrolidone (PLASONE-XL®), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, caranuba wax, xanthan gum, starch, stearate(s), stearic acid, stearic monoglyceride and stearyl alcohol.

Examples of thickening agents include talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol and polyethylene glycol), surfactants and terpenes.

Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compound or salt thereof. Other potentially useful parenteral delivery systems for compounds/compositions of the disclosure include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

Methods and Uses of the Compounds and Compositions

In another aspect, the present disclosure relates to a method for stimulating hematopoiesis or erythropoiesis in a subject in need thereof comprising administering to the subject an effective amount of a compound of formula (I), salt thereof or composition disclosed herein. The present disclosure also relates to the use of a compound of formula (I), salt thereof or composition disclosed herein for stimulating hematopoiesis or erythropoiesis in a subject, or for the manufacture of a medicament for stimulating hematopoiesis or erythropoiesis in a subject. The present disclosure also relates to a compound of formula (I), salt thereof or composition disclosed herein for use in stimulating hematopoiesis or erythropoiesis in a subject.

In another aspect, the present disclosure relates to a method for treating anemia or leukopenia in a subject in need thereof comprising administering to the subject an effective amount of a compound of formula (I), salt thereof or composition disclosed herein. The present disclosure also relates to the use of a compound of formula (I), salt thereof or composition disclosed herein for treating anemia in a subject, or for the manufacture of a medicament for treating anemia in a subject. The present disclosure also relates to a compound of formula (I), salt thereof or composition disclosed herein for use in treating anemia in a subject.

Leukopenia and anemia may be caused, for example, by chemotherapy (e.g., chemotherapy-induced anemia), radiotherapy and cancers (e.g., cancer-related anemia). Thus, in an embodiment, the subject suffers from anemia and/or leukopenia caused by chemotherapy or radiotherapy. A compound of formula (I), salt thereof or composition disclosed herein may be administered/used before, during and/or after chemotherapy or radiotherapy.

The compound of formula (I), salt thereof or composition disclosed herein may be also be used after bone marrow transplantation in order to stimulate bone marrow stem cells and immune reconstitution.

The compound of formula (I), salt thereof or composition disclosed herein may be administered/used in a subject suffering from immunodeficiency, e.g., B-cell deficiency, T-cell deficiency, or neutropenia. In an embodiment, the immunodeficiency is a secondary immunodeficiency (acquired immunodeficiency) which may be caused by several factors, e.g., immunosuppressive agents, malnutrition, aging, particular medications (e.g., chemotherapy, disease-modifying antirheumatic drugs, immunosuppressive drugs after organ transplants, glucocorticoids), environmental toxins like mercury and other heavy metals, pesticides and petrochemicals like styrene, dichlorobenzene, xylene, and ethylphenol, diseases such as cancer (particularly those of the bone marrow and blood cells (leukemia, lymphoma, multiple myeloma), and certain chronic infections such as HIV infection.

In another aspect, the present disclosure relates to a method for preventing and/or treating fibrosis, e.g., organ fibrosis, in a subject in need thereof comprising administering to the subject an effective amount of a compound of formula (I), salt thereof or composition disclosed herein. The present disclosure also relates to the use of a compound of formula (I), salt thereof or composition disclosed herein for preventing and/or treating fibrosis, e.g., organ fibrosis, in a subject, or for the manufacture of a medicament for preventing and/or treating fibrosis, e.g., organ fibrosis, in a subject. The present disclosure also relates to a compound of formula (I), salt thereof or composition disclosed herein for use in preventing and/or treating fibrosis, e.g., organ fibrosis, in a subject.

In an embodiment, the organ fibrosis is kidney fibrosis, lung fibrosis, liver fibrosis, heart fibrosis, bone marrow fibrosis or skin fibrosis. In an embodiment, the organ fibrosis is kidney fibrosis. In another embodiment, the organ fibrosis is lung fibrosis. In another embodiment, the organ fibrosis is liver fibrosis. In another embodiment, the organ fibrosis is heart fibrosis. In another embodiment, the organ fibrosis is skin fibrosis. In another embodiment, the organ fibrosis is bone marrow fibrosis.

In an embodiment, the fibrosis occurs in two or more organs. In an embodiment, the fibrosis is associated with a disease, for example an inherited disease or a chronic disease. In a further embodiment, the fibrosis is associated with Alström Syndrome, which is an autosomal recessive, single gene disorder caused by mutations in ALMS1. Alström Syndrome is multisystemic, with cone-rod retinal dystrophy leading to juvenile blindness, sensorineural hearing loss, obesity, insulin resistance with hyperinsulinemia, and type 2 diabetes mellitus. Very high incidences of additional disease phenotypes that may severely affect prognosis and survival include endocrine abnormalities, dilated cardiomyopathy, pulmonary fibrosis and restrictive lung disease, and progressive hepatic and renal failure. Fibrotic infiltrations of multiple organs including kidney, heart, liver, lung, urinary bladder, gonads, and pancreas, are also commonly observed in patients with Alström Syndrome. Thus, in an embodiment, the present disclosure relates to a method for treating Alström Syndrome (e.g., for reducing the severity and/or progression of Alström Syndrome) in a subject in need thereof comprising administering to the subject an effective amount of a compound, salt thereof or composition disclosed herein.

The term “lung fibrosis” or “pulmonary fibrosis” refers to the formation or development of excess fibrous connective tissue (fibrosis) in the lung thereby resulting in the development of scarred (fibrotic) tissue. More precisely, pulmonary fibrosis is a chronic disease that causes swelling and scarring of the alveoli and interstitial tissues of the lungs. The scar tissue replaces healthy tissue and causes inflammation. This chronic inflammation is, in turn, the prelude to fibrosis. This damage to the lung tissue causes stiffness of the lungs which subsequently makes breathing more and more difficult.

Pulmonary fibrosis may arise from many different causes which include microscopic damage to the lungs induced by inhalation of small particles (asbestos, ground stone, metal dust, particles present in cigarette smoke, silica dust, etc.). Alternatively, pulmonary fibrosis may arise as a secondary effect of other diseases (autoimmune disease, viral or bacterial infections, chronic obstructive pulmonary disease (COPD), etc.). Certain drugs such as cytotoxic agents (e.g. bleomycin, busulfan and methotrexate); antibiotics (e.g. nitrofurantoin, sulfasalazine); antiarrhythmics (e.g. amiodarone, tocainide); anti-inflammatory medications (e.g. gold, penicillamine); illicit drugs (e.g. crack, cocaine, heroin) also can cause pulmonary fibrosis. However, when pulmonary fibrosis appears without a known cause, it is referred to as “idiopathic” or idiopathic pulmonary fibrosis (IPF). In an embodiment, the lung fibrosis is idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension.

The term “liver fibrosis” or “hepatic fibrosis” means the formation or development of excess fibrous connective tissue (fibrosis) in the liver thereby resulting in the development of scarred (fibrotic) tissue. The scarred tissue replaces healthy tissue by the process of fibrosis and leads to subsequent cirrhosis of the liver. Liver fibrosis results from chronic damage to the liver in conjunction with the accumulation of ECM proteins, which is a characteristic of most types of chronic liver diseases. The main causes of liver fibrosis in industrialized countries include HBV infection, chronic HCV infection, schistosomiasis, auto-immune hepatitis, primary biliary cirrhosis, drug reaction, exposure to toxins, alcohol abuse, and nonalcoholic fatty liver disease/nonalcoholic steatohepatitis (NAFLD/NASH). The accumulation of ECM proteins distorts the hepatic architecture by forming a fibrous scar, and the subsequent development of nodules of regenerating hepatocytes defines cirrhosis. Cirrhosis produces hepatocellular dysfunction and increased intrahepatic resistance to blood flow, which result in hepatic insufficiency and portal hypertension, respectively. In an embodiment, the subject suffers from a chronic liver disease, such as NAFLD/NASH.

The term “skin fibrosis” or “dermal fibrosis” means the excessive proliferation of epithelial cells or fibrous connective tissue (fibrosis) thereby resulting in the development of scarred (fibrotic) tissue. The term “skin fibrosis” as used herein encompasses the fibrosis of any skin tissue and epithelial cells including, without limitation, blood vessels and veins, internal cavity of an organ or a gland such as ducts of submandibular, gallbladder, thyroid follicles, sweat gland ducts, ovaries, kidney; epithelial cells of gingival, tongue, palate, nose, larynx, oesophagus, stomach, intestine, rectum, anus and vagina; derma, scar, skin and scalp. Skin fibrosis occurs in several diseases or conditions including scleroderma, nephrogenic fibrosing dermopathy, mixed connective tissue disease, scleromyxedema, scleroderma, eosinophilic fasciitis, cutaneous Graft-versus-Host-Disease (GvHD), excessive scarring after trauma (injury, burn, surgery), hypertrophic scars, keloids, lipodermatosclerosis, collagenomas, carcinogenesis, ulcers (diabetic foot ulcer, a venous leg ulcer or a pressure ulcer) as well as exposures to chemicals, physical agents or radiations. Despite this variety of causes and disease-specific pathophysiologic processes leading to skin fibrosis, the cellular and molecular mechanisms of excessive extracellular matrix accumulation in the skin are fairly universal.

In an embodiment, a compound or composition disclosed herein improves wound healing, i.e. reduces scarring following skin injury.

The term “cardiac fibrosis” or “heart fibrosis” means an abnormal thickening of the heart valves due to inappropriate proliferation of cardiac fibroblasts but more commonly refers to the proliferation of fibroblasts in the cardiac muscle. Fibrocyte cells normally secrete collagen, and function to provide structural support for the heart. When over-activated this process causes thickening and fibrosis of the valve, with white tissue building up primarily on the tricuspid valve, but also occurring on the pulmonary valve. The thickening and loss of flexibility eventually may lead to valvular dysfunction and right-sided heart failure. Cardiac fibrosis occurs in several diseases or conditions including myocardial infarction, gastrointestinal carcinoid tumors of the mid-gut (which sometimes release large amounts of serotonin into the blood that promotes cardiac fibrosis), uses of agonists of the 5-HT_{2B} receptors (e.g., weight loss drugs such as fenfluramine and chlorphentermine, and antiparkinson drugs such as pergolide and cabergoline), use of appetite suppressant drugs such as fenfluramine, chlorphentermine and aminorex, uses of antimigraine drugs such as ergotamine and methysergide, and uses of antihypertensive drugs such as guanfacine.

Kidney fibrosis or renal fibrosis is a characteristic feature of most forms of chronic kidney diseases (CKD). Deposition of pathological fibrillar matrix rich in fibrillar collagen I and III in the interstitial space and within the walls of glomerular capillaries as well as the cellular processes resulting in this deposition are increasingly recognized as important factors amplifying kidney injury and accelerating nephron demise. Both clinical and subclinical insults contribute to kidney fibrosis and CKD development, including infections, xenobiotics, toxins, mechanical obstruction, immune complexes resulting from autoimmune diseases or chronic infections (infectious glomerulonephritis), renal vasculitis, ureteral obstruction, and genetic disorders. The most common causes of CKD in developed nations are, however, type-2 diabetes mellitus and ischemic/hypertensive nephropathy, which frequently coexist in the same kidney or complicate other diseases. In an embodiment, a compound or composition disclosed herein prevents or treats glomerulosclerosis and tubulointerstitial fibrosis.

Bone marrow fibrosis (BMF) is a central pathological feature of myelofibrosis. BMF is characterized by the increased deposition of reticulin fibers and in some cases collagen fibers. There are a number of hematologic and non-hematologic disorders that are associated with increased BMF including myeloproliferative disorders (several types of leukemias, lymphomas, myelomas) as well as other diseases such as HIV infection, visceral leishmaniasis, systemic mastocytosis, myelodysplastic syndromes and osteopetrosis (see, e.g., Zahr et al., Haematologica. 2016 June; 101(6): 660-671). Myeloproliferative disorders are associated with bone marrow fibrosis and erythropoiesis failure resulting in extramedullary haematopoiesis (Stem Cell Investig 3 (5) 1-10, 2016). Myelofibrosis (MF) is a fatal disorder of the bone marrow which disturbs the normal production of the blood cells in the body. This results in massive scarring in the bone marrow leading to severe anemia, fatigue, weakness and usually an enlarged liver and spleen.

In another aspect, the present disclosure relates to a method for treating hypertension (reducing blood pressure) in a subject in need thereof comprising administering an effective amount of a compound of formula (I), salt thereof or composition disclosed herein. The present disclosure also relates to the use of a compound of formula (I), salt thereof or composition disclosed herein for treating hypertension (reducing blood pressure) in a subject, or for the manufacture of a medicament for treating hypertension (reducing blood pressure) in a subject. The present disclosure also relates to a compound of formula (I), salt thereof or composition disclosed herein for use in treating hypertension (reducing blood pressure) in a subject.

Long-term high blood pressure is a major risk factor for coronary artery disease, stroke, heart failure, atrial fibrillation, peripheral arterial disease, vision loss, chronic kidney disease, and dementia. Thus, in embodiments, the method for treating hypertension disclosed herein reduces the risk that the subject suffers from coronary artery disease (CAD), stroke, heart failure, atrial fibrillation, peripheral arterial disease (PAD), vision loss, chronic kidney disease (CKD), and/or dementia.

In an embodiment, the hypertension is secondary hypertension associated with a kidney disease/condition such as CKD or renal artery stenosis (from atherosclerosis or fibromuscular dysplasia).

In another aspect, the present disclosure relates to a method for treating cancer in a subject in need thereof comprising administering an effective amount of a compound of formula (I), salt thereof or composition disclosed herein. The present disclosure also relates to the use of a compound of formula (I), salt thereof or composition disclosed herein for treating cancer in a subject, or for the manufacture of a medicament for treating cancer in a subject. The present disclosure also relates to a compound of formula (I), salt thereof or composition disclosed herein for use in treating cancer in a subject.

In an embodiment, the cancer is one of the twelve major cancers, i.e. prostate, breast, lung, colorectal, bladder, non-Hodgkin's lymphoma, uterine, melanoma, kidney, leukemia, ovarian, or pancreatic cancer. In an embodiment, the method is for the treatment of a primary tumor. In another embodiment, the method is for preventing or treating tumor metastasis.

In another aspect, the present disclosure relates to a method for stimulating or activating the GPR40 and/or GPR120 receptor (e.g., for stimulating or activating a GPR40- and/or GPR120-associated pathway) in a cell comprising contacting the cell with a compound of formula (I), salt thereof or composition disclosed herein. The present disclosure also relates to the use of a compound of formula (I), salt thereof or composition disclosed herein for stimulating or activating the GPR40 and/or GPR120 receptor (e.g., for stimulating or activating a GPR40- and/or GPR120-associated pathway) in a cell. The present disclosure also relates to a compound of formula (I), salt thereof or composition disclosed herein for use in stimulating or activating the GPR40 and/or GPR120 receptor (e.g., for stimulating or activating a GPR40- and/or GPR120-associated pathway) in a cell.

GPR40 (Free Fatty Acid Receptor 1, FFAR1) potentiates glucose-dependent insulin secretion and demonstrated in clinical studies robust glucose lowering in type 2 diabetes, and GPR120 (Free Fatty Acid Receptor 4, FFAR4) has been shown to improve insulin sensitivity. Activation of GPR40 and GPR120 has been shown to modulate both adipose tissue lipolysis and glucose metabolism, highlighting the strong potential of these receptors in fatty acid and glucose metabolism (Satapati et al., J Lipid Res. 2017; 58(8):1561-1578. Epub 2017 Jun. 5). Thus, in another aspect, the present disclosure relates to a method for preventing or treating a metabolic condition (e.g., a condition related to dysregulated fatty acid and/or glucose metabolism) in a subject in need thereof comprising administering an effective amount of a compound of formula (I), salt thereof or composition disclosed herein. The present disclosure also relates to the use of a compound of formula (I), salt thereof or composition disclosed herein for preventing or treating a metabolic condition (e.g., a condition related to dysregulated fatty acid and/or glucose metabolism) in a subject, or for the manufacture of a medicament for preventing or treating a metabolic condition in a subject. The present disclosure also relates to a compound of formula (I), salt thereof or composition disclosed herein for use in preventing or treating a metabolic condition in a subject. The term “metabolic condition” as used herein refers to a disease, condition or disorder associated with a dysregulation of the metabolism of lipids, fatty acids and/or carbohydrates (e.g., glucose). In an embodiment, the metabolic condition is metabolic syndrome, pre-diabetes (e.g., insulin resistance, glucose intolerance), diabetes, hyperinsulinemia, dyslipidemia (e.g., hyperlipidemia, hypertriglyceridemia, hypercholesterolemia), or obesity. In a further embodiment, the metabolic condition is pre-diabetes (e.g., insulin resistance, glucose intolerance) or diabetes. The term “diabetes” includes Type I diabetes, Type II diabetes, Type III diabetes (Alzheimer), maturity-onset diabetes of the young, latent autoimmune diabetes of adults (LADA), and gestational diabetes. In an embodiment, the diabetes is Type II diabetes.

In another aspect, the present disclosure relates to a method for inhibiting or antagonizing the GPR84 receptor (e.g., for inhibiting or reducing a GPR84-associated pathway) in a cell comprising contacting the cell with a compound of formula (I), salt thereof or composition disclosed herein. The present disclosure also relates to the use of a compound of formula (I), salt thereof or composition disclosed herein for inhibiting the GPR84 receptor (e.g., for inhibiting or reducing a GPR84-associated pathway) in a cell. The present disclosure also relates to a compound of formula (I), salt thereof or composition disclosed herein for use in inhibiting the GPR84 receptor (e.g., for inhibiting or reducing a GPR84-associated pathway) in a cell.

GPR84 (also referred to as Inflammation-related G-protein coupled receptor EX33) is often described as a pro-inflammatory receptor and is expressed by a range of immune cell types. GPR84 is upregulated on both macrophages and neutrophil granulocytes after LPS stimulation and infections. There is evidence that GPR84 blockade may be effective in idiopathic pulmonary fibrosis and other fibrotic indications, as well as in the treatment of autoimmune or inflammatory conditions such as ulcerative colitis and atherosclerosis (Gagnon, L. et al. Am J Pathol. 188, 1132-1148 (2018); Vermeire, S. et al. J Crohn's Colit. 11 Issue suppl_1, S390-S391 (2017); Gaidarov, I. et al. Pharmacol Res. 131, 185-198 (2018)).

Thus, in another aspect, the present disclosure relates to a method for reducing inflammation in an organ and/or tissue of a subject in need thereof comprising administering an effective amount of a compound of formula (I), salt thereof or composition disclosed herein. The present disclosure also relates to the use of a compound of formula (I), salt thereof or composition disclosed herein for reducing inflammation in an organ and/or tissue of a subject, or for the manufacture of a medicament for reducing inflammation in an organ and/or tissue of a subject. The present disclosure also relates to a compound of formula (I), salt thereof or composition disclosed herein for use in reducing inflammation in an organ and/or tissue of a subject. Such inflammation may be caused by an injury to the tissue or organ, e.g., due to trauma, microbial invasion, or noxious compounds (acute inflammation), or to more chronic agents such as chronic infections, chronic exposure to an irritant or foreign material, autoimmune disorders such as rheumatoid arthritis (RA), systemic lupus erythematosus (SLE), defects in the cells responsible for mediating inflammation leading to persistent or recurrent inflammation, inflammatory inducers causing oxidative stress and mitochondrial dysfunction such as increased production of free radical molecules, advanced glycation end products (AGEs), uric acid (urate) crystals, and oxidized lipoproteins, for example (chronic inflammation). Chronic inflammation occurs in several diseases and disorders including cardiovascular diseases (e.g., atherosclerosis), diabetes, rheumatoid arthritis, allergic asthma, chronic obstructive pulmonary disease (COPD), Alzheimer's disease, chronic kidney disease (CKD), inflammatory Bowel Disease (IBD).

Thus, in another aspect, the present disclosure relates to a method for preventing or treating an inflammatory or autoimmune condition in a subject in need thereof comprising administering an effective amount of a compound of formula (I), salt thereof or composition disclosed herein. The present disclosure also relates to the use of a compound, salt thereof or composition disclosed herein for preventing or treating an inflammatory or autoimmune condition in a subject, or for the manufacture of a medicament for preventing or treating an inflammatory or autoimmune condition in a subject. The present disclosure also relates to a compound, salt thereof or composition disclosed herein for use in preventing or treating an inflammatory or autoimmune condition in a subject. The term “inflammatory or autoimmune condition” as used herein refers to a disease, condition or disorder in which a dysregulated immune response or inflammatory reaction leads to tissue or organ damages. Examples of inflammatory or autoimmune condition include arthritis, glomerulonephritis, atherosclerosis, vasculitis, arthritis, systemic lupus erythematoses (SLE), idiopathic thrombocytopenic purpura (ITP), psoriasis, inflammatory bowel diseases (e.g., Crohn's disease), ankylosing spondylitis, Sjogren's syndrome, Still's disease (macrophage activation syndrome), uveitis, scleroderma, myositis, Reiter's syndrome, Wegener's syndrome, and multiple sclerosis.

A compound of formula (I) or salt thereof or composition disclosed herein may be used alone or in combination with other therapies for the treatment of the above-noted disease or condition.

In an embodiment, the above-mentioned treatment comprises the use/administration of more than one (i.e. a combination of) active/therapeutic agent or therapy, one of which being the above-mentioned compound of formula I or salt thereof. The combination of therapeutic agents or therapies may be administered or co-administered (e.g., consecutively, simultaneously, at different times) in any conventional manner. Co-administration in the context of the present disclosure refers to the administration of more than one therapy in the course of a coordinated treatment to achieve an improved clinical outcome. Such co-administration may also be coextensive, that is, occurring during overlapping periods of time. For example, a first therapy may be administered to a patient before, concomitantly, before and after, or after a second therapy is administered. In the case of a combination of active agents, they may be combined/formulated in a single composition and thus administered at the same time.

In an embodiment, the compound of formula I or salt thereof is used in combination with one or more therapies for the treatment of anemia and/or leukopenia, i.e. iron supplementation, blood transfusion, folic acid supplementation, erythropoietin (EPO) and growth factors (e.g., G-CSF).

In an embodiment, the compound of formula I or salt thereof is used in combination with one or more therapies for the treatment of one or more symptoms of fibrosis.

In an embodiment, the compound of formula I or salt thereof is used in combination with one or more therapies for the treatment of hypertension. Several classes of medications, collectively referred to as antihypertensive medications, are available for treating hypertension. First-line medications for hypertension include thiazide-diuretics, calcium channel blockers, angiotensin converting enzyme inhibitors (ACE inhibitors), and angiotensin receptor blockers (ARBs).

In an embodiment, the compound of formula I or salt thereof is used in combination with one or more therapies for the treatment of cancer. Generally, four types of treatment have been used for the treatment of metastatic cancers: surgery, radiation therapy, chemotherapy, and immunotherapy.

### MODE(S) FOR CARRYING OUT EMBODIMENTS OF THE DISCLOSURE

The present disclosure is illustrated in further details by the following non-limiting examples.

### Example 1: Synthesis of Compounds

All HPLC chromatograms and mass spectra were recorded on an HP 1100 LC-MS Agilent instrument using an analytical C18 column (250×4.6 mm, 5 microns) with a gradient over 5 min of 15-99% acetonitrile-water with 0.01% trifluoroacetic acid as the eluant and a flow of 2 mL/min.

### General Scheme for the Preparation of 2-cyclopropylacetate Compounds

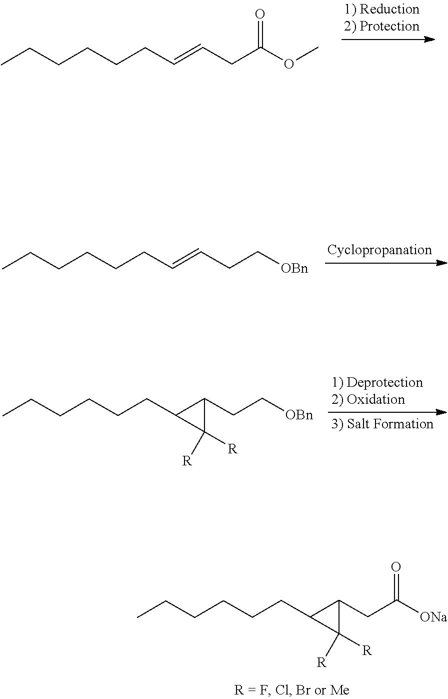

### Compound I: Synthesis of sodium salt of 2-(2,2-difluoro-3-hexylcyclopropyl)acetic acid

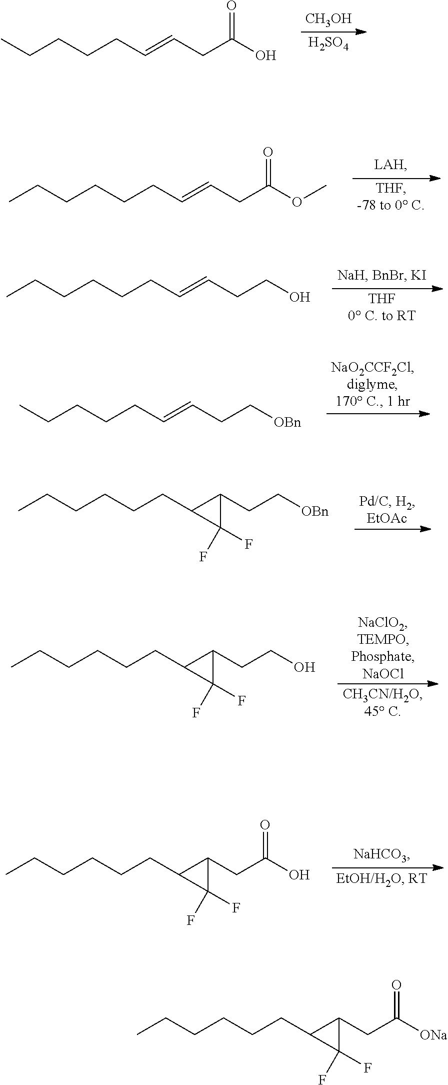

Step 1: 3-Decenoic acid (10 g, 58.7 mmol) was dissolved in methanol (100 mL) at room temperature. Concentrated sulfuric acid (0.5 mL) was added and the reaction was stirred for 16 hrs. A solution of saturated sodium bicarbonate (100 mL) was added and the mixture was extracted three times with ethyl acetate. The organic layers were combined, washed with brine and dried over anhydrous sodium sulfate. Concentration of the solution in vacuo gave methyl (E)-dec-3-enoate as a faintly yellow oil (10.2 g, 97%). ¹H NMR (400 MHz, CDCl₃) δ 5.46-5.59 (m, 2H), 3.67 (s, 3H), 3.02 (m, 2H), 2.00 (m, 2H), 1.23-1.36 (m, 8H), 0.86 (t, J=7 Hz, 3H).

Step 2: Methyl (E)-dec-3-enoate (30.0 g, 163 mmol) was dissolved in dry tetrahydrofuran (350 mL) and cooled to −78° C. Lithium aluminium hydride (8.0 g, 212 mmol) was then added in three portions over fifteen minutes. Once the addition was completed, the reaction was stirred at −78° C. for thirty minutes. The reaction was then warmed to 0° C. and stirred for an additional thirty minutes. Ethyl acetate (10 mL) was added to quench the reaction mixture followed by a half-saturated solution of Rochelle's salt (150 mL). More ethyl acetate was then added and the mixture was warmed to room temperature and stirred vigorously for several hours. The aqueous layer was extracted three times with ethyl acetate. Organic layers were combined, washed with brine and dried over sodium sulfate. Evaporation of the solvent to dryness gave (E)-dec-3-en-1-ol as colorless oil (26.0 g, 99%). ¹H NMR (400 MHz, CDCl₃) δ 5.50-5.58 (m, 1H), 5.32-5.40 (m, 1H), 3.60 (t, J=6 Hz, 2H), 2.22-2.27 (m, 2H), 2.00-2.03 (m, 2H), 1.67 (bs, 1H), 1.22-1.35 (m, 8H), 0.87 (t, J=7 Hz, 3H).

Step 3: (E)-Dec-3-en-1-ol (25.8 g, 167 mmol) was dissolved in dry tetrahydrofuran (500 mL) and cooled to 0° C. Sodium hydride (60 wt % oil dispersion, 13.4 g, 335 mmol) was added portion-wise over ten minutes and once the addition was completed the reaction was stirred for 20 minutes. Potassium iodide (11.1 g, 67 mmol) was then added followed by benzyl bromide (40 mL, 335 mmol). The reaction was allowed to warm to room temperature and then stirred for 16 hrs. Then water was added and the mixture was extracted three times with ethyl acetate. Organic layers were combined, washed with brine and dried over sodium sulfate. Evaporation of the solvent to dryness followed by purification on silica gel (0-10% diethyl ether in Hexanes) gave (E)-((dec-3-en-1-yloxy)methyl)benzene (34.5 g, 85%). ¹H NMR (400 MHz, CDCl₃) δ 7.26-7.38 (m, 5H), 5.40-5.53 (m, 2H), 4.52 (s, 2H), 3.48 (t, J=7 Hz, 2H), 2.30-2.35 (m, 2H), 1.99 (q, J=7 Hz, 2H), 1.25-1.36 (m, 8H), 0.89 (t, J=7 Hz, 3H).

Step 4: A solution of (E)-((dec-3-en-1-yloxy)methyl)benzene (8.0 g, 32.8 mmol) in diglyme (100 mL) was heated to reflux and sodium difluorochloroacetate (24.9 g, 164 mmol) was added portion-wise over 30 minutes. Once the addition was completed, refluxing was continued for additional 30 minutes then the reaction mixture was cooled to room temperature. The mixture was diluted with water (100 mL) and extracted four times with hexanes. The organic layers were combined, washed with brine and dried over sodium sulphate. Concentration of the solution in vacuo gave an oil which was purified on silica gel (0-10% diethyl ether in hexanes) and on HPLC (80-100% acetonitrile+0.1% trifluoroacetic acid in water+0.1% trifluoroacetic acid) to give ((2-(2,2-difluoro-3-hexylcyclopropyl)ethoxy)methyl)benzene as a yellow oil (6.9 g, 72%). ¹H NMR (400 MHz, CDCl₃) δ 7.26-7.38 (m, 5H), 4.52 (dd, J=12, 2 Hz, 2H), 3.53, (t, J=6 Hz, 2H), 1.81 (sextet, J=7 Hz, 1H), 1.64-1.71 (m, 1H), 1.19-1.49 (m, 11H), 1.11 (sextet, J=7, 1H), 0.88 (t, J=7 Hz, 3H); ¹⁹F NMR (376.5 MHz, CDCl₃): δ −139.3 (qd, 2F, J=155.15 Hz).

Step 5: To a degassed solution of ((2-(2,2-difluoro-3-hexylcyclopropyl)ethoxy)methyl)benzene (6.9 g, 23.2 mmol) in ethyl acetate (50 mL), was added Pd/C (10 wt % Pd, 1.0 g). Nitrogen gas was bubbled for five minutes. Reaction was then sealed and hydrogen was introduced via balloon. After bubbling hydrogen into the reaction mixture for several minutes, the reaction was left to stir under hydrogen atmosphere for 16 hrs. The reaction was then opened to air and filtered through Celite™. Concentration of the solution in vacuo gave 2-(2,2-difluoro-3-hexylcyclopropyl)ethan-1-ol as a colorless oil (4.9 g, 99%). ¹H NMR (400 MHz, CDCl₃) δ 3.70 (td, 2H, J=6, 1 Hz), 1.67-1.74 (m, 2H), 1.25-1.50 (m, 10H), 1.10-1.23 (m, 2H), 0.88 (t, 3H, 7 Hz); ¹³C NMR (125 MHz, CDCl₃) δ 116 (t, J=289 Hz), 61.9, 31.7, 29.9, 28.8, 28.7, 28.3, 26.5, 25.1, 22.6, 14.1; ¹⁹F NMR (376.5 MHz, CDCl₃): δ −138.1 (qd, 2F, J=154.15 Hz).

Step 6: To a solution of 2-(2,2-difluoro-3-hexylcyclopropyl)ethan-1-ol (4.9 g, 23.7 mmol) in acetonitrile/water (75 ml/15 mL) were added monosodium phosphate (5.0 g), sodium chlorite (4.2 g, 47.4 mmol) and 2,2,6,6-tetramethylpiperidine 1-oxyl (TEMPO, 0.19 g, 1.19 mmol). The reaction was then heated to 45° C. and sodium hypochlorite (10-15% aqueous solution) was added dropwise over two hours until the reaction remained yellow (took 10.5 mL of solution). The reaction mixture was then diluted with hydrochloric acid (0.1 M, 50 mL) and extracted three times with ethyl acetate. Organic layers were combined, washed with brine and dried over sodium sulphate. Concentration of the solution in vacuo gave 2-(2,2-difluoro-3-hexylcyclopropyl)acetic acid as a colorless oil (5.12 g, 98%)) which required no further purification. ¹H NMR (400 MHz, CDCl₃) δ 11.4 (bs, 1H), 2.55-2.62 (m, 1H), 2.42-2.49 (m, 1H), 1.19-1.51 (m, 12H), 0.88 (t, J=7 Hz, 3H); ¹³C NMR (125 MHz, CDCl₃) δ 180.0, 38.9, 33.8, 31.9, 29.3, 29.1, 22.6, 18.6, 14.1, 13.9, 11.6; ¹⁹F NMR (376.5 MHz, CDCl₃): δ −139.6 (qd, 2F, J=156, 14 Hz).

Step 7: To a stirred solution of 2-(2,2-difluoro-3-hexylcyclopropyl)acetic acid (5.12 g, 23.3 mmol) in ethanol/water (40 mL/10 mL) was added sodium bicarbonate (2.0 g, 23.3 mmol) at room temperature and the reaction was stirred for 16 hrs. Reaction mixture was then concentrated in vacuo and dried. Trituration with n-Butyl acetate followed by lyophilization of this material gave sodium 2-(2,2-difluoro-3-hexylcyclopropyl)acetate as a fluffy white solid (4.5 g, 81%). ¹H NMR (400 MHz, CD₃OD) δ 2.32 (m, 1H), 2.20 (m, 1H), 1.29-1.89 (m, 11H), 1.20 (m, 1H), 0.90 (t, J=7 Hz, 1H); ¹³C NMR (125 MHz, CD₃OD) δ 178.6, 116.3 (t, J=288 Hz), 34.8, 31.5, 28.5, 28.0, 27.9, 26.4, 25.6, 22.3, 13.0; ¹⁹F NMR (376.5 MHz, CD₃OD) −140.8 (m); LRMS (ESI): m/z (M−) 220.1, HPLC: 1.9 min.

### Compound II: Synthesis of sodium salt of 2-(2,2-dibromo-3-hexylcyclopropyl)acetate

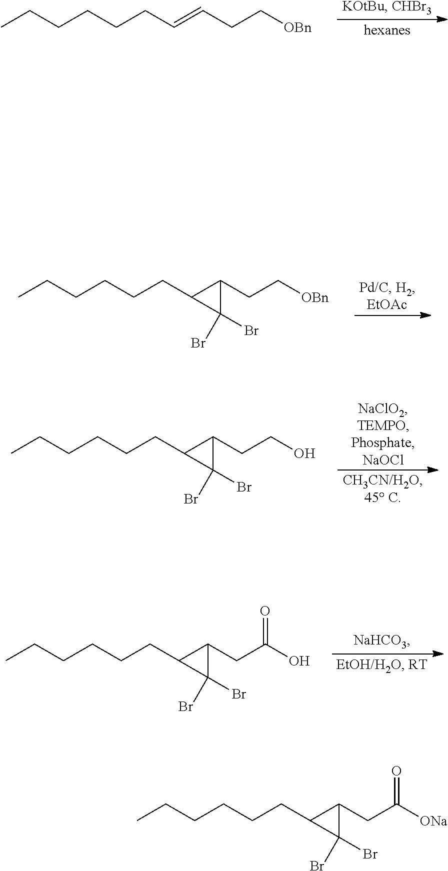

Step 1: Bromoform (25.0 mL, 278 mmol) was added dropwise to a slurry of (E)-((dec-3-en-1-yloxy)methyl)benzene (17.0 g, 69.7 mmol) and n-butyl tert-butoxide (31.2 g, 278 mmol) in hexanes over 1 hr at 0° C. After the addition was completed, the reaction was warmed to room temperature and stirred for an additional hour. The reaction is then diluted with water and extracted two times with diethyl ether. The organic layers are combined, washed with brine and dried over sodium sulphate. Concentration of the solvent in vacuo gave ((2-(2,2-dibromo-3-hexylcyclopropyl)ethoxy)methyl)benzene (23.8 g, 82%) as brown oil. ¹H NMR (400 MHz, CDCl₃) δ 7.25-7.40 (m, 5H), 4.54 (s, 2H), 3.61 (m, 2H), 1.94 (m, 1H), 1.74 (m, 1H), 1.61 (m, 1H), 1.25-1.50 (m, 10H), 1.13 (m, 1H), 0.89 (t, J=7 Hz, 1H).

Step 2: 2-(2,2-Dibromo-3-hexylcyclopropyl)ethan-1-ol was prepared as for compound I step 5 by hydrogenation of ((2-(2,2-dibromo-3-hexylcyclopropyl) ethoxy)methyl)benzene. ¹H NMR (400 MHz, CDCl₃) δ 3.74 (t, J=6 Hz, 2H), 1.91 (bs, 1H), 1.82 (m, 1H), 1.66 (m, 1H), 1.56 (m, 1H), 1.33-1.45 (m, 3H), 1.15-1.31 (m, 7H), 0.83 (t, J=7 Hz, 3H).

Step 3: 2-(2,2-Dibromo-3-hexylcyclopropyl)acetic acid was prepared as for compound I step 6 by oxidizing 2-(2,2-Dibromo-3-hexylcyclopropyl)ethan-1-ol. ¹H NMR (400 MHz, CDCl₃) δ 2.71 (dd, J=18, 7 Hz, 1H), 2.48 (dd, J=18, 7 Hz, 1H), 1.38-1.56 (m, 4H), 1.16-1.31 (m, 8H), 0.82 (t, J=7 Hz, 3H).

Step 4: Sodium 2-(2,2-dibromo-3-hexylcyclopropyl)acetate was prepared as for compound I step 7 by basic treatment of 2-(2,2-dibromo-3-hexylcyclopropyl)acetic acid. Mp 108-111° C., ¹H NMR (400 MHz, CD₃OD) δ 2.56 (dd, J=15, 6 Hz, 1H), 2.21 (dd, J=15, 6 Hz, 1H), 1.47-1.59 (m, 5H), 1.28-1.40 (m, 6H), 1.20 (q, J=7 Hz, 1H), 0.91 (t, J=7 Hz, 3H); ¹³C NMR (125 MHz, CD₃OD) δ 178.4, 41.1, 38.5, 37.1, 34.3, 32.6, 31.8, 29.0, 28.1, 22.5, 13.3, LRMS (ESI): m/z (M−) 339, HPLC: 7.1 min.

### Compound III: Synthesis of sodium salt of 2-(3-hexyl-2,2-dimethylcyclopropyl)acetate

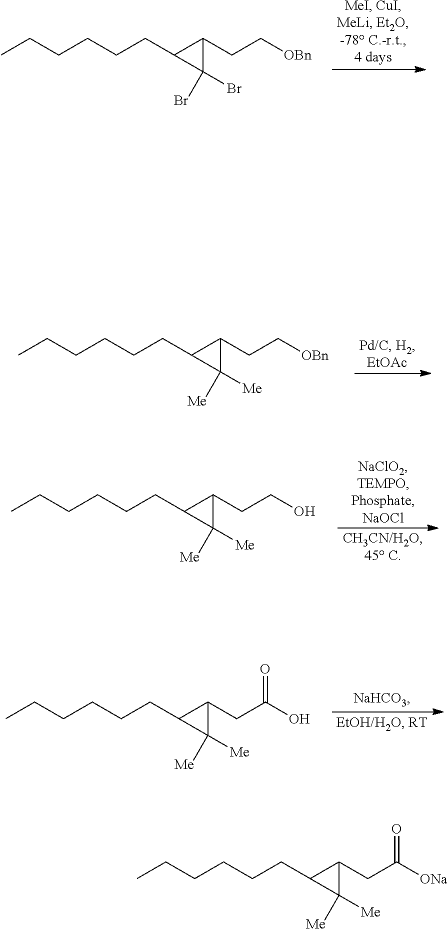

Step 1: A solution of methyl lithium (458 mmol, 3.1 M in 1,2-dimethoxyethane) was added to a suspension of flame-dried copper iodide in tetrahydrofuran at −78° C. This stirred mixture was allowed to slowly warm to 0° C. until the solution became homogeneous (approx. five minutes) then recooled to −78° C. A solution of ((2-(2,2-dibromo-3-hexylcyclopropyl)ethoxy)methyl)benzene (12.0 g, 28.6 mmol) in ether (25 mL) was then added dropwise over 20 minutes and the resultant solution was stirred at 0° C. for 48 hrs. Methyl iodide was then added and the mixture was stirred at room temperature for an additional 24 hours. The reaction was then quenched with saturated solution of ammonium chloride and extracted three times with diethyl ether. Organic layers were combined, washed with brine and dried over sodium sulphate. Concentration of the solvent in vacuum gave a colorless oil that was purified on silica gel (0-10% diethyl ether in hexanes) followed by further purification using HPLC (80-100% acetonitrile+0.1% trifluoroacetic acid in water+0.1% trifluoroacetic acid) to give ((2-(3-hexyl-2,2-dimethylcyclopropyl)ethoxy)methyl)benzene as a colorless oil (8.0 g, 82%). ¹H NMR (400 MHz, CDCl₃) δ 7.26-7.37 (m, 5H), 4.52 (s, 2H), 3.50 (t, J=7 Hz, 2H), 1.70 (m, 1H), 1.52 (m, 1H), 1.14-1.33 (m, 10H), 0.99 (d, J=2 Hz, 6H), 0.88 (t, J=7 Hz, 3H), 0.09-0.18 (m, 2H); ¹³C NMR (125 MHz, CDCl₃) δ 138.7, 128.3, 127.6, 127.4, 72.9, 71.0, 31.9, 30.8, 30.2, 29.9, 29.4, 29.3, 27.3, 22.7, 22.1, 21.8, 18.9, 14.1.

Step 2: 2-(3-Hexyl-2,2-dimethylcyclopropyl)ethan-1-ol was prepared as for compound I step 5 by hydrogenation of ((2-(3-hexyl-2,2-dimethylcyclopropyl)ethoxy)methyl)benzene. ¹H NMR (400 MHz, CDCl₃) δ 3.66 (t, J=7 Hz, 2H), 1.66 (m, 1H), 1.52 (s, 1H), 1.46 (m, 1H), 1.19-1.34 (m, 10H), 1.01 (d, J=2 Hz, 6H), 0.88 (t, J=7 Hz, 3H), 0.08-0.17 (m, 2H); ¹³C NMR (125 MHz, CDCl₃) δ 63.6, 32.7, 31.9, 30.7, 30.2, 29.4, 29.3, 27.0, 22.7, 22.2, 21.8, 18.7, 14.1.

Step 3: 2-(3-Hexyl-2,2-dimethylcyclopropyl)acetic acid was prepared as for compound I step 6 by oxidizing 2-(3-hexyl-2,2-dimethylcyclopropyl)ethan-1-ol. ¹H NMR (400 MHz, CDCl₃) δ 2.35 (dd, J=7, 1 Hz, 1H), 1.26-1.33 (m, 10H), 1.03 (d, J=6 Hz, 6H), 0.87 (t, J=7 Hz, 3H), 0.49 (m, 1H), 0.23 (m, 1H); ¹³C NMR (125 MHz, CDCl₃) δ 180.3, 34.5, 31.9, 20.8, 29.9, 29.2, 29.1, 25.6, 22.7, 22.1, 21.3, 19.1, 14.1.

Step 4: Sodium 2-(3-hexyl-2,2-dimethylcyclopropyl)acetate was prepared as for compound I step 7 by basic treatment of 2-(3-hexyl-2,2-dimethylcyclopropyl)acetic acid. ¹H NMR (400 MHz, CD₃OD) δ 2.17 (dd, J=14, 7 Hz, 1H), 2.10 (dd, J=14, 7 Hz, 1H), 1.28-1.37 (m, 10H), 1.02 (d, J=2 Hz, 6H), 0.89 (t, J=7 Hz, 3H), 0.55 (m, 1H), 0.19 (m, 1H); ¹³C NMR (125 MHz, CD₃OD) δ 181.7, 37.9, 31.7, 30.6, 29.9, 29.2, 29.1, 27.6, 22.3, 21.1, 20.6, 18.4, 13.1.

### Compound IV: Synthesis of sodium salt of 2-(2-hexylcyclopropyl)-2-oxoacetate

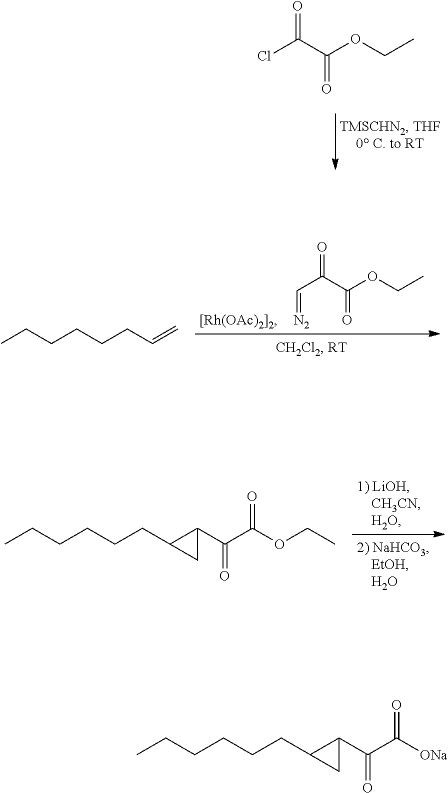

Step 1: Oct-1-ene (5.0 g, 44.1 mmol) was dissolved in dry dichloromethane (100 mL) and degassed with Argon. Rhodium(II) acetate (0.2 g, 0.44 mmol) was then added and degassing was continued for several minutes. Reaction was then sealed and a solution of ethyl 3-diazooxopropanate (3.1 g, 22.0 mmol) in dichloromethane (25 mL) was added dropwise under argon atmosphere over 4 hrs via syringe pump. Once the addition was completed, the reaction was stirred at room temperature for 16 hrs. The mixture was then filtered through Celite™ and concentrated in vacuo to give a green oil which was purified on silica gel (0-10% ethyl acetate in hexanes) to obtain pure ethyl 2-(2-hexylcyclopropyl)-2-oxoacetate as a yellow oil (2.4 g, 50%).

¹H NMR (400 MHz, CDCl₃) δ 4.28-4.35 (isomer A/B, m, 2H), 2.81 (isomer A, m, 1H), 2.50 (isomer B, m, 1H), 1.43-1.66 (isomer A/B, 2H), 1.34-1.38 (isomer A/B, m, 3H) 1.21-1.32 (isomer A/B, m, 10H), 0.99-1.04 (isomer A, m, 1H), 0.85 (isomer A/B, q, 3H, J=7 Hz); ¹³C NMR (125 MHz, CDCl₃) δ 193.6, 192.5, 161.5, 161.1, 62.4, 62.3, 33.3, 31.7, 30.4, 29.8, 29.7, 29.0, 28.9, 28.8, 26.0, 25.9, 23.5, 22.6, 22.5, 21.3, 17.5, 14.1, 14.0.

Step 2; Ethyl 2-(2-Hexylcyclopropyl)-2-oxoacetate (2.0 g, 8.8 mmol) was dissolved in acetonitrile/H₂O (50/10 mL) at room temperature and lithium hydroxide (1.1 g, 44.2 mmol) was added. The reaction was stirred for 18 hours, then diluted with HCl (0.1 M) solution and extracted three times with ethyl acetate. The organic layers were combined, washed with brine and dried over sodium sulfate. Concentration of the solvent in vacuo gave 2-(2-Hexylcyclopropyl)-2-oxoacetic acid as a colorless oil (1.55 g, 89%) that was used without further purification. ¹H NMR (400 MHz, CDCl₃) δ 3.08 (isomer A, multiplet, 1H), 2.74 (isomer B, m, 1H), 1.86 (isomer A, m, 1H), 1.70 (isomer B, m, 1H), 1.56 (isomers A/B, m, 1H), 1.10-1.47 (isomers A/B, m, 11H), 0.86 (m, 3H).

Step 3; Sodium 2-(2-hexylcyclopropyl)-2-oxoacetate was prepared as for compound I step 7 by basic treatment of 2-(2-hexylcyclopropyl)-2-oxoacetic acid. Mp 152-254° C., ¹H NMR (400 MHz, CD₃OD) δ 2.70 (isomer A, m, 1H), 2.29 (isomer B, m, 1H), 1.24-1.55 (isomer A/B, m, 12H), 1.12 (isomer A, m, 1H), 1.04 (isomer A, m, 1H), 0.90 (m, 3H); ¹³C NMR (125 MHz, CD₃OD) δ 204.7, 203.5, 169.6, 33.0, 31.6, 31.5, 29.5, 28.8, 28.7, 27.3, 27.0, 26.0, 25.5, 22.9, 22.3, 22.2, 18.4, 15.1, 13.0.

### Compound V: Synthesis of sodium 1-octylcyclopropanecarboxylate

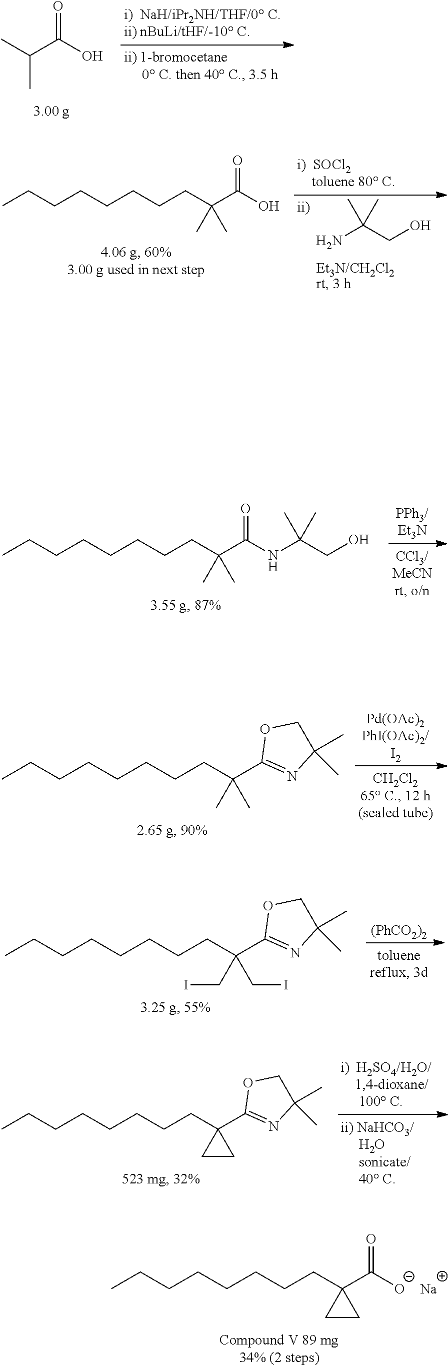

Step 1: A suspension of sodium hydride (60% dispersion in oil, 1.50 g, 37.4 mmol) in anhydrous tetrahydrofuran (15 ml), was cooled to 0° C. under nitrogen, and was then treated dropwise with diisopropylamine (4.86 ml, 34.7 mmol), followed by a solution of 2-methylpropanoic acid (3.00 g, 34.0 mmol) in anhydrous tetrahydrofuran (5 ml). The reaction was stirred for 10 min at 0° C., for 10 min at ambient temperature, for 30 min at reflux, then cooled to −10° C. A solution of n-butyllithium in hexanes (1.5M, 22.7 ml, 34.0 mmol) was added dropwise, and the reaction was stirred for 15 min at 0° C., for 30 min at 40° C., then cooled to 0° C. A solution of 1-bromooctane (6.22 ml, 35.8 mmol) in anhydrous tetrahydrofuran (5 ml), was added dropwise at 0° C., and the reaction was then stirred for 15 min at 0° C., then for 3.5 h at 40° C. After cooling to ambient temperature, the reaction was quenched with water, then diluted with water and washed with ethyl acetate. The aqueous phase was then acidified with 1M aqueous hydrochloric acid and extracted with ethyl acetate. The organic extract was dried over magnesium sulfate; filtered and evaporated in vacuo to give 2,2-dimethyldecanoic acid (4.06 g, 60%), as a pale yellow oil. ¹H NMR (400 MHz, CDCl₃): δ 11.95 (br s, 1H), 1.50-1.55 (m, 2H), 1.23-1.32 (m, 12H), 1.18 (s, 6H), 0.87 (t, J=6.9 Hz, 3H).

Step 2: A solution of 2,2-dimethyldecanoic acid (3.00 g, 15.0 mmol) in toluene (15 ml), was treated with thionyl chloride (3.28 ml, 45.0 mmol), and the reaction was stirred at 80° C. for 1 h. Solvents were evaporated in vacuo, and the residue was dissolved in anhydrous dichloromethane (15 ml). The solution was cooled to 0° C., and was treated with triethylamine (2.51 ml, 18.0 mmol) and with 2-amino-2-methyl-1-propanol (1.57 ml, 16.5 mmol). The reaction was stirred at ambient temperature for 3.25 h, then was partitioned between ethyl acetate and 1M aqueous hydrochloric acid. The organic phase was washed with saturated aqueous sodium bicarbonate, and with saturated aqueous sodium chloride; then dried over magnesium sulfate; filtered and evaporated in vacuo to give the crude product. Purification by silica gel chromatography, eluting with 5 to 15% ethyl acetate in hexanes gave N-[1-hydroxy-2-methylpropan-2-yl]-2,2-dimethyldecanamide (3.55 g, 87%), as a pale yellow oil. ¹H NMR (400 MHz, CDCl₃): δ 5.60 (br s, 1H), 5.17 (t, J=5.9 Hz, 1H), 3.55 (d, J=5.7 Hz, 2H), 1.43-1.47 (m, 2H), 1.27 (s, 6H), 1.16-1.31 (m, 12H), 1.13 (s, 6H), 0.86 (t, J=6.9 Hz, 3H).

Step 3: A solution of N-[1-hydroxy-2-methylpropan-2-yl]-2,2-dimethyldecanamide (3.52 g, 13.0 mmol) in triethylamine (28 ml), carbon tetrachloride (28 ml) and acetonitrile (100 ml), was treated with triphenylphosphine (13.6 ml, 51.8 mmol), and the reaction was stirred at ambient temperature overnight. The reaction mixture was diluted with ethyl acetate, then washed with saturated aqueous sodium bicarbonate; dried over magnesium sulfate; filtered and evaporated in vacuo to give the crude product. Purification by silica gel chromatography, eluting with 2 to 10% ethyl acetate in hexanes gave 4,4-dimethyl-2-[2-methyldecan-2-yl]-4,5-dihydrooxazole (2.65 g, 90%), as a pale yellow oil. ¹H NMR (400 MHz, CDCl₃): δ 3.86 (s, 2H), 1.44-1.48 (m, 2H), 1.24 (s, 6H), 1.18-1.30 (m, 12H), 1.16 (s, 6H), 0.86 (t, J=6.9 Hz, 3H).

Step 4: A solution of 4,4-dimethyl-2-[2-methyldecan-2-yl]-4,5-dihydrooxazole (2.64 g, 11.7 mmol) in anhydrous dichloromethane (100 ml), was treated with palladium(II) acetate (263 mg, 1.17 mmol), iodine (2.97 g, 11.7 mmol) and (diacetoxyiodo)benzene (3.77 g, 11.7 mmol); and the reaction was heated in a sealed tube at 65° C. for 16 h. After cooling to ambient temperature, further portions of iodine (2.97 g, 11.7 mmol) and (diacetoxyiodo)benzene (3.77 g, 11.7 mmol) were added; and the reactions was heated at 65° C. for a further 23.5 h. Solvents were evaporated in vacuo, and the crude mixture was purified by silica gel chromatography, eluting with 0 to 3% ethyl acetate in hexanes to give 2-[1-iodo-2-[iodomethyl]decan-2-yl]-4,4-dimethyl-4,5-dihydrooxazole (3.25 g, 55%), as an orange oil. ¹H NMR (400 MHz, CDCl₃): δ 3.95 (s, 2H), 3.58 & 3.47 (ABq, J=9.8 Hz, 4H), 1.66-1.70 (m, 2H), 1.30 (s, 6H), 1.24-1.28 (m, 10H), 1.13-1.22 (m, 2H), 0.87 (t, J=6.9 Hz, 3H).

Step 5: A solution of 2-[1-iodo-2-[iodomethyl]decan-2-yl]-4,4-dimethyl-4,5-dihydrooxazole (3.25 g, 6.43 mmol) in toluene (100 ml), was treated with dibenzoyl peroxide (3.11 g, 12.7 mmol); and the reaction was heated in a sealed tube at 110° C. for 23.5 h. After cooling to ambient temperature, the reaction mixture was diluted with dichloromethane, then washed with saturated aqueous sodium bicarbonate; dried over magnesium sulfate; filtered and evaporated in vacuo, to give the crude product. Purification by silica gel chromatography, eluting with 0 to 5% ethyl acetate in hexanes gave 4,4-dimethyl-2-[1-octylcyclopropyl]-4,5-dihydrooxazole (523 mg, 32%), as a pale yellow oil. ¹H NMR (400 MHz, CDCl₃): δ 3.83 (s, 2H), 1.52-1.56 (m, 2H), 1.36-1.43 (m, 2H), 1.23 (s, 6H), 1.20-1.31 (m, 10H), 1.03 (dd, J=6.6, 4.1 Hz, 2H), 0.88 (t, J=6.9 Hz, 3H), 0.60 (dd, J=6.7, 4.1 Hz, 2H).

Step 6: A solution of 4,4-dimethyl-2-[1-octylcyclopropyl]-4,5-dihydrooxazole (300 mg, 1.19 mmol) in 1,4-dioxane (3 ml), was treated with 4M aqueous sulfuric acid (3 ml); and the reaction was heated in a sealed tube at 100° C. overnight. After cooling to ambient temperature, the reaction mixture was quenched with 2M aqueous sodium hydroxide, and concentrated in vacuo to remove organic solvent. The remaining aqueous phase was washed twice with diethyl ether; acidified with 1M aqueous hydrochloric acid; and extracted twice with dichloromethane. The combined organic extracts were dried over magnesium sulfate; filtered and evaporated in vacuo, to give the crude product. Purification by silica gel chromatography, eluting with 5 to 20% ethyl acetate in hexanes gave 1-octylcyclopropanecarboxylic acid (87 mg, 37%), as a colorless oil. ¹H NMR (400 MHz, CDCl₃): δ 12.16 (br s, 1H), 1.39-1.52 (m, 4H), 1.20-1.32 (m, 10H), 0.87 (t, J=6.9 Hz, 3H), 0.74 (dd, J=7.0, 4.1 Hz, 2H); ¹³C NMR (100 MHz, CDCl₃) δ 182.77, 33.81, 32.12, 30.07, 29.77, 29.54, 27.79, 23.60, 22.90, 16.73, 14.34.

Step 7: 1-Octylcycopropanecarboxylic acid (87 mg, 0.44 mmol) was treated with a solution of sodium bicarbonate (37 mg, 0.44 mmol) in water (0.5 ml), and the mixture was sonicated at 40° C. until a clear, homogeneous solution was obtained. The solution was filtered and lyophilized to give sodium 1-octylcyclopropanecarboxylate (89 mg, 92%) as an off-white solid.

¹H NMR (400 MHz, CD₃OD): δ 1.42-1.52 (m, 4H), 1.22-1.34 (m, 10H), 0.98 (dd, J=6.2, 3.5 Hz, 2H), 0.89 (t, J=6.9 Hz, 3H), 0.44 (dd, J=6.2, 3.6 Hz, 2H); ¹³C NMR (100 MHz, CD₃OD) δ 182.47, 35.60, 31.92, 30.03, 29.73, 29.36, 28.00, 24.94, 22.57, 13.61, 13.27; LRMS (ESI positive): m/z 199.2 (100%, MH⁺ for parent acid); HPLC: 1.2 min (HPLC System: solid phase: Luna C18 75×4.6 mm 5 micron; liquid phase: A=0.01% aqueous trifluoroacetic acid; B=0.01% trifluoroacetic acid in acetonitrile; gradient=80-99% B in A over 5 min).

### Compound VI: Synthesis of sodium 2-(1-heptylcyclopropyl)acetate

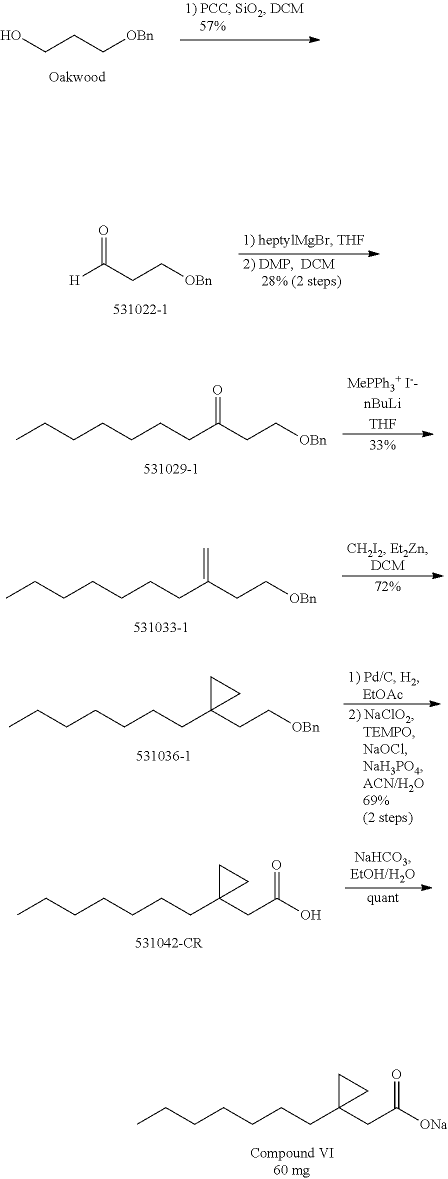

Step 1: 3-(benzyloxy)propanal. A solution of ((3-methylenedecyloxy)methyl)benzene (2.5 g) in dichloromethane (20 ml) at 0° C., was treated portionwise with Dess-Martin Periodinane (8.3 g), then stirred at 0° C. for 30 min. Solvent was evaporated in vacuo, and the crude residue purified by silica gel chromatography, eluting with 0 to 20% ethyl acetate in hexanes, to give 3-(benzyloxy)propanal (1.30 g, 53%).

Step 2: 1-(benzyloxy)decan-3-ol. A solution of 3-(benzyloxy)propanal (1.3 g) in tetrahydrofuran (25 ml) at −78° C. was treated dropwise with a commercial solution of heptylmagnesium bromide in tetrahydrofuran (1.6 M, 8.7 ml). The reaction was stirred at −78° C. for 30 min, then allowed to warm slowly to −20° C. over 60 min. The reaction mixture was quenched by addition of 0.1 M aqueous hydrochloric acid; then extracted with ethyl acetate. The organic extract was dried over sodium sulfate and evaporated in vacuo to give the crude product. Purification by silica gel chromatography, eluting with 0 to 40% ethyl acetate in hexanes, gave partially purified 1-(benzyloxy)decan-3-ol (1.0 g).

Step 3: 1-(benzyloxy)decan-3-one. 1-(benzyloxy)decan-3-ol (1.0 g) is converted to 1-(benzyloxy)decan-3-one in a manner similar to Step 1 of this example to give the desired product (0.56 g, 28% over 2 steps).

Step 4: 3-(benzyloxy)propan-1-ol. A suspension of methyltriphenyl-phosphonium iodide (1.25 g) in tetrahydrofuran (8 ml) at −78° C., was treated with a commercial solution of n-butyllithium in hexanes (2.5 M, 0.94 ml), and the reaction was stirred at −78° C. for 10 min. A solution of 1-(benzyloxy)decan-3-one (0.56 g) in tetrahydrofuran (3 ml) was then added, and the reaction was warmed to 0° C. The reaction was allowed to warm slowly from 0° C. to ambient temperature; and was then quenched by addition of 0.1 M aqueous hydrochloric acid; and extracted with diethyl ether. The organic extract was dried over sodium sulfate and evaporated in vacuo to give the crude product. Purification by silica gel chromatography, eluting with 0 to 5% ethyl acetate in hexanes, gave the desired product (0.38 g, 33%).

Step 5: ((2-(1-heptylcyclopropyl)ethoxy)methyl)benzene. A solution of diiodomethane (0.22 ml) in dichloromethane (5 ml) at 0° C., was treated dropwise with a commercial solution of diethylzinc (1.0 M, 1.38 ml). The reaction was then warmed to ambient temperature; stirred at ambient temperature for 20 min; then re-cooled to 0° C. A solution of ((3-methylenedecyloxy)methyl)benzene (0.18 g) in dichloromethane (2 ml) was added dropwise, and the reaction was warmed to ambient temperature, then stirred at ambient temperature overnight The reaction was quenched by addition of water, then extracted with dichloromethane. The organic extract was dried over sodium sulfate and evaporated in vacuo to give the crude product. Purification by silica gel chromatography, eluting with 0 to 10% ethyl acetate in hexanes, gave the desired product (0.14 g, 72%).

Step 6: 2-(1-heptylcyclopropyl)ethanol. ((2-(1-heptylcyclopropyl)ethoxy)methyl)benzene (0.14 g) is converted to 2-(1-heptylcyclopropyl)ethanol in a manner similar to previous examples (see, e.g., Compound I, Step 5) to give 73 mg of desired product.

Step 7: 2-(1-heptylcyclopropyl)acetic acid. 2-(1-heptylcyclopropyl)ethanol (73 mg) is converted to 2-(1-heptylcyclopropyl)acetic acid in a manner similar to previous examples (see, e.g., Compound I, Step 6) to give 68 mg of desired product.

Step 8: Sodium 2-(1-heptylcyclopropyl)acetate. 2-(1-heptylcyclopropyl)acetic acid (68 mg) is converted to sodium 2-(1-heptylcyclopropyl)acetate in a manner similar to previous examples (see, e.g., Compound I, Step 7) to give 60 mg of the final product. ¹H NMR (400 MHz, Methanol-d4) δ 2.14 (s, 2H), 1.51-1.13 (m, 13H), 0.98-0.79 (m, 3H), 0.52-0.37 (m, 2H), 0.31-0.13 (m, 2H). ¹³C NMR (101 MHz, Methanol-d4) δ 180.06, 44.12, 37.27, 31.70, 29.76, 29.16, 26.44, 22.33, 17.33, 13.02, 11.12. Appearance: white solid. Melting point 158-161° C.

### Compound VII: Synthesis of sodium 2-(1-heptylcyclobutyl)acetate

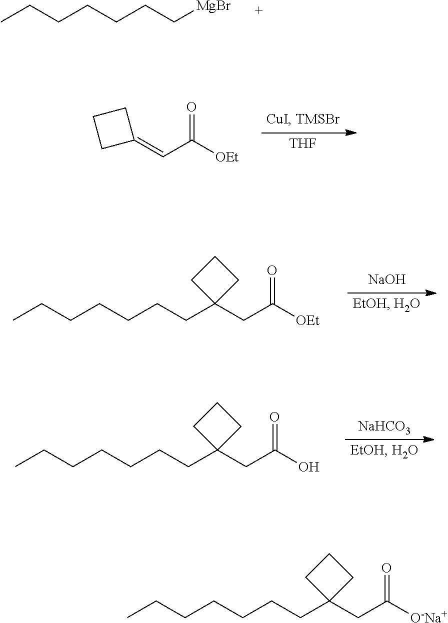

Step 1: ethyl 2-(1-heptylcyclobutyl)acetate. To a solution of ethyl 2-cyclobutylideneacetate (0.2 mL) in THF (8 mL) at 0° C. were added CuI (0.33 g, 1.1 eq.) and TMSBr (0.81 mL, 4 eq.). Reaction was stirred at 0° C. for 40 min., then heptylmagnesium bromide 1M/THF (1.6 mL, 1 eq.) was added dropwise. Reaction was stirred at 0° C. for 4 hours. Reaction was filtered and poured in aqueous saturated NH₄Cl, then MTBE was added. Organic phase was separated, dried over Na₂SO₄, filtered and concentrated. Residue was purified on silica gel (0-3% EA/hexanes) to afford desired ester (49 mg, 13%) as a colorless oil.

Step 2: 2-(1-heptylcyclobutyl)acetic acid. To a solution of ethyl 2-(1-heptylcyclobutyl)acetate (77 mg) in EtOH (2.8 mL) were added H₂O (0.7 mL) and NaOH (64 mg, 5 eq.). Reaction was stirred at reflux for 2 hours. Once at rt, reaction was acidified with 1N HCl until pH 2 was reached. MTBE was added and organic phase was separated, washed with brine, dried over Na₂SO₄, filtered and concentrated to afford desired acid (61 mg, 90%) as a pale yellow oil.

Step 3: Sodium 2-(1-heptylcyclobutyl)acetate. This compound was prepared as for Compound I, Step 7, to afford desired salt (66 mg, quant.) as a white wax. ¹H NMR (400 MHz, Methanol-d₄) δ 2.26 (s, 2H), 2.09-1.99 (m, 2H), 1.90-1.70 (m, 4H), 1.59-1.50 (m, 2H), 1.38-1.20 (m, 10H), 0.95-0.85 (m, 3H). ¹³C NMR (101 MHz, Methanol-d₄) δ 179.86, 46.41, 40.45, 39.64, 31.72, 31.20, 30.24, 29.15, 23.99, 22.34, 14.67, 13.03. ESI-MS m/z 213.18 (M+1).

### Compound VIII: Synthesis of sodium trans-4-pentylcyclohexanecarboxylate

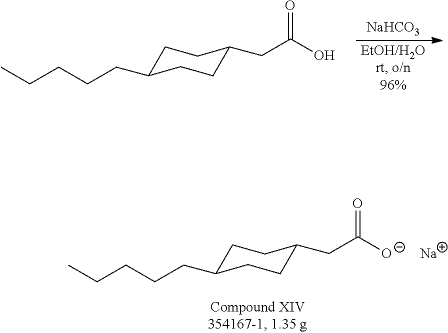

According to the general method for sodium salt formation (as for compound I, step 7), trans-4-pentylcyclohexanecarboxylic acid (1.27 g, 6.40 mmol) was converted to sodium trans-4-pentylcyclohexanecarboxylate (1.26 g, 96%). Mp 302-304° C.; ¹H NMR (400 MHz, CD₃OD): δ 2.07 (ft, J=12.1, 3.5 Hz, 1H), 1.89-1.93 (m, 2H), 1.76-1.80 (m, 2H), 1.14-1.45 (m, 11H), 0.89 (t, J=7.0 Hz, 3H); ¹¹C NMR (100 MHz, CD₃OD) 183.50, 46.84, 37.43, 32.88, 32.20, 30.07, 26.52, 22.58, 13.27; LRMS (ESI positive): m/z 83.0 (100%, unidentified [only m/z]); HPLC: 3.2 min (UPLC System: Mobile phase A=0.01% aqueous TFA; mobile phase B=0.01% TFA in MeCN; solid phase=Luna C18 5 μm; gradient=50-99% B in A over 5 min).

### Compound IX: Synthesis of sodium 3-(4-butylcyclohexyl)propanoate

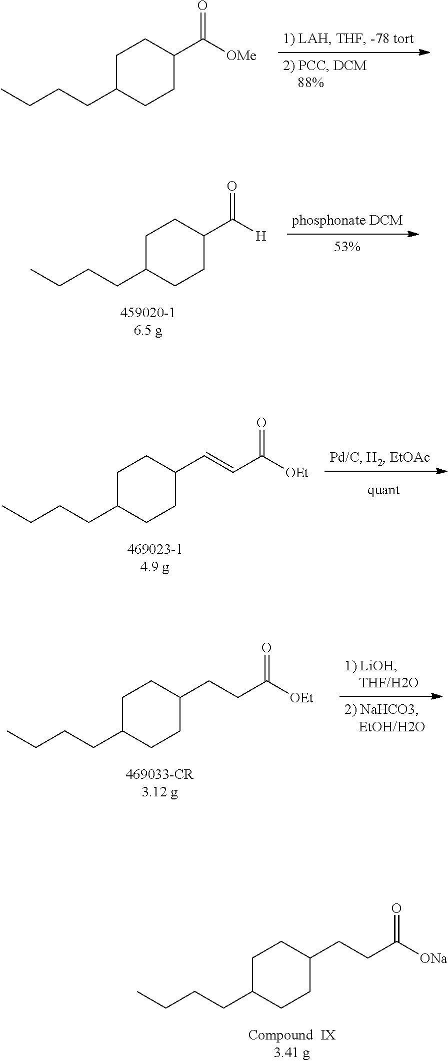

Step 1: (4-butylcyclohexyl)methanol. Methyl 4-butylcyclohexanecarboxylate (15.3 g) is converted to (4-butylcyclohexyl)methanol in a manner similar to previous examples to give 13.1 g of desired product.

Step 2: 4-butylcyclohexanecarbaldehyde. (4-butylcyclohexyl)methanol (7.5 g) is converted to 4-butylcyclohexanecarbaldehyde in a manner similar to previous examples to give 6.5 g of desired product.

Step 3: (E)-ethyl 3-(4-butylcyclohexyl)acrylate. 4-butylcyclohexanecarbaldehyde (6.5 g) was converted to (E)-ethyl 3-(4-butylcyclohexyl)acrylate in a manner similar to previous examples to give 4.9 g of desired product.

Step 4: ethyl 3-(4-butylcyclohexyl)propanoate. (E)-ethyl 3-(4-butylcyclohexyl)acrylate (4.9 g) was converted to ethyl 3-(4-butylcyclohexyl)propanoate in a manner similar to previous examples to give 3.5 g of desired product.

Step 5: 3-(4-butylcyclohexyl)propanoic acid. Ethyl 3-(4-butylcyclohexyl)propanoate (3.5 g) was converted to 3-(4-butylcyclohexyl)propanoic acid in a manner similar to previous examples (see, e.g., Compound IV, Step 2) to give 3.12 g of desired product.

Step 6: Sodium 3-(4-butylcyclohexyl)propanoate. 3-(4-butylcyclohexyl)propanoic acid (3.12 g) was converted to sodium 3-(4-butylcyclohexyl)propanoate in a manner similar to previous examples (see, e.g., Compound I, Step 7) to give 3.41 g of desired product. ¹H NMR (400 MHz, Methanol-d4) δ 2.23-2.08 (m, 2H), 1.84-1.67 (m, 2H), 1.49 (ddd, J=9.9, 7.8, 6.6 Hz, 1H), 1.28 (dt, J=6.8, 3.7 Hz, 2H), 1.23-1.10 (m, 2H), 0.89 (td, J=7.4, 2.4 Hz, 4H). ¹³C NMR (101 MHz, Methanol-d4) δ 182.02, 37.87, 37.73, 36.99, 35.48, 33.97, 33.10, 32.91, 28.99, 22.67, 13.07. Appearance: white solid. Melting point: 292-295° C.

### Compound X: Synthesis of sodium 2-(3-pentylcyclohexyl)acetate

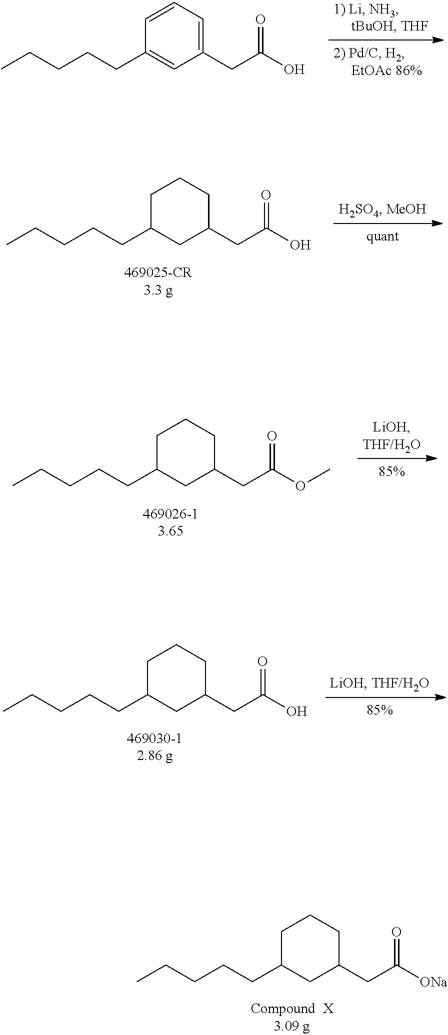

Step 1: 2-(5-pentylcyclohexa-1,4-dienyl)acetic acid. 2-(3-pentylphenyl)acetic acid (5.0 g) was converted to 2-(5-pentylcyclohexa-1,4-dienyl)acetic acid in a manner similar to previous examples to give 3.5 g of desired product.

Step 2: 2-(3-pentylcyclohexyl)acetic acid. 2-(5-pentylcyclohexa-1,4-dienyl)acetic acid was converted to 2-(3-pentylcyclohexyl)acetic acid in a manner similar to previous examples to give 3.3 g of desired product.

Step 3: Methyl 2-(3-pentylcyclohexyl)acetate. 2-(3-pentylcyclohexyl)acetic acid (3.3 g) was converted to methyl 2-(3-pentylcyclohexyl)acetate in a manner similar to previous examples to give 3.65 g of desire product.

Step 4: 2-(3-pentylcyclohexyl)acetic acid. Methyl 2-(3-pentylcyclohexyl)acetate (3.65 g) was converted to 2-(3-pentylcyclohexyl)acetic acid in a manner similar to previous examples (see, e.g., Compound IV, Step 2) to give 2.86 g of desired product.

Step 5: Sodium 2-(3-pentylcyclohexyl)acetate. 2-(3-pentylcyclohexyl)acetic acid (2.86 g) was converted to sodium 2-(3-pentylcyclohexyl)acetate in a manner similar to previous examples (see, e.g., Compound I, Step 7) to give 3.09 g of the final product. ¹H NMR (400 MHz, Methanol-d4) δ 2.08-1.95 (m, 1H), 1.85-1.63 (m, 4H), 1.36-1.08 (m, 9H), 0.93-0.86 (m, 3H), 0.86-0.69 (m, 1H), 0.56 (q, J=11.7 Hz, 1H). ¹³C NMR (101 MHz, Methanol-d4) δ 180.90, 46.22, 40.13, 37.54, 37.48, 35.75, 33.20, 32.96, 32.00, 26.28, 25.97, 22.36, 13.09. Appearance: white solid. Melting point 195-197° C.

### Compound XI: Synthesis of 2-[1-butylpiperidin-4-yl]acetic acid hydrochloride salt

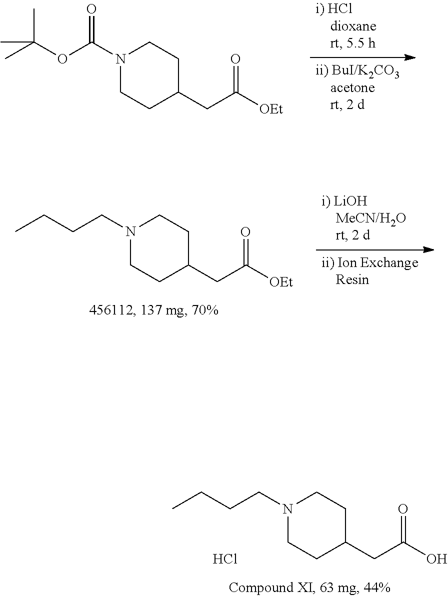 [SMILES: CCOC(=O)CC1CCN(C(=O)OC(C)(C)C)CC1 ; CCCCN1CCC(CC(=O)OCC)CC1 ; CCCCN1CCC(CC(=O)O)CC1 ; Cl]

Step 1: 2-[1-Butylpipeidin-4-yl]acetic Acid, Hydrochloride Salt. A solution of ethyl 2-[1-(tert-butoxycarbonyl)piperidin-4-yl]acetate (246 mg, 0.91 mmol) in dichloromethane (4.7 ml) was cooled to 0° C., under nitrogen. A solution of hydrogen chloride in 1,4-dioxane (4M; 2.5 ml, 12 mmol) was then added, and the reaction was stirred at 0° C., warming slowly to ambient temperature, for 5.5 h. Solvents were evaporated in vacuo to give ethyl 2-[piperidin-4-yl]acetate hydrochloride salt (188 mg, quantitative) as a pale yellow solid. ¹H NMR (400 MHz, CD₃OD): δ 4.12 (q, J=7.0 Hz, 2H), 3.39 (d, J=10.6 Hz, 2H), 2.97-3.07 (m, 2H), 2.30-2.36 (m, 2H), 2.02-2.25 (m, 1H), 1.96 (d, J=12.1 Hz, 2H), 1.49-1.61 (m, 2H), 1.24 (t, J=6.9 Hz, 3H).

Step 2: A solution of ethyl 2-[piperidin-4-yl]acetate hydrochloride salt (188 mg, 0.91 mmol) in acetone (5.2 ml), under nitrogen, was treated with activated 4 Å molecular sieves. Potassium carbonate (268 mg, 1.94 mmol) and 1-iodobutane (0.12 ml, 1.05 mmol) were then added, and the reaction was stirred at 50° C., under nitrogen, for 42 h. Solvents were evaporated in vacuo, and the residue was partitioned between ethyl acetate (20 ml) and 1M aqueous sodium carbonate solution (20 ml). The organic phase was then washed with saturated aqueous sodium chloride solution (20 ml); dried over sodium sulfate; filtered and evaporated in vacuo to give the crude product. Purification by silica gel chromatography, eluting with ethyl acetate then 10% methanol in ethyl acetate gave ethyl 2-[1-butylpiperidin-4-yl]acetate (137 mg, 67%) as a pale yellow oil. ¹H NMR (400 MHz, CD₃OD): δ 4.07 (q, J=7.0 Hz, 2H), 2.85 (d, J=11.7 Hz, 2H), 2.24 (d, J=8.0 Hz, 2H), 2.17 (d, J=7.1 Hz, 2H), 1.86 (t, J=11.7 Hz, 2H), 1.67-1.78 (m, 1H), 1.66 (d, J=14.0 Hz, 2H), 1.38-1.45 (m, 2H), 1.20-1.31 (m, 2H), 1.20 (t, J=7.2 Hz, 3H), 0.86 (t, J=6.8 Hz, 3H).

Step 3: A solution of 2-[1-butylpiperidin-4-yl]acetate (137 mg, 0.60 mmol) in acetonitrile (8 ml) was treated with a solution of lithium hydroxide (76 mg, 3.15 mmol) in water (3.5 ml), and the reaction was stirred at ambient temperature for 48 h. The reaction mixture was loaded onto a Dowex IX2 chloride form ion exchange resin, and the resin was eluted with 10 mM aqueous hydrochloric acid, then 50 mM aqueous hydrochloric acid, to give 2-[1-butylpiperidin-4-yl]acetic acid hydrochloride salt (64 mg, 44%) as a sticky, hygroscopic yellow solid. ¹H NMR (400 MHz, CD₃OD): δ 3.52 (d, J=12.1 Hz, 2H), 3.05 (t, J=8.2 Hz, 2H), 2.95 (t, J=12.1 Hz, 2H), 2.20 (d, J=6.6 Hz, 2H), 1.93-2.07 (m, 3H), 1.67-1.75 (m, 2H), 1.52-1.61 (m, 2H), 1.35-1.44 (m, 2H), 0.98 (t, J=7.5 Hz, 3H); ¹³C NMR (100 MHz, CD₃OD) 176.91, 56.28, 52.09, 41.77, 31.00, 28.80, 25.70, 19.60, 12.53; LRMS (ESI positive): m/z 200.4 (100%, MH⁺); UPLC: 0.8 min (UPLC System: Mobile phase A=0.1% aqueous formic acid; mobile phase B=0.1% formic acid in MeCN; solid phase=HSS C18 1.8 μm; gradient=2-30% B in A over 2.3 min).

### Compound XII: Synthesis of 2-[4-pentylpiperazin-2-yl]acetic acid hydrochloride salt

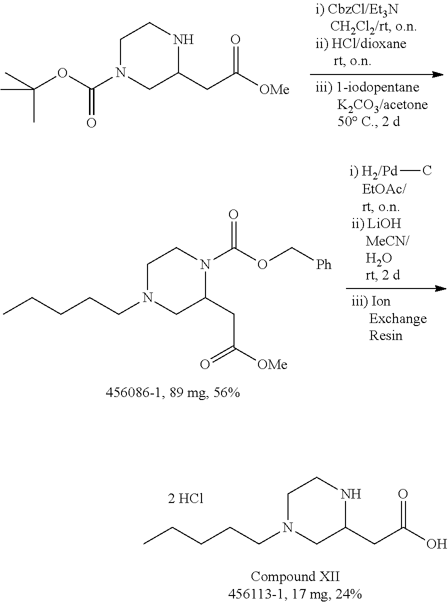

Step 1: 2-[1-Butylpiperidin-4-yl]acetic Acid, Hydrochloride Salt. A solution of methyl 2-[4-(tert-butoxycarbonyl)piperazin-2-yl]acetate (100 mg, 0.39 mmol) in dichloromethane (3.5 ml), under nitrogen, was treated with triethylamine (0.13 ml, 0.93 mmol) and with benzyl chloroformate (140 mg, 0.85 mmol); and the reaction was stirred at ambient temperature, under nitrogen, for 23 h. The solution was washed with 1M aqueous hydrochloric acid (10 ml), with saturated aqueous sodium bicarbonate (10 ml), and with saturated aqueous sodium chloride (10 ml); then dried over sodium sulfate; filtered and evaporated in vacuo to give the crude product. Purification by silica gel chromatography, eluting with a gradient of 0-30% ethyl acetate in hexanes, gave methyl 2-[1-(benzyloxycarbonyl)-4-(tert-butoxycarbonyl)piperazin-2-yl]acetate (133 mg, 87%). ¹H NMR (400 MHz, CDCl₃): δ 7.21-7.40 (m, 5H), 5.10 (s, 2H), 4.53-4.70 (m, 1H), 3.82-4.13 (m, 3H), 3.59 (s, 3H), 2.39-3.08 (m, 5H), 1.41 (s, 9H).

Step 2: Methyl 2-[1-(benzyloxycarbonyl)piperazin-2-yl]acetate hydrochloride salt was prepared as for Compound XI, Step 1 (111 mg, quantitative) as a pale yellow oil. ¹H NMR (400 MHz, CD₃OD): δ 7.29-7.39 (m, 5H), 5.14 (s, 2H), 4.21 (d, J=14.4 Hz, 1H), 3.64-3.75 (m, 2H), 3.59 (s, 3H), 3.46 (d, J=12.1 Hz, 1H), 3.26-3.42 (m, 2H), 3.05-3.12 (m, 1H), 2.82-2.96 (m, 2H).

Step 3: Methyl 2-[1-(benzyloxycarbonyl)-4-pentylpiperazin-2-yl]acetate was prepared as for Compound XI, Step 2 (89 mg, 73%) as a colorless oil. ¹H NMR (400 MHz, CDCl₃): δ 7.24-7.29 (m, 5H), 5.12 (s, 1H), 4.56-4.62 (m, 1H), 3.84-4.00 (m, 1H), 3.59 (s, 3H), 3.04-3.16 (m, 1H), 2.87 (dd, J=14.9, 8.1 Hz, 1H), 2.70-2.85 (m, 1H), 2.63 (dd, J=14.9, 6.3 Hz, 1H), 2.19-2.33 (m, 2H), 2.06-2.11 (m, 1H), 1.93-2.00 (m, 1H), 1.37-1.45 (m, 2H), 1.23-1.32 (m, 4H), 0.88 (t, J=6.9 Hz, 3H).

Step 4: A solution of methyl 2-[1-(benzyloxycarbonyl)-4-pentylpiperazin-2-yl]acetate (89 mg, 0.25 mmol) in ethyl acetate (2.5 ml), under nitrogen, was treated with 10% w/w palladium on activated carbon (15 mg). The mixture was then stirred at ambient temperature, under a hydrogen atmosphere, for 17 h. The mixture was filtered through Celite™, and the residue was washed with ethyl acetate. Filtrates were evaporated in vacuo to give methyl 2-[4-pentylpiperazin-2-yl]acetate (53 mg, 99%) as a colorless oil. ¹H NMR (400 MHz, CDCl₃): δ 3.65 (s, 3H), 3.11-3.17 (m, 1H), 2.85-2.96 (m, 2H), 2.73-2.77 (m, 2H), 2.34-2.36 (m, 2H), 2.27 (t, J=7.8 Hz, 2H), 1.94-2.02 (m, 1H), 1.73 (t, J=10.6 Hz, 1H), 1.40-1.48 (m, 2H), 1.19-1.32 (m, 4H), 0.85 (t, J=7.1 Hz, 3H).

Step 5: 2-[4-pentylpiperazin-2-yl]acetic acid hydrochloride salt was prepared as for Compound XI, Step 3 (17 mg, 24%) as a white solid. ¹H NMR (400 MHz, CD₃OD): δ 3.32-3.39 (m, 1H), 3.25 (dt, J=12.5, 2.7 Hz, 1H), 3.06 (td, 12.5, 2.9 Hz, 1H), 2.98 (t, J=14.6 Hz, 2H), 2.34-2.44 (m, 4H), 2.98 (t, J=7.8 Hz, 2H), 2.27 (td, 11.8, 2.7 Hz, 1H), 2.11 (t, J=10.3 Hz, 1H), 1.48-1.55 (m, 2H), 1.26-1.39 (m, 4H), 0.91 (t, J=7.0 Hz, 3H); ¹³C NMR (100 MHz, CD₃OD) 176.06, 57.81, 55.50, 52.75, 50.20, 43.14, 37.62, 29.22, 25.64, 22.16, 12.92; LRMS (ESI positive): m/z 215.4 (100%, MH⁺); UPLC: 0.4 min (UPLC System: Mobile phase A=0.1% aqueous formic acid; mobile phase B=0.1% formic acid in MeCN; solid phase=HSS C18 1.8 μm; gradient=2-30% B in A over 2.3 min).

### Compound XIII: Synthesis of 2-[1-Pentylpiperdin-4-yl]acetic Acid, Hydrochloride Salt

This compound was prepared in the same manner as Compound XI, replacing 1-iodobutane with 1-iodopentane. ¹H NMR (400 MHz, CD₃OD): δ3.47 (d, J=11.4 Hz, 2H), 2.96-3.00 (m, 2H), 2.86 (t, J=12.5 Hz, 2H), 2.13 (d, J=5.8 Hz, 2H), 1.90-2.01 (m, 3H), 1.67-1.76 (m, 2H), 1.48-1.57 (m, 2H), 1.30-1.41 (m, 4H), 0.93 (t, J=7.0 Hz, 3H); ¹³C NMR (100 MHz, CD₃OD) 178.57, 56.54, 52.19, 43.37, 31.54, 29.07, 28.51, 23.50, 21.85, 12.78; LRMS (ESI positive): m/z 214.4 (100%, MH⁺); UPLC: 1.1 min (UPLC System: Mobile phase A=0.1% aqueous formic acid; mobile phase B=0.1% formic acid in MeCN; solid phase=HSS C18 1.8 μm; gradient=2-30% B in A over 2.3 min).

### Compound XIV: Synthesis of sodium (E)-6-cyclohexylhex-2-enoate

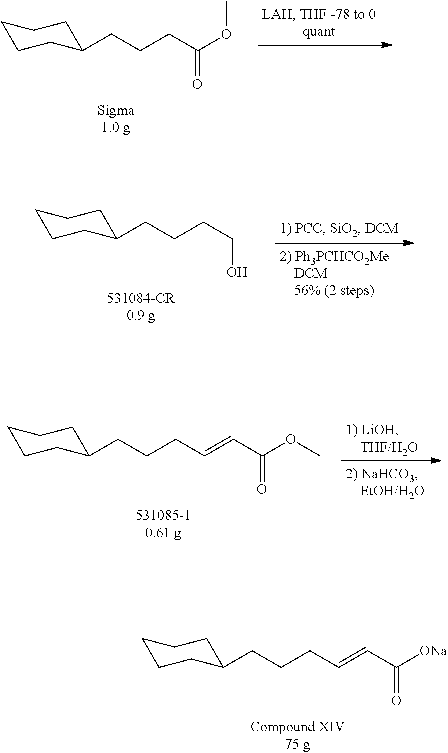

Step 1: 4-cyclohexylbutan-1-ol. Methyl 4-cyclohexylbutanoate (1.0 g) was converted to 4-cyclohexylbutan-1-ol in a manner similar to previous examples (see, e.g., Compound I, Step 2) to give 0.9 g of desired product.

Step 2: 4-cyclohexylbutanal. 4-cyclohexylbutan-1-ol (0.9 g) was converted to 4-cyclohexylbutanal in a manner similar to previous examples to give 0.8 g of desired product.

Step 3: (E)-methyl 6-cyclohexylhex-2-enoate. 4-cyclohexylbutanal (0.80 g) was converted to (E)-methyl 6-cyclohexylhex-2-enoate in a manner similar to previous examples to give 0.61 of desired product.

Step 4: (E)-6-cyclohexylhex-2-enoic acid. (E)-methyl 6-cyclohexylhex-2-enoate (0.15 g) was converted to (E)-6-cyclohexylhex-2-enoic acid in a manner similar to previous examples (see, e.g., Compound I, Step 2) to give 77 mg of desired product.

Step 5: Sodium (E)-6-cyclohexylhex-2-enoate. (E)-6-cyclohexylhex-2-enoic acid (77 mg) was converted to sodium (E)-6-cyclohexylhex-2-enoate in a manner similar to previous examples (see, e.g., Compound I, Step 7) to give 73 mg of the final product. ¹H NMR (400 MHz, Methanol-d4) δ 6.60 (dt, J=15.5, 7.0 Hz, 1H), 5.80 (dt, J=15.5, 1.5 Hz, 1H), 2.10 (qd, J=7.3, 1.4 Hz, 2H), 1.82-1.57 (m, 6H), 1.44 (p, J=7.5 Hz, 3H), 1.35-1.05 (m, 7H), 0.88 (q, J=10.7, 9.4 Hz, 2H). ¹³C NMR (101 MHz, Methanol-d4) δ 174.53, 142.69, 127.60, 37.45, 36.78, 33.11, 31.83, 26.39, 26.09, 25.63. Appearance: white solid.

### Compound X: Synthesis of sodium 4-pentylbicyclo[2.2.2]octane-1-carboxylate

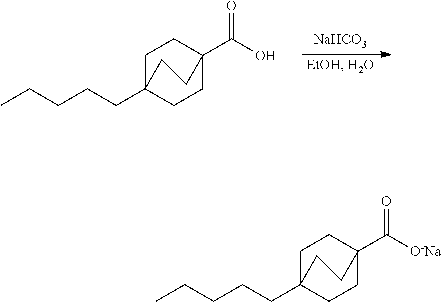

Sodium 4-pentylbicyclo(2.2.2)octane-1-carboxylate was prepared as for Compound I, Step 7 from commercially available 4-pentylbicyclo(2.2.2)octane-1-carboxylic acid (66 mg, quant.) as a white solid. ¹H NMR (400 MHz, Methanol-d₄) δ1.76-1.69 (m, 6H), 1.38-1.27 (m, 8H), 1.23-1.15 (m, 4H), 1.08-1.01 (m, 2H), 0.88 (t, J=7.2 Hz, 3H). ¹³C NMR (101 MHz, Methanol-d₄) δ 186.08, 41.51, 40.00, 32.71, 30.90, 30.05, 29.21, 23.06, 22.31, 13.01. ESI-MS m/z 179.29 (M-COOH). Melting point: >300° C.

### Compounds XVI and XVII: Synthesis of sodium 3-pentylcyclobutanecarboxylate and disodium 3-pentylcyclobutane-1,1-dicarboxylate

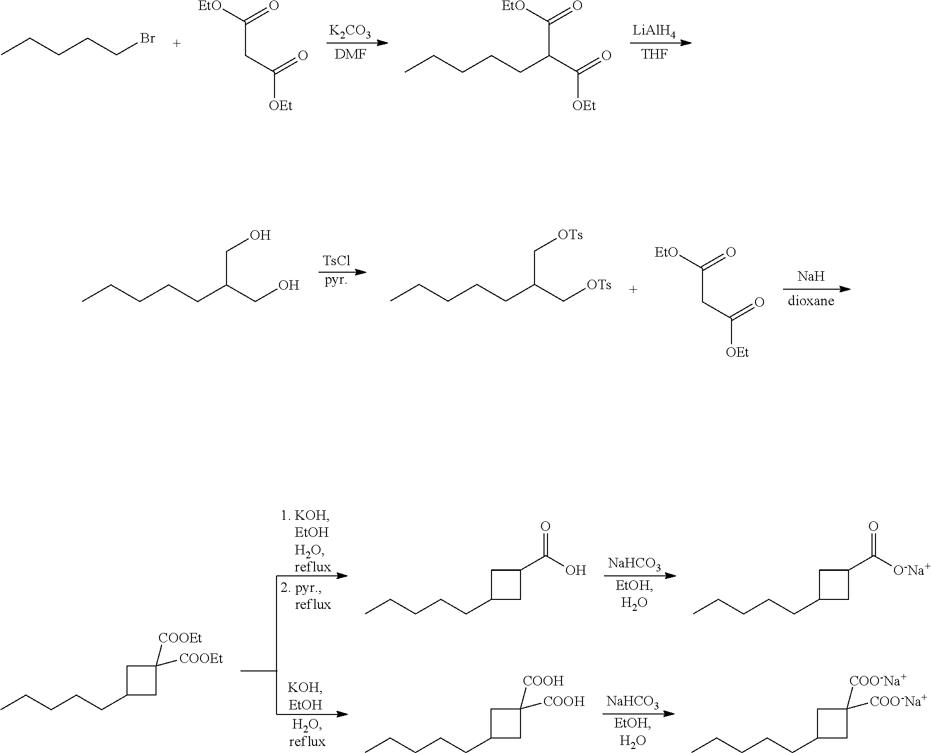

Step 1: diethyl 2-pentylmalonate. To a solution of 1-bromopentane (2.5 mL) in DMF (100 mL) were added diethyl malonate (6.1 mL, 2 eq.) and K₂CO₃ (7 g, 2.5 eq.). Reaction was stirred at rt for 18 hours. Reaction was poured in aq. sat. NH₄Cl and EA was added. Organic phase was separated, dried over Na₂SO₄, filtered and concentrated. Residue was purified on silica gel (0-20% EA/hexanes) to afford desired alkyl malonate (3.95 g, 85%) as a colorless oil (see WO 2006/091790A1).

Step 2: 2-pentylpropane-1,3-dol. To a suspension of LiAlH₄ (1.3 g, 2 eq.) in THF (65 mL) was slowly added a solution of diethyl 2-pentylmalonate (3.95 g) in THF (10 mL). Reaction was stirred at reflux for 3 hours. Once at rt, another amount of LiAlH₄ (1.3 g, 2 eq.) was added and the reaction was stirred at rt for 18 hours. Reaction was cooled down to 0° C. and H₂O was slowly added followed by 1N HCl. MTBE was added and org. phase was separated. Aq. phase was extracted with MTBE. Combined org. phases were washed with brine, dried over Na₂SO₄, filtered and concentrated to afford desired diol (2.48 g, 99%) as a pale yellow oil (see, Macromolecules, 41(3), 691, 2008).

Step 3: 2-pentylpropane-1,3-diyl bis(4-methylbenzenesulfonate). To a solution of 2-pentylpropane-1,3-diol (2.48 g) in pyridine (50 mL) at 0° C. was added TsCl (8.08 g, 2.5 eq.). Reaction was allowed to warm up to rt over 3 hours. Another amount of TsCl (3.2 g, 1 eq.) was added and the reaction was stirred at rt for 18 hours. Reaction was poured in water and MTBE was added. Org. phase was separated, washed with 1N HCl (3×) and brine, dried over Na₂SO₄, filtered and concentrated. Residue was purified on silica gel (0-30% EA/hexanes) to afford desired bis-tosylate (2.3 g, 30%) as a colorless oil (see, Macromolecules, 41(3), 691, 2008).

Step 4: diethyl 3-pentylcyclobutane-1,1-dicarboxylate. To a solution of 2-pentylpropane-1,3-diyl bis(4-methylbenzenesulfonate) (2.3 g) in dioxane (22 mL) was added diethyl malonate (0.86 mL, 1.1 eq.). Reaction was stirred at reflux and NaH 60% w/w (0.41 g, 2 eq.) was added by small portions over 1 hour. Reaction was stirred at reflux for 18 hours. Once at rt, reaction was poured in water and MTBE was added. Organic phase was separated, washed with brine, dried over Na₂SO₄, filtered and concentrated. Residue was purified on silica gel (0-10% EA/hexanes) to afford desired cyclobutane (811 mg, 58%) as a pale yellow oil (see European Journal of Organic Chemistry 17: 3584-3591, 2014).

Step 5A: 3-pentylcyclobutanecarboxylic acid, cis/trans mixture. To a solution of diethyl 3-pentylcyclobutane-1,1-dicarboxylate (150 mg) in EtOH (1 mL) were added H₂O (90 μL) and KOH (157 mg, 5 eq.). Reaction was stirred at reflux for 3 hours. Once at rt, reaction was concentrated. Residue was dissolved in 1N HCl and MTBE. Organic phase was separated, washed with brine, dried over Na₂SO₄, filtered and concentrated. Residue was dissolved in pyridine (2.8 mL) and resulting mixture was stirred at reflux for 18 hours. Once at rt, reaction was poured in 1N HCl and MTBE was added. Organic phase was separated, washed with 1N HCl (2×) and brine, dried over Na₂SO₄, filtered and concentrated to afford desired mixture of cis/trans acid (88 mg, 93%) as a pale yellow oil (see WO 2009/114512A1).

Step 6A: Sodium 3-pentylcyclobutanecarboxylate, cis/rans mixture. To a solution of 3-pentylcyclobutanecarboxylic acid, cis/trans mixture (88 mg) in EtOH (3.9 mL) were added H₂Oₙₐₙₒ (1.3 mL) and NaHCO₃ (43 mg, 1 eq.). Reaction was stirred at rt for 18 hours. Reaction was concentrated and dissolved in H₂Oₙₐₙₒ. Solution was filtered through 0.2 μm PES filter and filtrate was lyophilized to afford desired salt (99 mg, quant.) as an off-white solid. ¹H NMR (400 MHz, Methanol-d₄) δ 2.99-2.69 (m, 1H), 2.36-1.98 (m, 3H), 1.83-1.69 (m, 2H), 1.43 (q, J=7.5 Hz, 1H), 1.39-1.15 (m, 7H), 0.88 (td, J=7.0, 2.9 Hz, 3H). ¹³C NMR (101 MHz, Methanol-d₄) δ 184.09, 183.36, 37.70, 37.47, 36.78, 36.37, 32.34, 31.67, 31.64, 31.53, 31.25, 31.01, 26.74, 26.49, 22.34, 22.33, 12.99, 12.98. ESI-MS m/z 125.20 (M-COOH). MP: 244-254° C.

Step 5B: 3-pentylcyclobutane-1,1-dicarboxylic acid. To a solution of diethyl 3-pentylcyclobutane-1,1-dicarboxylate (150 mg) in EtOH (1 mL) were added H₂O (90 μL) and KOH (157 mg, 5 eq.). Reaction was stirred at reflux for 5 hours. Once at rt, reaction was concentrated. Residue was dissolved in 1N HCl and MTBE. Org. phase was separated, washed with brine, dried over Na₂SO₄, filtered and concentrated to afford desired diacid (118 mg, 99%) as a white solid (see WO 2009/114512A1).

Step 6B: disodium 3-pentylcyclobutane-1,1-dicarboxylate. The compound was prepared in a similar manner to Compound I, step 7 to afford the desired salt (135 mg, 99%) as a white solid. ¹H NMR (400 MHz, Deuterium Oxide) δ 2.27 (ddd, J=10.3, 8.4, 2.4 Hz, 2H), 2.08-1.87 (m, 1H), 1.85-1.73 (m, 2H), 1.31-0.97 (m, 8H), 0.69 (t, J=6.9 Hz, 3H). ¹³C NMR (101 MHz, Deuterium Oxide) δ 182.86, 182.55, 54.43, 36.56, 36.48, 31.09, 28.67, 25.95, 21.99, 13.30. ESI-MS m/z 214.98 (M+1). MP: >300° C.

### Compounds XVIII and XIX: Synthesis of sodium 2-(3-pentylcyclobutyl)acetate and 2-(3-pentylcyclobutylidene)acetate

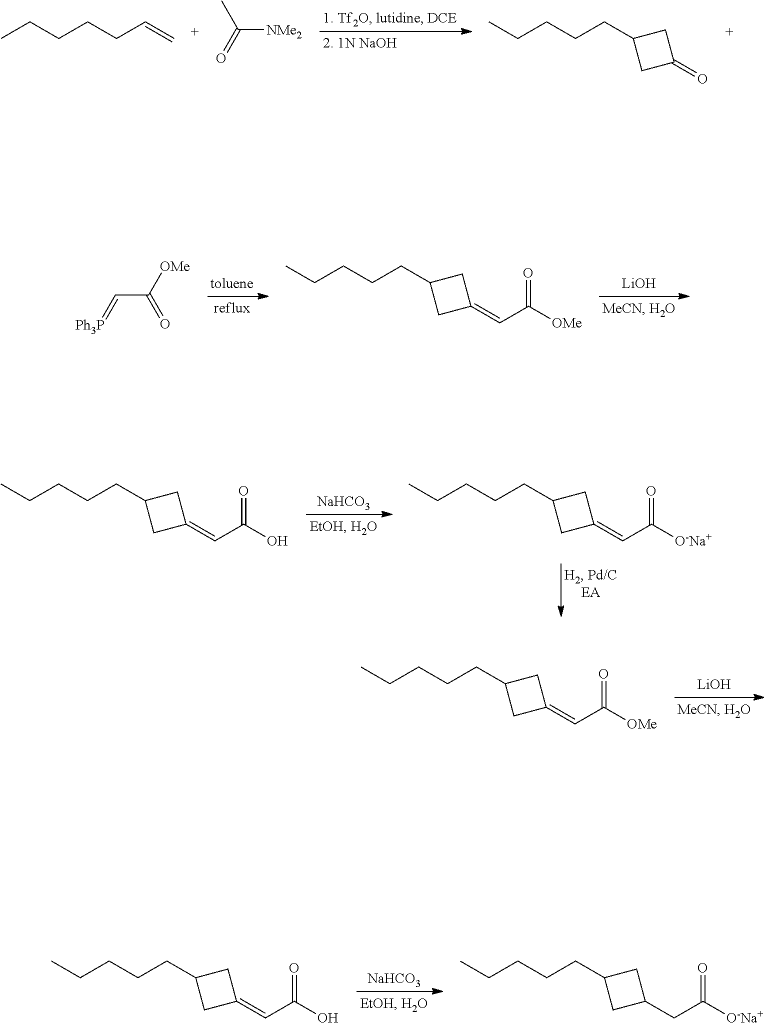

Step 1: 3-pentylcyclobutanone. To a solution of N,N-dimethylacetamide (330 μL) in DCE (10 mL) at −15° C. was added dropwise Tf₂O (0.7 mL, 1.2 eq.). And then, a solution of hept-1-ene (2 mL, 4 eq.) and lutidine (0.5 mL, 1.2 eq.) in DCE (5 mL) was added dropwise at −15° C. Reaction was stirred at reflux for 18 hours. Once at rt, reaction was concentrated. 1N NaOH was added and reaction was stirred at 60° C. for 50 min. Once at rt, reaction was poured in aq. sat. NH₄Cl and hexanes was added. Organic phase was separated, washed with aq. sat. NH₄Cl (3×), dried over Na₂SO₄, filtered and concentrated. Residue was purified on silica gel (0-4% EA/hexanes) to afford desired cyclobutanone (296 mg, 59%) as a colorless oil (see Organic Syntheses, Coll. Vol. 8, p. 306 (1993); Vol. 69, p. 199 (1990)).

Step 2: Methyl 2-(3-pentylcyclobutylidene)acetate, cis/trans mixture. To a solution of 3-pentylcyclobutanone (295 mg) in toluene (20 mL) was added methyl (triphenylphosphoranylidene)acetate (914 mg, 1.3 eq.). Reaction was stirred at reflux for 18 hours. Once at rt, reaction was concentrated and residue was purified on silica gel (0-4% EA/hexanes) to afford desired alkene cis/trans mixture (252 mg, 61%) as a colorless oil (see Yvonne Lear, U. Ottawa, thesis, 1997, doi: 10.20381/ruor-13853).

Step 3: 2-(3-pentylcyclobutylidene)acetic acid, cis/trans mixture. This compound was prepared as for Compound IV, step 2 (59 mg, 51%) as a colorless oil.

Step 4: Sodium 2-(3-pentylcyclobutylidene)acetate (Compound XIX), cis/trans mixture. This compound was prepared as for Compound I, step 7 (63 mg, 99%) as a white solid. ¹H NMR (400 MHz, Methanol-d₄) δ 5.60-5.53 (m, 1H), 3.26-3.11 (m, 1H), 2.88-2.73 (m, 1H), 2.65-2.54 (m, 1H), 2.36-2.20 (m, 2H), 1.52-1.40 (m, 2H), 1.40-1.21 (m, 6H), 0.97-0.83 (m, 3H). ¹³C NMR (101 MHz, Methanol-d₄) δ 174.84, 154.97, 118.98, 38.20, 37.06, 36.41, 31.59, 31.27, 26.90, 22.32, 12.98. ESI-MS m/z 183.18 (M+1). MP: 264-267° C.

Step 1B: Methyl 2-(3-pentylcyclobutyl)acetate, cis/rans mixture. To a N₂ bubbled solution of methyl 2-(3-pentylcyclobutylidene)acetate, cis/trans mixture (125 mg) in ethyl acetate (7 mL) was added Pd/C 10% w/w (68 mg, 0.1 eq.). N₂ was removed and H₂ was bubbled in the reaction for 5 min. And then, reaction was stirred under H₂ atmosphere for 18 hours. H₂ was removed and N₂ was bubbled. Celite™ was added and reaction was filtered on Celite™. Filtrate was concentrated to afford desired mixture of ester diastereoisomers (110 mg, 87%) as a pale yellow oil.

Step 2B: 2-(3-pentylcyclobutyl)acetic acid, cis/trans mixture. This compound was prepared as for Compound IV, Step 2 (100 mg, 99.5%) as a pale yellow oil.

Step 3B: Sodium 2-(3-pentylcyclobutyl)acetate (Compound XVIII), cis/trans mixture. This compound was prepared as for Compound I, Step 7 (109 mg, 98%) as a white solid. ¹H NMR (400 MHz, Methanol-d₄) δ 2.67-2.38 (m, 1H), 2.36-2.15 (m, 4H), 2.12-1.70 (m, 2H), 1.47-1.13 (m, 9H), 0.93-0.84 (m, 3H). ¹³C NMR (101 MHz, Methanol-d₄) δ 180.75, 180.53, 45.69, 44.77, 37.28, 36.44, 34.76, 32.62, 32.01, 31.68, 31.65, 31.27, 29.57, 29.27, 26.81, 26.64, 22.33, 12.98. ESI-MS m/z 185.28 (M+1).

### Compounds XX and XXI: Synthesis of sodium 3-hexylidenecyclobutanecarboxylate and 3-hexylcyclobutanecarboxylate

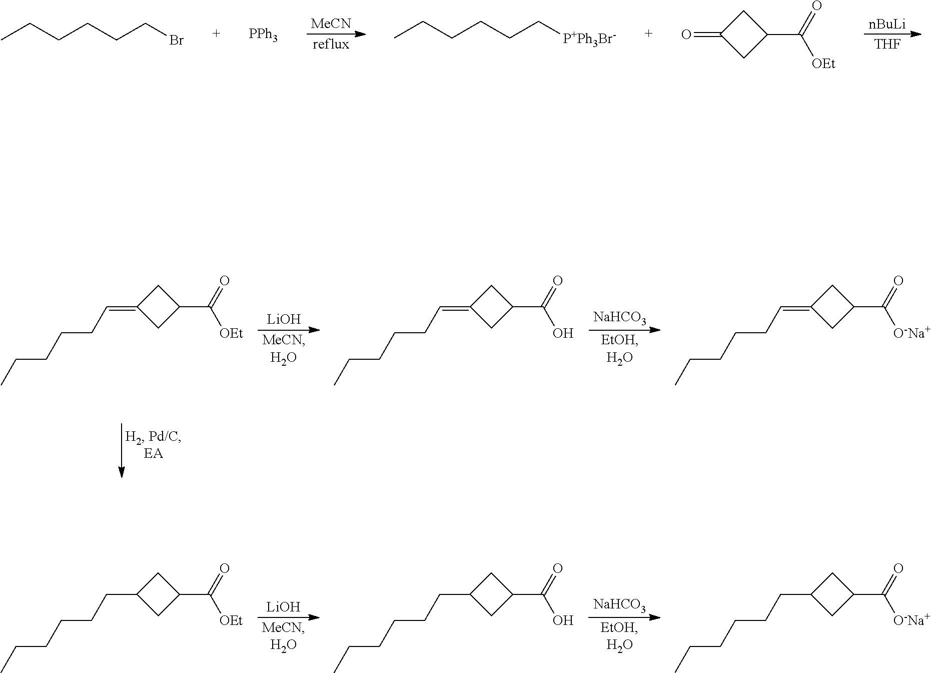

Step 1: Hexyltriphenylphosphonium bromide. To a solution of 1-bromohexane (10.7 mL, 2 eq.) in MeCN (190 mL) was added PPh₃ (10 g). Reaction was stirred at reflux for 66 hours. Once at rt, reaction mixture was washed with hexanes (3×) and concentrated to afford desire phosphonium salt (16.2 g, 99%) as an off-white solid (see J. Nat. Prod., 67(8), 1277, 2004).

Step 2: Ethyl 3-hexylidenecyclobutanecarboxylate, cis/trans mixture. To a suspension of hexyltriphenylphosphonium bromide (4.2 g, 1.2 eq.) in THF (10 mL) at −78° C. was added dropwise nBuLi 2.5M/hex. Reaction was allowed to warm up to 0° C. for a stirring of 20 min. Reaction was cooled down to −78° C. and a solution of ethyl 3-oxocyclobutanecarboxylate (1 mL) in THF (5 mL) was added dropwise. Reaction was warmed up to rt and stirred at rt for 18 hours. Reaction was poured in H₂O and MTBE was added. Org. phase was separated, washed with H₂O and brine, dried over Na₂SO₄, filtered and concentrated. Residue was purified on silica gel (0-4% EA/hexanes) to afford desired alkene cis/trans mixture (168 mg, 10%) as a colorless oil. (J. Med. Chem., 49(1), 80, 2006).

Step 3: 3-hexylidenecyclobutanecarboxylic acid, cis/trans mixture. This compound was prepared as for Compound IV, Step 2 (63 mg, 88%) as a colorless oil.

Step 4: Sodium 3-hexylidenecyclobutanecarboxylate (compound X), cis/trans mixture. This compound was prepared as for Compound I, Step 7 (66 mg, 96%) as a white solid. ¹H NMR (400 MHz, Methanol-d₄) δ 5.05 (tp, J=7.0, 2.2 Hz, 1H), 2.98-2.68 (m, 5H), 1.87 (q, J=7.2, 6.6 Hz, 2H), 1.38-1.20 (m, 6H), 0.96-0.84 (m, 3H). ¹³C NMR (101 MHz, Methanol-d₄) δ 182.88, 135.39, 120.33, 36.32, 34.75, 33.13, 31.17, 29.10, 27.53, 22.21, 13.01. ESI-MS m/z 183.28 (M+1).

Step 1B: Ethyl 3-hexylcyclobutanecarboxylate, cis/trans mixture. To a N₂ bubbled solution of ethyl 3-hexylidenecyclobutanecarboxylate, cis/trans mixture (83 mg) in ethyl acetate (5 mL) was added Pd/C 10% w/w (42 mg, 0.1 eq.). N₂ was removed and H₂ was bubbled in the reaction for 5 min. And then, reaction was stirred under H₂ atmosphere for 18 hours. H₂ was removed and N₂ was bubbled. Celite™ was added and reaction was filtered on Celite™. Filtrate was concentrated to afford desired ester cis/trans mixture (83 mg, 99%) as a colorless oil.

Step 2B: 3-hexylcyclobutanecarboxylic acid, cis/trans mixture. This compound was prepared as for Compound IV, Step 2 (64 mg, 91%) as a colorless oil.

Step 3B: Sodium 3-hexylcyclobutanecarboxylate (compound XXI), cis/trans mixture. This compound was prepared as for Compound I, Step 7 (71 mg, quant.) as a white solid. ¹H NMR (400 MHz, Methanol-d₄) δ 2.99-2.70 (m, 1H), 2.37-1.98 (m, 3H), 1.84-1.68 (m, 2H), 1.48-1.14 (m, 10H), 0.94-0.84 (m, 3H). ¹³C NMR (101 MHz, Methanol-d₄) δ 183.37, 37.72, 37.49, 36.81, 36.41, 32.35, 31.67, 31.50, 31.24, 31.00, 29.08, 29.04, 27.03, 26.78, 22.28, 13.00. ESI-MS m/z 138.39 (M-COOH). MP: 247-250° C.

### Compound XXII: Synthesis of sodium 2-(2,2-dimethyl-3-pentylcyclobutyl)acetate

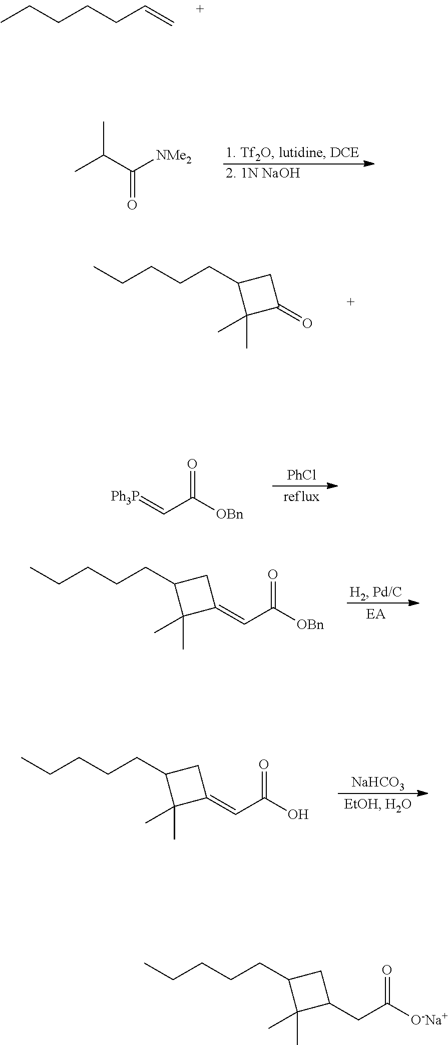

Step 1: 2,2-dimethyl-3-pentylcyclobutanone. To a solution N,N-dimethylisobutyramide (0.46 mL) in DCE (10 mL) at −15° C. was added dropwise Tf₂O (0.7 mL, 1.2 eq.). And then, a solution of hept-1-ene (2 mL, 4 eq.) and lutidine (0.5 mL, 1.2 eq.) in DCE (5 mL) was added dropwise at −15° C. Reaction was stirred at reflux for 18 hours. Once at rt, reaction was concentrated. 1N NaOH was added and reaction was stirred at 60° C. for 1 hour. Once at rt, MTBE was added. Org. phase was separated, washed with brine, dried over Na₂SO₄, filtered and concentrated. Residue was purified on silica gel (0-4% EA/hexanes) to afford desired cyclobutanone (231 mg, 39%) as a pale yellow oil (Organic Syntheses, Coll. Vol. 8, p. 306 (1993); Vol. 69, p. 199 (1990)).

Step 2: (E)-benzyl 2-(2,2-dimethyl-3-pentylcyclobutylidene)acetate. To a solution of 2,2-dimethyl-3-pentylcyclobutanone (230 mg) in chlorobenzene (10 mL) was added benzyl (triphenylphosphoranylidene)acetate (1.12 g, 2 eq.). Reaction was stirred at reflux for 18 hours. Once at rt, another amount of benzyl (triphenylphosphoranylidene)acetate (1.12 g, 2 eq.) was added and the reaction was stirred at reflux for 3 days. Once at rt, reaction was concentrated and residue was purified on silica gel (0-3% EA/hexanes) to afford desired alkene (226 mg, 55%) as a colorless oil (Yvonne Lear, U. Ottawa, thesis, 1997, doi: 10.20381/ruor-13853).

Step 3: 2-(2,2-dimethyl-3-pentylcyclobutyl)acetic acid. To a N₂ bubbled solution of (E)-benzyl 2-(2,2-dimethyl-3-pentylcyclobutylidene)acetate (254 mg) in ethyl acetate (10 mL) was added Pd/C 10% w/w (90 mg, 0.1 eq.). N₂ was removed and H₂ was bubbled in the reaction for 5 min. And then, reaction was stirred under H₂ atmosphere for 18 hours. H₂ was removed and N₂ was bubbled. Celite™ was added and reaction was filtered on Celite™. Filtrate was concentrated to afford desired diastereoisomers mixture (176 mg, 98%) as a colorless oil.

Step 4: Sodium 2-(2,2-dimethyl-3-pentylcyclobutyl)acetate. This compound was prepared as for Compound I, Step 7 (189 mg, 98%) as a white solid. ¹H NMR (400 MHz, Methanol-d₄) δ 2.33-1.97 (m, 4H), 1.87-1.62 (m, 2H), 1.48-1.09 (m, 8H), 1.06-0.85 (m, 9H). ¹³C NMR (101 MHz, Methanol-d₄) δ 181.24, 181.07, 43.13, 41.63, 40.01, 39.72, 39.26, 38.98, 38.68, 38.08, 31.91, 31.89, 30.51, 30.13, 30.01, 29.23, 28.40, 27.27, 23.54, 22.85, 22.34, 22.31, 15.66, 13.00. ESI-MS m/z 213.18 (M+1).

### Compound XXIII: Synthesis of sodium 2-(2-hexylcyclopropyl)acetate

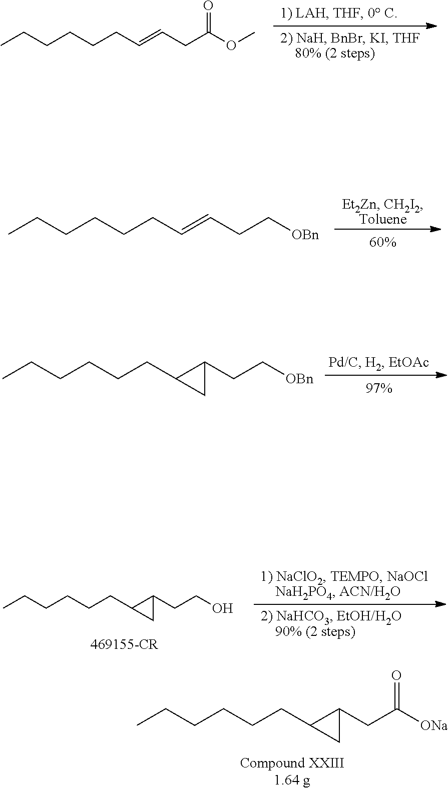

Step 1: (E)-dec-3-en-1-ol. (E)-methyl dec-3-enoate (9.0 g) is converted to (E)-dec-3-en-1-ol in a manner similar to previous examples (see, e.g., Compound I, step 2) to give 7.5 g of desired product.

Step 2: (E)-((dec-3-enyloxy)methyl)benzene. (E)-dec-3-en-1-ol (7.5 g) is converted to (E)-((dec-3-enyloxy)methyl)benzene in a manner similar to previous examples (see, e.g., Compound I, step 3) to give 9.7 g of desired product.

Step 3: ((2-(2-hexylcyclopropyl)ethoxy)methyl)benzene. (E)-((dec-3-enyloxy)methyl)benzene (4.0 g) was converted to ((2-(2-hexylcyclopropyl)ethoxy)methyl)benzene in a manner similar to previous examples (see, e.g., Compound VI, step 4) to give 2.5 g of desired product.

Step 4: 2-(2-hexylcyclopropyl)ethanol. ((2-(2-hexylcyclopropyl)ethoxy)methyl)benzene (2.5 g) was converted to 2-(2-hexylcyclopropyl)ethanol in a manner similar to previous examples (see, e.g., Compound I, step 5) to give 1.57 g of desired product.

Step 5: 2-(2-hexylcyclopropyl)acetic acid. 2-(2-hexylcyclopropyl)ethanol (1.57 g) was converted to 2-(2-hexylcyclopropyl)acetic acid in a manner similar to previous examples (see, e.g., Compound I, step 6) to give 1.50 g of desired product.

Step 6: Sodium 2-(2-hexylcyclopropyl)acetate. 2-(2-hexylcyclopropyl)acetic acid (1.50 g) was converted to sodium 2-(2-hexylcyclopropyl)acetate in a manner similar to previous examples (see, e.g., Compound I, step 7) to give 1.6 g of the final product. ¹H NMR (400 MHz, Methanol-d4) δ 2.13 (dd, J=14.2, 6.7 Hz, 1H), 1.98 (dd, J=14.2, 7.4 Hz, 1H), 1.44-1.11 (m, 11H), 0.89 (t, J=6.9 Hz, 3H), 0.78 (ddt, J=11.8, 7.1, 3.9 Hz, 1H), 0.50 (ddt, J=11.1, 6.9, 3.5 Hz, 1H), 0.27 (dt, J=8.4, 4.6 Hz, 1H), 0.20 (dt, J=9.3, 4.7 Hz, 1H). Appearance: white solid. Melting point: 189-192° C.

### Compound XXIV: Synthesis of sodium 2-(2,3-dihexylcyclopropyl)-2-oxoacetate

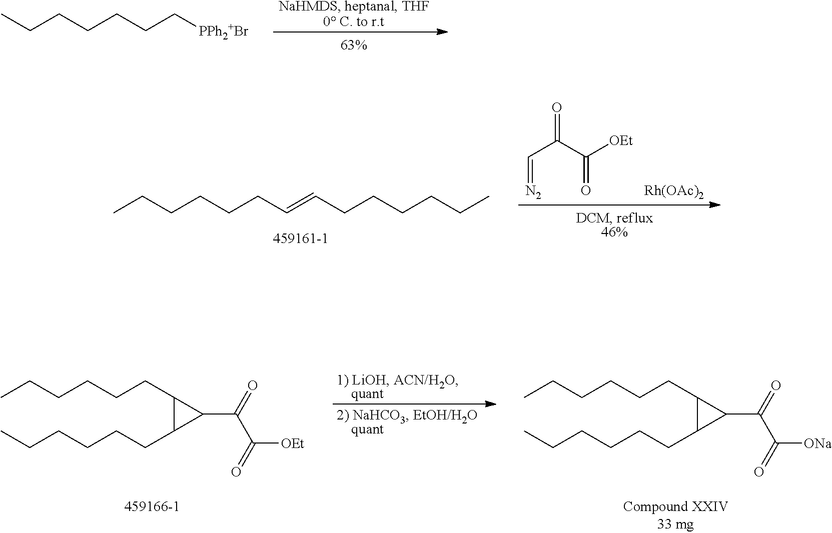

Step 1: (E)-tetradec-7-ene. Heptanal (2.25 g) was converted to (E)-tetradec-7-ene in a manner similar to previous examples (see, e.g., Compound VI, step 4) to give 2.20 g of desired product.

Step 2: Ethyl 2-(2,3-dihexylcyclopropyl)-2-oxoacetate. (E)-tetradec-7-ene (1.1 g) was converted to ethyl 2-(2,3-dihexylcyclopropyl)-2-oxoacetate in a manner similar to previous examples (see, e.g., Compound IV, step 1) to give 0.44 g of desired product.

Step 3: 2-(2,3-dihexylcyclopropyl)-2-oxoacetic acid. Ethyl 2-(2,3-dihexylcyclopropyl)-2-oxoacetate (50 mg) was converted to 2-(2,3-dihexylcyclopropyl)-2-oxoacetic acid in a manner similar to previous examples (see, e.g., Compound IV, step 2) to give 40 mg of desired product.

Step 4: Sodium 2-(2,3-dihexylcyclopropyl)-2-oxoacetate. 2-(2,3-dihexylcyclopropyl)-2-oxoacetic acid (40 mg) was converted to sodium 2-(2,3-dihexylcyclopropyl)-2-oxoacetate in a manner similar to previous examples (see, e.g., Compound I, step 7) to give 35 mg of the final product. ¹H NMR (400 MHz, Methanol-d4) δ 2.08 (t, J=4.1 Hz, 1H), 1.70-1.21 (m, 23H), 0.89 (dt, J=6.9, 3.9 Hz, 6H). ¹³C NMR (101 MHz, Methanol-d4) δ 204.56, 169.33, 32.81, 31.57, 29.28, 28.83, 27.32, 22.28, 21.71, 13.04. Appearance: white solid. Melting point 241-243° C.

### Compound XXV: Synthesis of sodium 2-(2,3-dihexylcyclopropyl)acetate

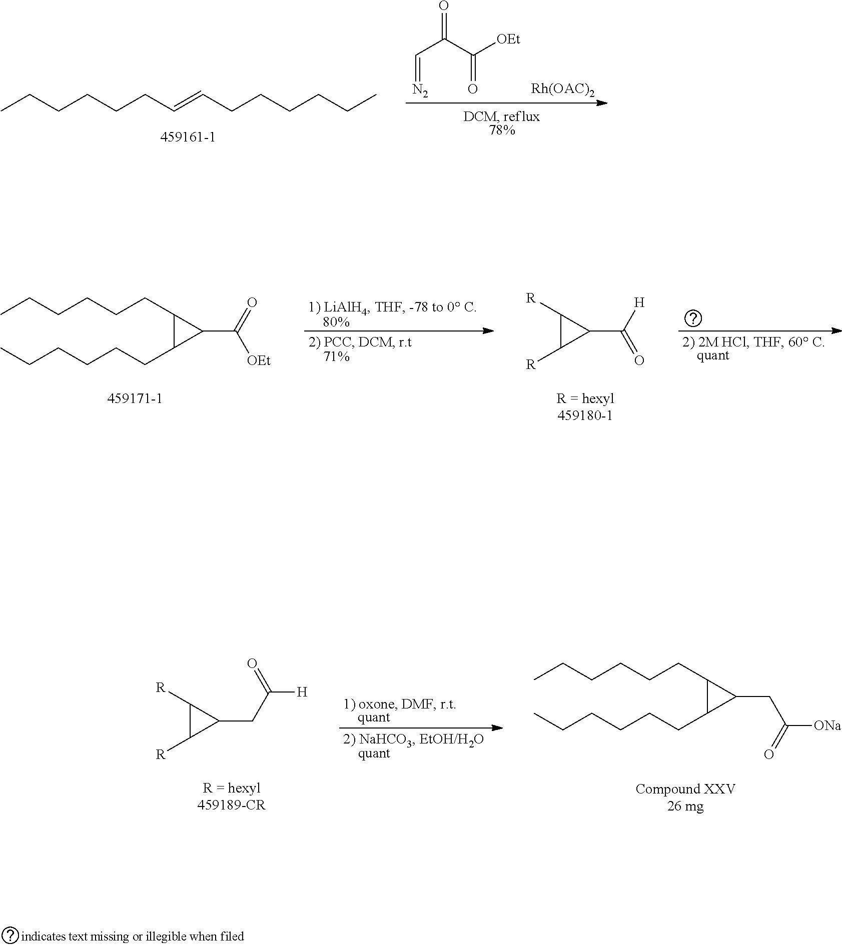

Step 1: Ethyl 2,3-dihexylcyclopropanecarboxylate. (E)-tetradec-7-ene (0.86 g) was converted to ethyl 2,3-dihexylcyclopropanecarboxylate in a manner similar to previous examples (see, e.g., Compound IV, step 1) to give 0.51 g of desired product.

Step 2: (2,3-dihexylcyclopropyl)methanol. Ethyl 2,3-dihexylcyclopropanecarboxylate (0.51 g) was converted to (2,3-dihexylcyclopropyl)methanol in a manner similar to previous examples (see, e.g., Compound I, step 2) to give 0.42 g of desired product.

Step 3: 2,3-dihexylcyclopropanecarbaldehyde. (2,3-dihexylcyclopropyl)methanol (0.42 g) was converted to 2,3-dihexylcyclopropanecarbaldehyde in a manner similar to previous examples (see, e.g., Compound IX, step 2) to give 0.33 g of desired product.

Step 4: (E)-1,2-dihexyl-3-(2-methoxyvinyl)cyclopropane. 2,3-dihexylcyclopropanecarbaldehyde (0.1 g) was converted to (E)-1,2-dihexyl-3-(2-methoxyvinyl)cyclopropane in a manner similar to previous examples (see, e.g., Compound IX, step 3) to give 33 mg of desired product.

Step 5: 2-(2,3-dihexylcyclopropyl)acetaldehyde. (E)-1,2-dihexyl-3-(2-methoxyvinyl) cyclopropane (33 mg) was converted to 2-(2,3-dihexylcyclopropyl)acetaldehyde in manner similar to previous examples to give 30 mg of desired product.

Step 6: 2-(2,3-dihexylcyclopropyl)acetic acid. 2-(2,3-dihexylcyclopropyl)acetaldehyde (30 mg) was converted to 2-(2,3-dihexylcyclopropyl)acetic acid in a manner similar to previous examples to give 30 mg of desired product.

Step 7: Sodium 2-(2,3-dihexylcyclopropyl)acetate. 2-(2,3-dihexylcyclopropyl)acetic acid (30 mg) was converted to sodium 2-(2,3-dihexylcyclopropyl)acetate in a manner similar to previous examples (see, e.g., Compound I, step 7) to give 26 mg of the final product. ¹H NMR (400 MHz, Methanol-d4) δ 2.05 (d, J=6.6 Hz, 2H), 1.48-1.20 (m, 21H), 0.89 (t, J=6.8 Hz, 6H), 0.57-0.43 (m, 2H). ¹³C NMR (101 MHz, Methanol-d4) δ 181.19, 42.84, 31.70, 29.87, 29.14, 28.15, 22.99, 22.35, 22.30, 13.08. Appearance: beige film.

### Compound XV: Synthesis of sodium 2,3-dihexylcyclopropanecarboxylate

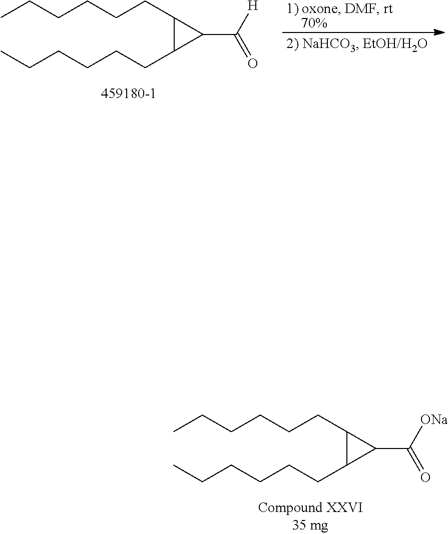

Step 1: 2,3-dihexylcyclopropanecarboxylic acid. 2,3-dihexylcyclopropanecarbaldehyde (66 mg) was converted to 2,3-dihexylcyclopropanecarboxylic acid in a manner similar to previous examples (see, e.g., Compound XXV, step 6) to give 47 mg of desired product.

Step 2: Sodium 2,3-dihexylcyclopropanecarboxylate. 2,3-dihexylcyclopropanecarboxylic acid (47 mg) was converted to sodium 2,3-dihexylcyclopropanecarboxylate in a manner similar to previous examples (see, e.g., Compound I, step 7) to give 45 mg of final product. ¹H NMR (400 MHz, Methanol-d4) δ 1.88-1.54 (m, 1H), 1.46-1.17 (m, 20H), 0.97-0.84 (n, 6H). ¹³C NMR (101 MHz, Methanol-d4) δ 182.52, 31.68, 30.20, 29.63, 28.96, 27.53, 25.88, 22.31, 13.06. Appearance: beige gum.

### Compounds XXVII-XXX: Synthesis of sodium 3-(2,2-dibromo-3-pentylcyclopropyl)propanoate, 3-(2,2-dimethyl-3-pentylcyclopropyl)propanoate, 2-(3-hexyl-2,2-dimethylcyclopropyl)acetate and 2-(3,3-[²H]₂-2-hexylcyclopropyl)acetate

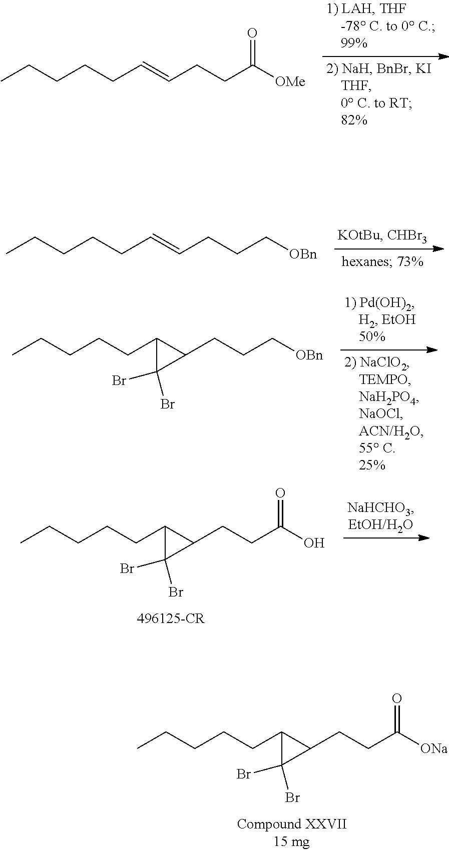

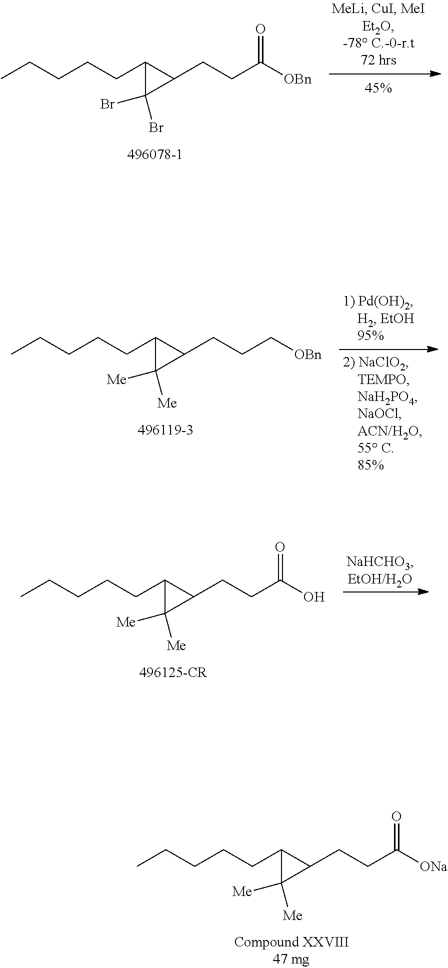

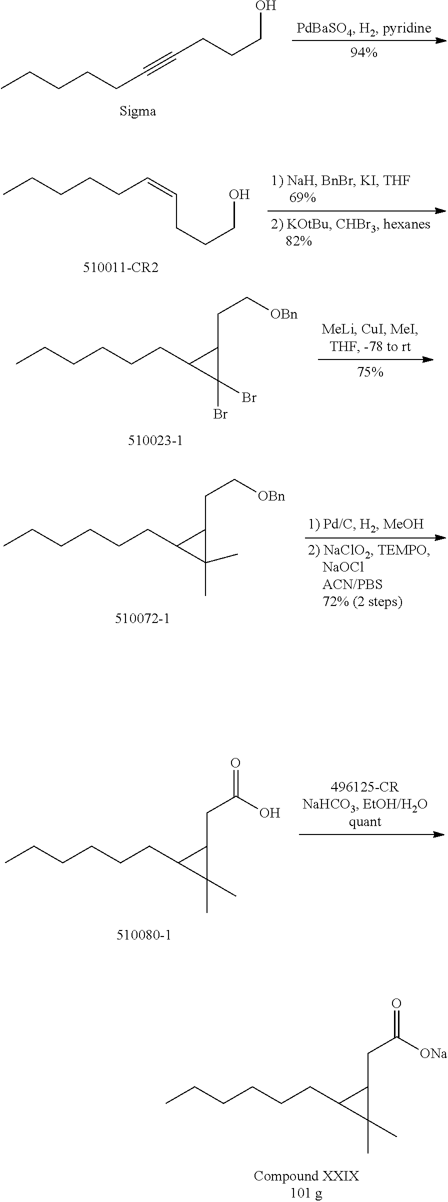

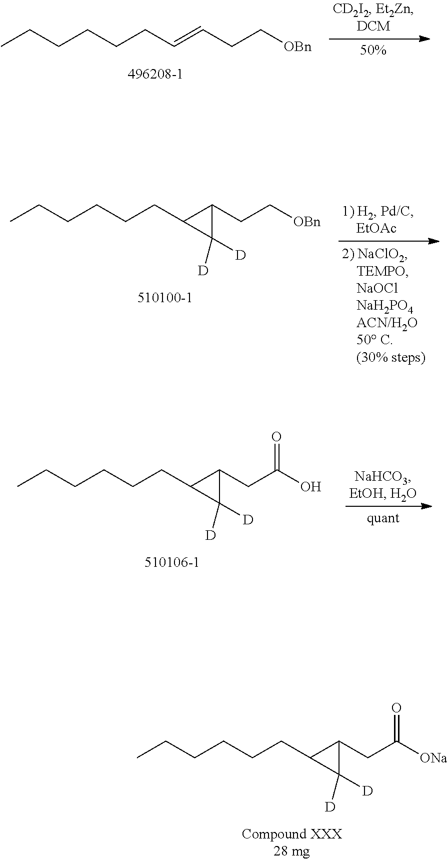

Step 1: (E)-dec-4-en-1-od. Methyl (E)-dec-4-enoate (5.0 g, 1 eq) was dissolved in dry THF (100 mL) and cooled to −78° C. LiAlH₄ (1.34 g, 1.3 eq) was then added in three portions over 5 minutes. Once the addition was complete the reaction was stirred at −78° C. for 30 minutes. At this point the reaction was warmed to 0° C. and stirred for an additional 30 minutes. EtOAc (10 mL) was then added to quench the reaction followed by a half-saturated solution of Rochelle's salt (150 mL). More EtOAc was then added and the mixture was warmed to room temperature and stirred vigorously for several hours. The layers were separated and the aqueous layer was extracted thrice more with EtOAc. Organic layers were combined, washed with brine and dried over Na₂SO₄. Concentration in vacuo gave 4.16 g of a colorless oil in (99% yield). ¹H NMR (400 MHz, Chloroform-d) δ 5.63-5.25 (m, 2H), 3.65 (t, J=6.5 Hz, 2H), 2.17-2.02 (m, 2H), 2.02-1.91 (m, 2H), 1.70-1.53 (m, 3H), 1.37-1.22 (m, 6H), 0.97-0.84 (m, 3H).

Step 2: (E)-((dec-4-enyloxy)methyl)benzene. This compound was prepared as for Compound I, step 3 to give 5.4 g (82% yield) of clean product. ¹H NMR (400 MHz, Chloroform-d) δ 7.50-7.17 (m, 5H), 5.57-5.25 (m, 2H), 4.50 (s, 2H), 3.47 (t, J=6.6 Hz, 2H), 2.21-2.02 (m, 2H), 2.01-1.90 (m, 2H), 1.68 (p, J=6.7 Hz, 2H), 1.28 (m, 6H), 0.94-0.83 (m, 3H).

Step 3: ((3-(2,2-dibromo-3-pentylcyclopropyl)propoxy)methyl)benzene. This compound was prepared as for Compound II, step 1 to give to give 2.5 g (73%) of the desired product. ¹H NMR (400 MHz, Chloroform-d) δ 7.63-7.19 (m, 5H), 4.52 (d, J=0.9 Hz, 2H), 3.53 (td, J=6.3, 4.3 Hz, 2H), 1.97-1.63 (m, 3H), 1.51-1.36 (m, 6H), 1.16-1.03 (m, 3H), 0.90 (t, J=6.6 Hz, 3H).

Step 4: 3-(2,2-dibromo-3-pentylcyclopropyl)propan-1-ol. This compound was prepared as for Compound I, step 5 to give to give 0.1 g (50%) of the desired product. ¹H NMR (400 MHz, Chloroform-d) δ 3.70 (t, J=6.3 Hz, 2H), 1.89-1.67 (m, 2H), 1.67-1.52 (m, 4H), 1.52-1.37 (m, 2H), 1.37-1.26 (m, 4H), 1.09 (ddd, J=6.2, 4.7, 1.7 Hz, 2H), 0.96-0.83 (m, 3H).

Step 5: 3-(2,2-dibromo-3-pentylcyclopropyl)propanoic acid. This compound was prepared as for Compound I, step 7 to give 24 mg (25% yield) of a colorless oil after purification.

¹H NMR (400 MHz, Chloroform-d) δ 2.70-2.47 (m, 2H), 1.87 (tq, J=14.4, 7.1 Hz, 2H), 1.72-1.55 (m, 1H), 1.55-1.37 (m, 3H), 1.37-1.26 (m, 4H), 1.22-1.08 (m, 2H), 1.02-0.80 (m, 3H).

Step 6: Sodium 3-(2,2-dibromo-3-pentylcyclopropyl)propanoate. This compound was prepared as for Compound I, step 5 to give a quantitative yield of clean product as a flaky white solid. ¹H NMR (400 MHz, Methanol-d₄) δ 2.56-2.19 (m, 2H), 2.02-1.81 (m, 1H), 1.72 (m, 1H), 1.62-1.43 (m, 4H), 1.43-1.28 (m, 4H), 1.26-1.10 (m, 2H), 1.01-0.82 (m, 3H); ¹³C NMR (101 MHz, Methanol-d₄) δ 179.99, 38.39, 36.88, 36.58, 35.84, 32.30, 31.27, 29.42, 27.65, 22.21, 12.93; MP: 185-190° C.

Step 1B: ((3-(2,2-dimethyl-3-pentylcyclopropyl)propoxy)methyl)benzene. A solution of MeLi (12.3 mL, 3.1 M in DME, 16 eq)) was added to a suspension of flame-dried CuI (3.6 g, 8 eq) in Et₂O (25 mL) at −78° C. This stirred mixture was allowed to briefly warm to 0° C. until the solution became homogeneous (approx. 5 minutes) then re-cooled to −78° C. A solution of ((3-(2,2-dibromo-3-pentylcyclopropyl)propoxy)methyl)benzene (in 5 mL Et₂O) was then added dropwise and the resultant solution was stirred at 0° C. for 72 hours. Mel (1.2 mL, 8 eq) was then added and the mixture was stirred at room temperature for an additional 24 hours. The reaction was then quenched with a saturated solution of NH₄C and extracted 3× with Et₂O. Organic layers were combined, washed with brine and dried over Na₂SO₄. Concentration in vacuo gave a brown oil that was purified on silica gel using Et₂O/hexanes to give 0.31 g (45%) of the desired product as a colorless oil. ¹H NMR (400 MHz, Chloroform-d) δ 7.56-7.15 (m, 5H), 4.50 (s, 2H), 3.48 (t, J=6.7 Hz, 2H), 1.72-1.63 (m, 2H), 1.50-1.36 (m, 1H), 1.37-1.09 (m, 9H), 0.99 (d, J=5.2 Hz, 6H), 0.93-0.77 (m, 3H), 0.15-−0.01 (m, 2H).

Step 2B: 3-(2,2-dimethyl-3-pentylcyclopropyl)propan-1-ol was prepared from ((3-(2,2-dimethyl-3-pentylcyclopropyl)propoxy)methyl)benzene in a manner similar to that described above (see, e.g., Compound I, step 5) to give 0.20 g (94%) of the desired product as a colorless oil. ¹H NMR (400 MHz, Chloroform-d) δ 3.66 (t, J=6.7 Hz, 2H), 1.70-1.53 (m, 3H), 1.47-1.11 (m, 9H), 1.00 (d, J=2.9 Hz, 6H), 0.94-0.80 (m, 3H), 0.18-−0.01 (m, 2H).

Step 3B: 3-(2,2-dimethyl-3-pentylcyclopropyl)propanoic acid was prepared from 3-(2,2-dimethyl-3-pentylcyclopropyl)propan-1-ol in a manner similar to that described above (see, e.g., Compound I, step 6) and was purified using HPLC (ACN/H₂O) to give 50 mg (25%) of the desired product as a colorless oil. ¹H NMR (400 MHz, Chloroform-d) δ 2.40 (t, J=7.6 Hz, 2H), 1.86-1.65 (m, 1H), 1.64-1.46 (m, 1H), 1.48-1.12 (m, 10H), 1.00 (d, J=8.6 Hz, 6H), 0.93-0.82 (m, 3H), 0.23-0.04 (m, 2H).

Step 4B: Sodium 3-(2,2-dimethyl-3-pentylcyclopropyl)propanoate was prepared from 3-(2,2-dimethyl-3-pentylcyclopropyl)propanoic acid in a manner similar to that described above (see, e.g., Compound I, step 7) to give the desired product as a sticky white solid in quantitative yield. ¹H NMR (400 MHz, Methanol-d₄) δ2.21 (t, J=7.9 Hz, 2H), 1.73-1.47 (m, 2H), 1.47-1.16 (m, 9H), 1.02 (d, J=13.4 Hz, 6H), 0.97-0.86 (m, 3H), 0.23-0.05 (m, 2H); ¹³C NMR (101 MHz, Methanol-d₄) δ 181.31, 38.21, 31.66, 31.64, 30.80, 29.74, 29.16, 26.44, 26.33, 22.37, 20.92, 18.84, 13.06; MP: 175-178° C.

Step 1C: (Z)-dec-3-en-1-ol. Dec-3-yn-1-ol (5.0 g, 1 eq) was dissolved in pyridine (20 mL) at room temperature and the solution was degassed via nitrogen balloon. PdBaSO₄ (5 wt %) was added and degassing is continued for several minutes. The reaction vessel was then sealed and hydrogen was introduced into the mixture via balloon. The reaction was then left to stir under hydrogen atmosphere for 12 hours. The reaction mixture was then filtered through Celite™ and concentrated in vacuo to give 4.69 g (94%) of the desired product as a yellow oil that was used without further purification. ¹H NMR (400 MHz, Chloroform-d) δ 5.69-5.43 (m, 1H), 5.43-5.11 (m, 1H), 3.63 (t, J=6.5 Hz, 2H), 2.44-2.27 (m, 2H), 2.05 (q, J=6.7 Hz, 2H), 1.56 (s, 1H), 1.44-1.18 (m, 8H), 0.96-0.78 (m, 3H).

Step 2C: (Z)-((dec-3-enyloxy)methyl)benzene was prepared from (Z)-dec-3-en-1-ol in a manner similar to that described above (see, e.g., Compound I, step 3). 4.8 g (68%) of desired product obtained as a yellow oil. ¹H NMR (400 MHz, Chloroform-d) δ 7.49-6.86 (m, 5H), 5.54-5.43 (m, 1H), 5.43-5.34 (m, 1H), 4.52 (s, 2H), 3.48 (t, J=7.1 Hz, 2H), 2.43-2.34 (m, 2H), 2.09-1.98 (m, 2H), 1.40-1.21 (m, 8H), 0.95-0.82 (m, 3H).

Step 3C: ((2-(2,2-dibromo-3-hexylcyclopropyl)ethoxy)methyl)benzene was prepared from (Z)-((dec-3-enyloxy)methyl)benzene in a manner similar to that described above (see, e.g., Compound II, step 1). 6.75 g (82%) of desired product were obtained as a faintly brown oil. ¹H NMR (400 MHz, Chloroform-d) δ 7.61-7.23 (m, 5H), 4.56 (s, 2H), 3.77-3.34 (m, 2H), 1.82-1.65 (m, 2H), 1.60 (dt, J=10.6, 6.7 Hz, 1H), 1.55-1.19 (m, 11H), 0.94-0.82 (m, 3H).

Step 4C: ((2-(3-hexyl-2,2-dimethylcyclopropyl)ethoxy)methyl)benzene was prepared from (2-(2,2-dibromo-3-hexylcyclopropyl)ethoxy)methyl)benzene in a manner similar to that described above (see, e.g., Compound III, step 1). 2.4 g (75%) of desired product were obtained as a colorless oil. ¹H NMR (400 MHz, Chloroform-d) δ 7.45-7.12 (m, 5H), 4.52 (d, J=1.3 Hz, 2H), 3.71-3.21 (m, 2H), 1.72-1.47 (m, 2H), 1.38-1.08 (m, 10H), 1.01 (s, 3H), 0.95-0.79 (m, 6H), 0.43 (qd, J=9.0, 4.7 Hz, 2H).

Step 5C: 2-(3-hexyl-2,2-dimethylcyclopropyl)ethanol was prepared from ((2-(3-hexyl-2,2-dimethylcyclopropyl)ethoxy)methyl)benzene in a manner similar to that described above (see, e.g., Compound I, step 5). 1.2 g (72%) of desired product was obtained as a colorless oil. ¹H NMR (400 MHz, Chloroform-d) δ 3.65 (t, J=6.9 Hz, 2H), 1.51 (qd, J=6.9, 3.7 Hz, 2H), 1.37-1.10 (m, 10H), 1.02 (s, 3H), 0.90 (d, J=14.4 Hz, 6H), 0.57-0.30 (m, 2H).

Step 6C: 2-(3-hexyl-2,2-dimethylcyclopropyoacetic acid was prepared from 2-(3-hexyl-2,2-dimethylcyclopropyl)ethanol in a manner similar to that described above (see, e.g., Compound I, step 6). 1.12 g (87%) of the desired product was obtained as a colorless oil. ¹H NMR (400 MHz, Chloroform-d) δ 2.45-2.23 (m, 2H), 1.39-1.12 (m, 10H), 1.06 (s, 3H), 0.92 (s, 3H), 0.91-0.85 (m, 3H), 0.82 (dt, J=8.9, 7.4 Hz, 1H), 0.60-0.48 (m, 1H).

Step 7C: Sodium 2-(3-hexyl-2,2-dimethylcyclopropyl)acetate was prepared from 2-(3-hexyl-2,2-dimethylcyclopropyl)acetic acid in a manner similar to that described above (see, e.g., Compound I, step 7). The desired product was obtained as a beige solid in quantitative yield. ¹H NMR (400 MHz, Methanol-d₄) δ 2.10 (d, J=7.4 Hz, 2H), 1.43-1.14 (m, 10H), 1.04 (s, 3H), 0.93 (s, 3H), 0.92-0.81 (m, 4H), 0.50-0.38 (m, 1H). ¹³C NMR (101 MHz, Methanol-d₄) δ 181.52, 32.60, 31.70, 29.96, 29.11, 28.29, 26.34, 24.18, 23.10, 22.34, 16.29, 13.93, 13.04. MP: 152-155° C.

Step 1 D: ((2-(3,3-[²H]₂-2-hexylcyclopropyl)ethoxy)methyl)benzene. (E)-((dec-3-enyloxy)methyl) benzene was dissolved in toluene and cooled to 0° C. under N₂ atmosphere. CD₂I₂ was added and then Et₂Zn (1.0 M in THF) was added dropwise over 30 minutes. Once the addition was complete the reaction is allowed to stir at room temperature for 2 hours. At this time the reaction is quenched by the addition of a saturated solution of NH₄Cl and extracted 3 times with Et₂O. The organic layers were combined, washed with brine and dried over Na₂SO₄. Concentration and purification on silica gel with Et₂O in hexanes gave the desired product as a colorless oil.

Step 2D: 2-(3,3-[²H]₂-2-hexylcyclopropyl)ethanol was prepared ((2-(3,3-[²H]₂-hexylcyclopropyl)ethoxy)methyl)benzene in the same manner as above (see, e.g., Compound I, step 5) to give the desired product as colorless oil.

Step 3D: 2-(3,3-[²H]₂-2-hexylcyclopropyl)acetic acid was prepared 2-(3,3-[²H]₂-2-hexylcyclopropyl)ethanol in a manner similar to that described above (see, e.g., Compound I, step 6) to give the desired product as a colorless oil.

Step 4D: Sodium 2-(3,3-[²H]₂-2-hexylcyclopropyl)acetate was prepared from 2-(3,3-[H]₂-2-hexylcyclopropyl)acetic acid in a similar manner to that described above (see, e.g., Compound I, step 7) to give the desired product. ¹H NMR (400 MHz, CD₃OD): δ 2.12 (dd, J=6.7, 14.1 Hz, 1H), 1.97 (dd, J=7.4, 14.1 Hz, 1H), 1.24-1.41 (m, 10H), 0.89 (t, J=6.8 Hz, 3H), 0.74-0.79 (m, 1H), 0.46-0.50 (m, 1H); ¹³C NMR (101 MHz, CD₃OD): δ 181.12, 42.48, 33.90, 31.70, 29.25, 28.96, 22.33, 18.02, 15.42, 13.05, 10.37 (pentet, J_{CD}=24.6 Hz); mp 227-230° C.

### Compound XXXI: Synthesis of sodium 3-(2,2-difluoro-3-pentylcyclopropyl)propanoate

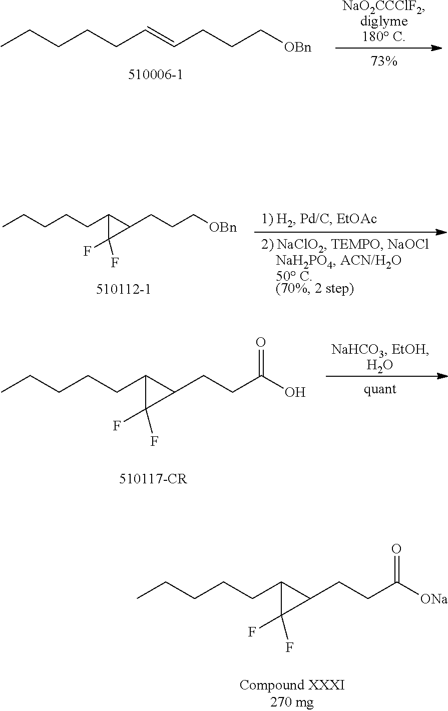

Sodium 3-(2,2-difluoro-3-pentylcyclopropyl)propanoate was prepared in the same manner as Compound I to give 0.28 g of final product. ¹H NMR (400 MHz, Methanol-d4) δ 2.37-2.12 (m, 2H), 1.77 (dq, J=13.8, 7.2 Hz, 1H), 1.70-1.54 (m, 1H), 1.47-1.24 (m, 9H), 1.24-1.05 (m, 2H), 0.99-0.86 (m, 3H). ¹³C NMR (101 MHz, Methanol-d4) δ 180.26, 119.57, 116.71, 113.85, 36.68, 31.14, 28.37, 28.23, 28.14, 28.04, 27.94, 26.33, 26.30, 23.43, 23.40, 22.17, 12.96. Appearance: white solid.

### Compound XXXII: Synthesis of sodium 2-(3-(5-hydroxyhexyl)-2,2-dimethylcyclopropyl)acetate

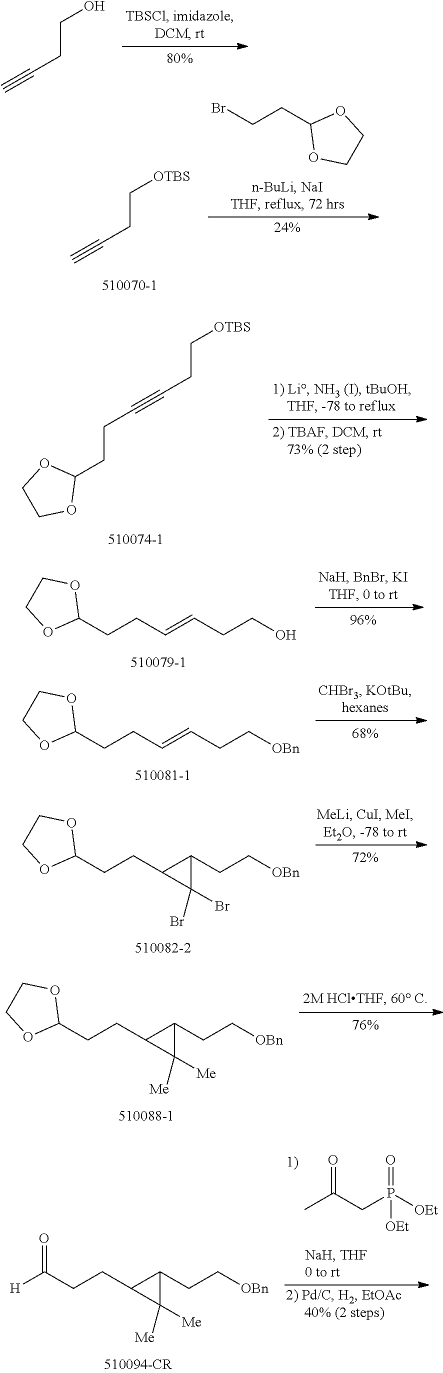

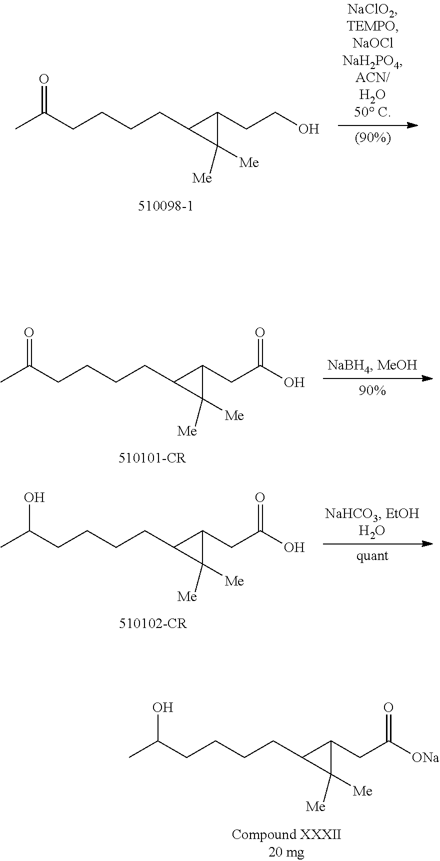

Step 1: (but-3-ynyloxy)(tert-butyl)dimethylsilane. A solution of but-3-yn-1-ol (4.1 g) and imidazole (8.3 g) in dichloromethane (200 ml), was treated with chloro(tert-butyl)dimethylsilane (11.5 g), and the reaction was stirred at ambient temperature for 5 hours. The reaction mixture was then diluted with water, and the layers were separated. The aqueous phase was further extracted with dichloromethane, and the combined organic extracts were washed with aqueous ammonium chloride solution (2 L), then dried over sodium sulfate and concentrated in vacuo to give the crude product. Purification by dry-flash chromatography on silica gel, eluting with 5% diethyl ether in hexanes, gave 8.0 g (75%) of desired product.

Step 2: (6-(1,3-dioxolan-2-yl)hex-3-ynyloxy)(tert-butyl)dimethylsilane. A solution of (but-3-ynyloxy)(tert-butyl)dimethylsilane (8.0 g) in tetrahydrofuran (100 ml) at −78° C., was treated dropwise with a commercial solution of n-butyllithium in hexanes (2.5 M, 20 ml), then allowed to warm to ambient temperature. The reaction mixture was cooled to 0° C., then treated with potassium iodide (1.6 g), and with a solution of 2-(2-bromoethyl)-1,3-dioxolane (7.5 g) in tetrahydrofuran (25 ml). The reaction was stirred at ambient temperature for 30 min, then refluxed at 50° C. for three days. The reaction was cooled to ambient temperature; quenched by gradual addition of water; then extracted with ethyl acetate. The organic extract was dried over sodium sulfate and concentrated in vacuo to give the crude product. Purification by silica gel chromatography, eluting with 0 to 10% ethyl acetate in hexanes, gave 2.30 g (24%) of desired product.

Step 3: (E)-(6-(1,3-dioxolan-2-yl)hex-3-enyloxy)(tert-butyl)dimethylsilane. Lithium wire (0.29 g) was added to liquid ammonia at −78° C., and the reaction was stirred at −78° C. for several minutes. A solution of (6-(1,3-dioxolan-2-yl)hex-3-ynyloxy)(tert-butyl)dimethylsilane (2.30 g) in tetrahydrofuran (4 ml) and tert-butanol (1.5 ml) was added dropwise; the cooling bath was then removed, and the reaction was allowed to warm to reflux. After 20 min the reaction was quenched by addition of a mixture of water, methanol and ethyl acetate, and ammonia was allowed to evaporate overnight. The layers were separated, and the aqueous phase was further extracted with ethyl acetate. Combined organic extracts were washed with saturated aqueous sodium chloride solution; then concentrated in vacuo to give the crude product (2.0 g) which was used in the next step without further purification.

Step 4: (E)-6-(1,3-dioxolan-2-yl)hex-3-en-1-ol. A solution of (E)-(6-(1,3-dioxolan-2-yl)hex-3-enyloxy)(tert-butyl)dimethylsilane (2.0 g) in tetrahydrofuran (15 ml) was treated slowly with a solution of tetrabutylammonium fluoride in tetrahydrofuran (1.0 M; 12 ml), and the reaction was stirred at ambient temperature for 2.5 hours. Water was then added, and the mixture was extracted with ethyl acetate. The organic extract was washed with saturated aqueous sodium chloride solution; dried over sodium sulfate; and concentrated in vacuo to give the crude product. Purification by silica gel chromatography, eluting with 0 to 50% ethyl acetate in hexanes, gave 1.00 g (73%) of desired product.

Step 5: (E)-2-(6-(benzyloxy)hex-3-enyl)-1,3-dioxolane. (E)-6-(1,3-dioxolan-2-yl)hex-3-en-1-ol (1.0 g) was converted to (E)-2-(6-(benzyloxy)hex-3-enyl)-1,3-dioxolane in a manner similar to previous examples (see, e.g., Compound I, step 3) to give 1.46 g of desired product.

Step 6: 2-(2-(3-(2-(benzyloxy)ethyl)-2,2-dibromocyclopropyl)ethyl)-1,3-dioxolane. (E)-2-(6-(benzyloxy)hex-3-enyl)-1,3-dioxolane (1.46 g) was converted to 2-(2-(3-(2-(benzyloxy)ethyl)-2,2-dibromocyclopropyl)ethyl)-1,3-dioxolane in a manner similar to previous examples (see, e.g., Compound II, step 1) to give 1.05 g of desired product.

Step 7: 2-(2-(3-(2-(benzyloxy)ethyl)-2,2-dimethylcyclopropyl)ethyl)-1,3-dioxolane. 2-(2-(3-(2-(benzyloxy)ethyl)-2,2-dibromocyclopropyl)ethyl)-1,3-dioxolane (1.05 g) was converted to 2-(2-(3-(2-(benzyloxy)ethyl)-2,2-dimethylcyclopropyl)ethyl)-1,3-dioxolane in a manner similar to previous examples (see, e.g., Compound III, step 1) to give 0.52 g of desired product.

Step 8: 3-(3-(2-(benzyloxy)ethyl)-2,2-dimethylcyclopropyl)propanal. 2-(2-(3-(2-(benzyloxy)ethyl)-2,2-dimethylcyclopropyl)ethyl)-1,3-dioxolane (0.52 g) was converted to 3-(3-(2-(benzyloxy)ethyl)-2,2-dimethylcyclopropyl)propanal in a manner similar to previous examples to give 0.34 g of desired product.

Step 9: (E)-6-(3-(2-hydroxyethyl)-2,2-dimethylcyclopropyl)hex-3-en-2-one. 3-(3-(2-(benzyloxy)ethyl)-2,2-dimethylcyclopropyl)propanal (0.10 g) was converted to (E)-6-(3-(2-hydroxyethyl)-2,2-dimethylcyclopropyl)hex-3-en-2-one in a manner similar to previous examples (see, e.g., Compound VI, step 4) to give 50 mg of desired product.

Step 10: 6-(3-(2-hydroxyethyl)-2,2-dimethylcyclopropyl)hexan-2-one. (E)-6-(3-(2-hydroxyethyl)-2,2-dimethylcyclopropyl)hex-3-en-2-one (50 mg) was converted to 6-(3-(2-hydroxyethyl)-2,2-dimethylcyclopropyl)hexan-2-one in a manner similar to previous examples (see, e.g., Compound I, step 5) to give 30 mg of desired product.

Step 11: 2-(2,2-dimethyl-3-(5-oxohexyl)cyclopropyl)acetic acid. 6-(3-(2-hydroxyethyl)-2,2-dimethylcyclopropyl)hexan-2-one (30 mg) was converted to 2-(2,2-dimethyl-3-(5-oxohexyl)cyclopropyl)acetic acid in a manner similar to previous examples (see, e.g., Compound I, step 6) to give 30 mg of desired product.

Step 12: 2-(3-(5-hydroxyhexyl)-2,2-dimethylcyclopropyl)acetic acid. A solution of 2-(2,2-dimethyl-3-(5-oxohexyl)cyclopropyl)acetic acid (30 mg) in methanol (10 ml) at 0° C. was treated portion-wise with sodium borohydride (0.01 g) over 5 minutes. The reaction was stirred at 0° C. for 90 min; then concentrated in vacuo; and the residue partitioned between ethyl acetate and water. The organic phase was washed with saturated aqueous sodium chloride solution; dried over sodium sulfate; and concentrated in vacuo to give the crude product. Purification by silica gel chromatography, eluting with 0 to 50% ethyl acetate in hexanes, gave 30 mg of desired product.

Step 13: Sodium 2-(3-(5-hydroxyhexyl)-2,2-dimethylcyclopropyl)acetate. 2-(3-(5-hydroxyhexyl)-2,2-dimethylcyclopropyl)acetic acid (30 mg) was converted to sodium 2-(3-(5-hydroxyhexyl)-2,2-dimethylcyclopropyl)acetate in a manner similar to previous examples (see, e.g., Compound I, step 7) to give 20 mg of final product. ¹H NMR (400 MHz, Methanol-d4) δ 3.69 (p, J=6.4, 5.7 Hz, 1H), 2.32-1.99 (m, 2H), 1.52-1.24 (m, 9H), 1.13 (d, J=6.2 Hz, 3H), 1.03 (d, J=3.1 Hz, 6H), 0.65-0.49 (m, 1H), 0.22 (tt, J=6.6, 3.6 Hz, 1H). ¹³C NMR (101 MHz, Methanol-d4) δ 181.57, 67.15, 67.11, 38.89, 38.88, 37.75, 30.48, 30.45, 29.95, 29.93, 29.09, 27.59, 25.45, 25.43, 22.06, 22.04, 21.10, 20.63, 18.45. Appearance: white film.

Compound XXXIII: Synthesis of sodium 8-(2,2-dimethylcyclopropyl)octanoate

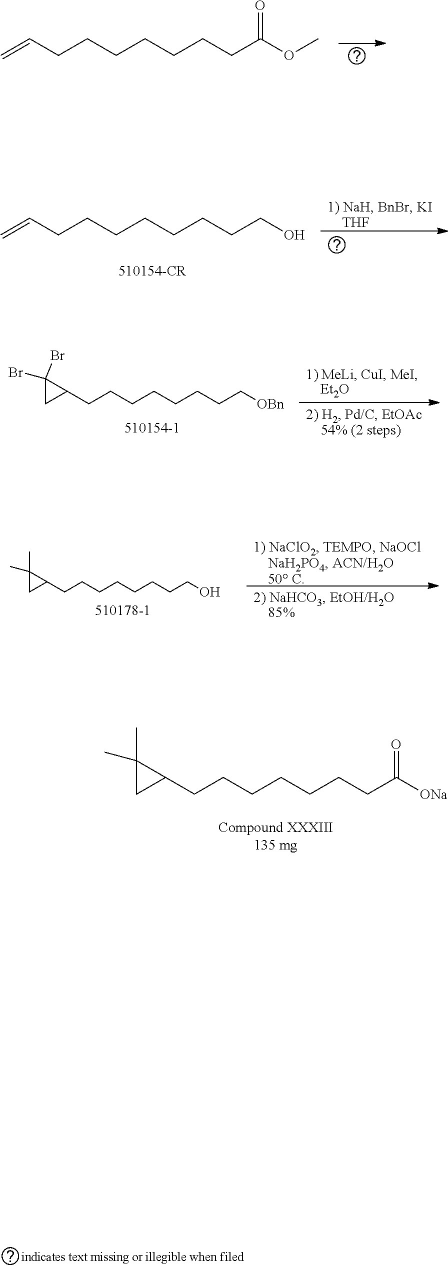

Step 1: dec-9-en-1-ol. Methyl dec-9-enoate (2.35 g) was converted to dec-9-en-1-ol in a manner similar to previous examples (see, e.g., Compound I, step 2) to give 1.96 g of desired product.

Step 2 ((dec-9-enyloxy)methyl)benzene. dec-9-en-1-ol (1.91 g) was converted to ((dec-9-enyloxy)methyl)benzene in a manner similar to previous examples (see, e.g., Compound I, step 3) to give 2.1 g of desired product.

Step 3: ((8-(2,2-dibromocyclopropyl)octyloxy)methyl)benzene. ((dec-9-enyloxy)methyl)benzene (1.0 g) was converted to ((8-(2,2-dibromocyclopropyl)-octyloxy)methyl)benzene in a manner similar to previous examples (see, e.g., Compound II, step 1) to give 1.34 g of desired product.

Step 4 ((8-(2,2-dimethylcyclopropyl)octyloxy)methyl)benzene. ((8-(2,2-dibromocyclopropyl)-octyloxy)methyl)benzene (1.34 g) was converted to ((8-(2,2-dimethylcyclopropyl)-octyloxy)methyl)benzene in a manner similar to previous examples (see, e.g., Compound III, step 1) to give 0.55 g of desired product.

Step 5: 8-(2,2-dimethylcyclopropyl)octan-1-ol. ((8-(2,2-dimethylcyclopropyl)-octyloxy)methyl)benzene (0.55 g) was converted to 8-(2,2-dimethyl-cyclopropyl)octan-1-ol in a manner similar to previous examples (see, e.g., Compound I, step 5) to give 0.31 g of desired product.

Step 6: 8-(2,2-dimethylcyclopropyl)octanoic acid. 8-(2,2-dimethyl-cyclopropyl)octan-1-ol was converted to 8-(2,2-dimethylcyclopropyl)octanoic acid in a manner similar to previous examples (see, e.g., Compound I, step 6) to give 0.25 g of desired product

Step 7: Sodium 8-(2,2-dimethylcyclopropyl)octanoate. 8-(2,2-dimethylcyclopropyl)octanoic acid (0.15 g) was converted to sodium 8-(2,2-dimethyl cyclopropyl)octanoate in a manner similar to previous examples (see, e.g., Compound I, step 7) to give 135 mg of final product. ¹H NMR (400 MHz, Methanol-d4) δ 2.21-2.10 (m, 2H), 1.67-1.50 (m, 2H), 1.44-1.20 (m, 10H), 1.02 (d, J=5.3 Hz, 6H), 0.54-0.41 (m, 1H), 0.34 (dd, J=8.5, 4.0 Hz, 1H), −0.07-−0.21 (m, 1H). Appearance: white solid. Melting point: 208-212° C.

### Compound XXXIV: Synthesis of sodium 2-(3-hexyloxiran-2-yl)acetate

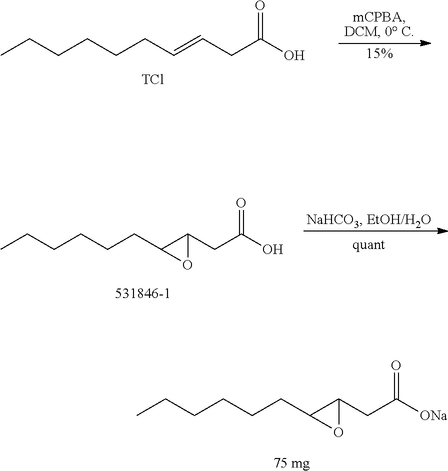

Step 1: 2-(3-hexyloxiran-2-yl)acetic acid. A solution of (E)-dec-3-enoic acid (0.5 g) in dichloromethane (25 ml) at 0° C., was treated with 4-chloroperbenzoic acid (77% w/w; 0.82 g), and the reaction was stirred at 0° C. for 1 hour. The reaction mixture was diluted in dichloromethane, then washed with aqueous sodium dihydrogenphosphate (pH 4.5), and with saturated aqueous sodium chloride; dried over sodium sulfate; and concentrated in vacuo to give the crude product. Purification by silica gel chromatography, eluting with 0 to 100% ethyl acetate in hexanes, gave 80 mg (15%) of desired product.

Step 2: Sodium 2-(3-hexyloxiran-2-yl)acetate. 2-(3-hexyloxiran-2-yl)acetic acid (80 mg) was converted to sodium 2-(3-hexyloxiran-2-yl)acetate in a manner similar to previous examples (see, e.g., Compound I, step 7) to give 75 mg of the final product. ¹H NMR (400 MHz, Methanol-d4) δ 3.04 (td, J=6.0, 2.3 Hz, 1H), 2.75 (td, J=6.1, 5.4, 2.2 Hz, 1H), 2.43-2.19 (m, 2H), 1.58 (dt, J=7.0, 5.7 Hz, 1H), 1.53-1.23 (m, 10H), 0.98-0.84 (m, 3H). ¹³C NMR (101 MHz, Methanol-d4) δ 177.27, 58.85, 56.23, 40.80, 31.68, 31.53, 28.84, 25.58, 22.21, 12.98. Appearance: white solid. Melting point: 134-136° C.

### Compound XXXV: Synthesis of trans-5-(3-(carboxymethyl)-2,2-dimethylcyclopropyl) pentanoic acid, disodium salt

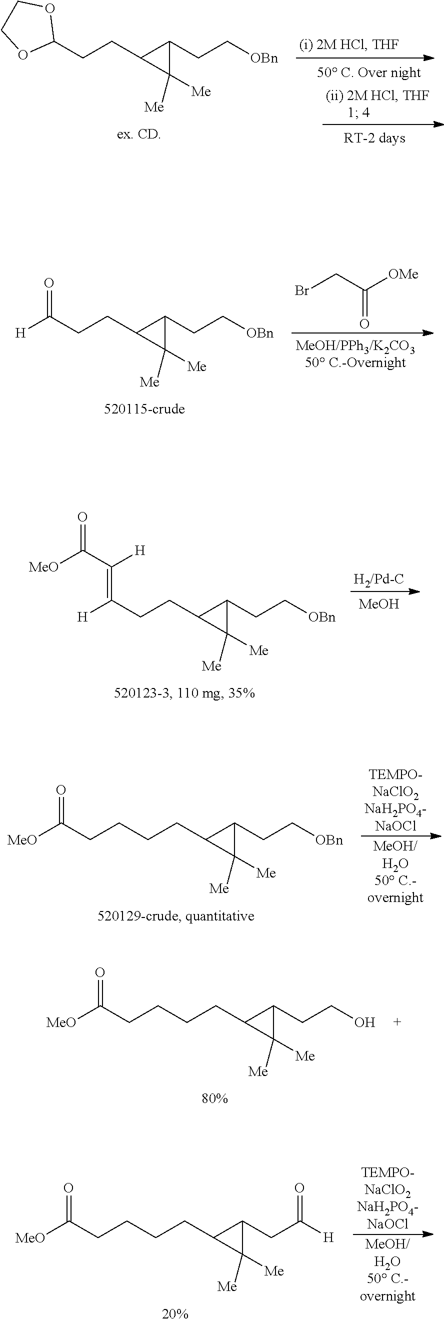

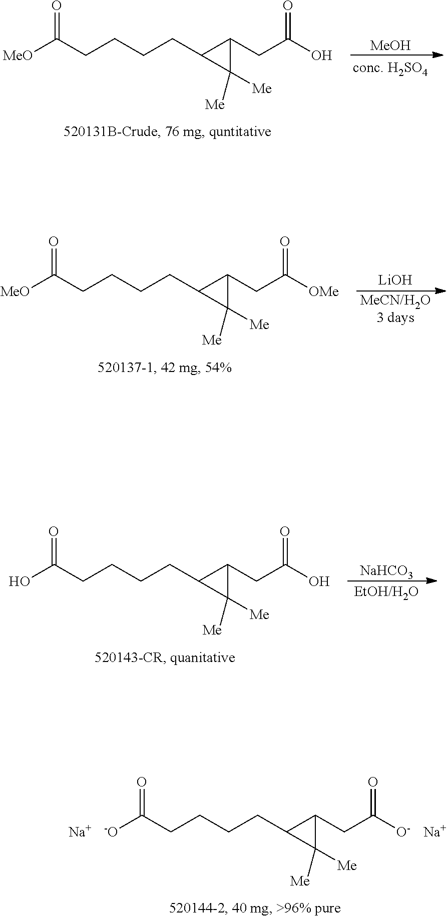

Step 1: 2-(2-(3-(2-(benzyloxy)ethyl)-2,2-dimethylcyclopropyl)ethyl)-1,3-dioxolane previously prepared (298 mg, 1 eq) was dissolved in THF (8.6 ml) and treated with 5 drops of 2M HCl. The reaction was stirred at 50° C. for 2.5 hours and then checked by NMR. The reaction was then left at 50° C. overnight. The reaction was diluted with 1M aq HCl and extracted 3 three times with EtOAC. The combined organic layers were washed with brine, dried over anhydrous Na₂SO₄ and concentrated in vacuum. ¹H-NMR showed a mixture of starting material and product. The reaction was repeated using (THF and 2M aq.HCl 4:1) for 2 days at RT giving 255 mg of crude product 3-(3-(2-(benzyloxy)ethyl)-2,2-dimethylcyclopropyl)propanal.

Step 2: 3-(3-(2-(benzyloxy)ethyl)-2,2-dimethylcyclopropyl)propanal (255 mg, 1 eq) was dissolved in 6 ml MeOH and treated with methyl bromoacetate (0.12 ml, 1.2 eq), Triphenylphosphine (313 mg, 1.2 eq) and Potassium carbonate (163 mg, 1.2 eq). The reaction mixture was stirred at 50° C. with reflux overnight for 24 hours. The reaction was cooled to RT, excess of methanol was evaporated, the reaction was diluted with H₂O and extracted twice with dichloromethane. The combined organic layers were washed with brine, dried over anhydrous Na₂SO₄ and concentrated in vacuum. Purification on column chromatography silica gel (0-4% EtOAc/Hexanes) gave 110 mg of product pure ((E)-methyl 5-(3-(2-(benzyloxy)ethyl)-2,2-dimethylcyclopropyl)pent-2-enoate (35% yield).

Step 3 ((E)-methyl 5-(3-(2-(benzyloxy)ethyl)-2,2-dimethylcyclopropyl) pent-2-enoate 102.5 mg was dissolved in methanol (0.64 ml) and degassed via N₂ balloon, Pd/C (10.25 mg) was then added and N₂ bubbling was continued for several minutes. Reaction was then sealed and H₂ was introduced via balloon. After bubbling H₂ through the reaction mixture for several minutes, the reaction was left to stir under H₂ atmosphere for 22 hours. At this point the reaction was opened to air and filtered through sand/Celite™. Concentration in vacuo gave a colorless oil 73 mg (100% yield) of the desired product (methyl 5-(3-(2-hydroxyethyl)-2,2-dimethylcyclopropyl)pentanoate).

Step 4: Methyl 5-(2-(3-(5-Methoxy-5-oxoethyl)-2,2-dimethylcyclopropyl)pentanoate was prepared as for Compound I, Step 6, to give the crude expected product: (2-(3-(5-methoxy-5-oxopentyl)-2,2-dimethylcyclopropyl)acetic acid.

Step 5: Methyl 5-(2-(3-(5-Methoxy-5-oxoethyl)-2,2-dimethylcyclopropyl)pentanoate was prepared as for Compound I, Step I, to give 42 mg of product pure (54% yield).

Step 6: 5-(3-(Carboxymethyl)-2,2-dimethylcyclopropyl)pentanoic acid was prepared as for Compound IV, Step 2.

Step 7: 5-(3-(Carboxymethyl)-2,2-dimethylcyclopropyl)pentanoic acid disodium salt was prepared as for Compound I, Step 7 (40 mg) (99% yield). ¹H NMR (400 MHz, Methanol-d₄) δ 2.23-2.09 (m, 4H), 1.60 (p, J=7.4 Hz, 2H), 1.39 (dt, J=7.0, 3.3 Hz, 3H), 1.34-1.24 (m, 1H), 1.03 (s, 3H), 1.02 (s, 3H), 0.56 (td, J=7.3, 5.5 Hz, 1H), 0.27-0.15 (m, 1H). ¹³C NMR (101 MHz, Methanol-d₄) δ 181.76, 181.65, 38.07, 37.86, 30.53, 30.20, 29.21, 27.52, 26.55, 21.12, 20.62, 18.52.

### Compound XXXVI: Synthesis of sodium (E)-8-(2,2-dimethylcyclopropyl)oct-2-enoate

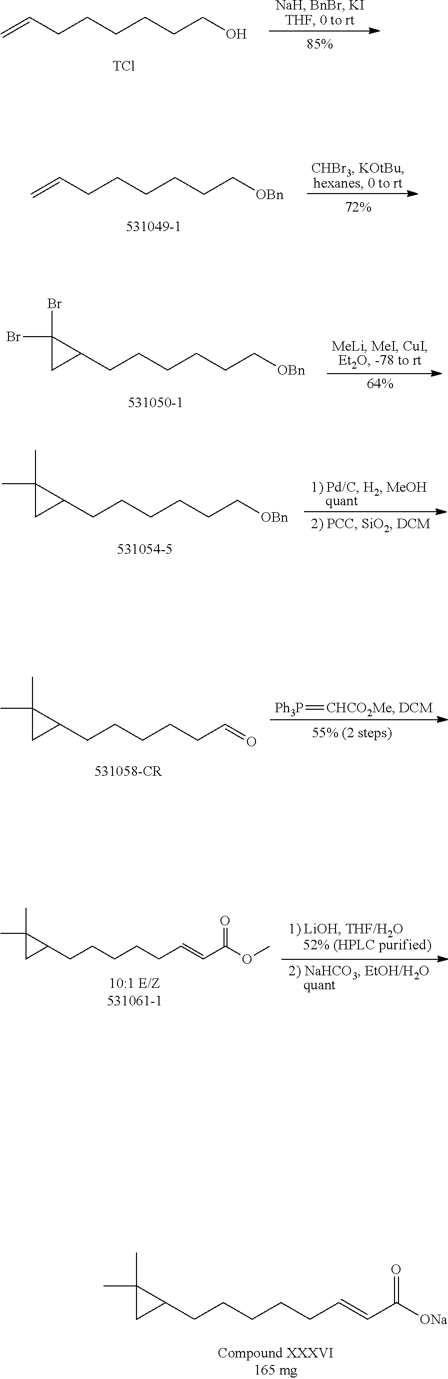

Step 1: ((oct-7-enyloxy)methyl)benzene. Oct-7-en-1-ol (4.65 g) is converted to ((oct-7-enyloxy)methyl)benzene in a manner similar to previous examples (see, e.g., Compound I, step 3) to give 6.62 g of desired product.

Step 2: ((6-(2,2-dibromocyclopropyl)hexyloxy)methyl)benzene. (oct-7-enyloxy)methyl)benzene (6.60 g) is converted to ((6-(2,2-dibromocyclopropyl)-hexyloxy)methyl)benzene in a manner similar to previous examples (see, e.g., Compound II, step 1) to give 8.5 g of desired product.

Step 3: ((6-(2,2-dimethylcyclopropyl)hexyloxy)methyl)benzene. ((6-(2,2-dibromocyclopropyl)-hexyloxy)methyl)benzene (8.5 g) is converted to ((6-(2,2-dimethylcyclopropyl)hexyloxy)methyl)benzene in a manner similar to previous examples (see, e.g., Compound III, step 1) to give 3.62 g of desired product.

Step 4: 6-(2,2-dimethylcyclopropyl)hexan-1-al. ((6-(2,2-dimethylcyclopropyl)hexyloxy)methyl)benzene (3.62 g) is converted to 6-(2,2-dimethylcyclopropyl)hexan-1-ol in a manner similar to previous examples (see, e.g., Compound I, step 5) to give 2.30 g of desired product.

Step 5: 6-(2,2-dimethylcyclopropyl)hexanal. 6-(2,2-dimethylcyclopropyl)hexan-1-ol (0.5 g) is converted to 6-(2,2-dimethylcyclopropyl)hexanal in a manner similar to previous examples (see, e.g., Compound IX, step 2) to give 0.5 g of desired product.

Step 6: (E)-methyl 8-(2,2-dimethylcyclopropyl)oct-2-enoate. 6-(2,2-dimethylcyclopropyl)hexanal (0.5 g) is converted to (E)-methyl 8-(2,2-dimethylcyclopropyl)oct-2-enoate in a manner similar to previous examples (see, e.g., Compound IX, step 3) to give 0.36 g of desired product.

Step 7: (E)-8-(2,2-dimethylcyclopropyl)oct-2-enoic acid. (E)-methyl 8-(2,2-dimethylcyclopropyl)oct-2-enoate (0.36 g) is converted to (E)-8-(2,2-dimethylcyclopropyl)oct-2-enoic acid in a manner similar to previous examples (see, e.g., Compound IV, step 2) to give 0.17 g of desired product.

Step 8: Sodium (E)-8-(2,2-dimethylcyclopropyl)oct-2-enoate. (E)-8-(2,2-dimethylcyclopropyl)oct-2-enoic acid (0.17 g) is converted to sodium (E)-8-(2,2-dimethylcyclopropyl)oct-2-enoate in a manner similar to previous examples (see, e.g., Compound I, step 7) to give 165 mg of final product. ¹H NMR (400 MHz, Methanol-d4) δ 6.60 (dt, J=15.5, 7.0 Hz, 1H), 5.81 (dt, J=15.5, 1.5 Hz, 1H), 2.13 (qd, J=7.2, 1.4 Hz, 2H), 1.36 (dddd, J=35.6, 17.3, 14.1, 7.3 Hz, 10H), 1.02 (d, J=5.3 Hz, 6H), 0.47 (ddd, J=8.5, 6.9, 5.4 Hz, 1H), 0.35 (dd, J=8.5, 4.0 Hz, 1H), −0.07-−0.23 (m, 1H). ¹³C NMR (101 MHz, Methanol-d4) δ 174.51, 142.68, 127.61, 31.55, 29.75, 29.39, 28.80, 28.41, 26.60, 24.54, 19.02, 18.87, 14.79. Appearance: white solid. Melting point: 220-221° C. UPLC/MS: 1-100% ACN(+0.01% FA) in 5 mins; r.t.=3.65 mins; ES(+): 211.2.

### Compound XXXVII: Synthesis of sodium 2-heptylcyclopropanecarboxylate

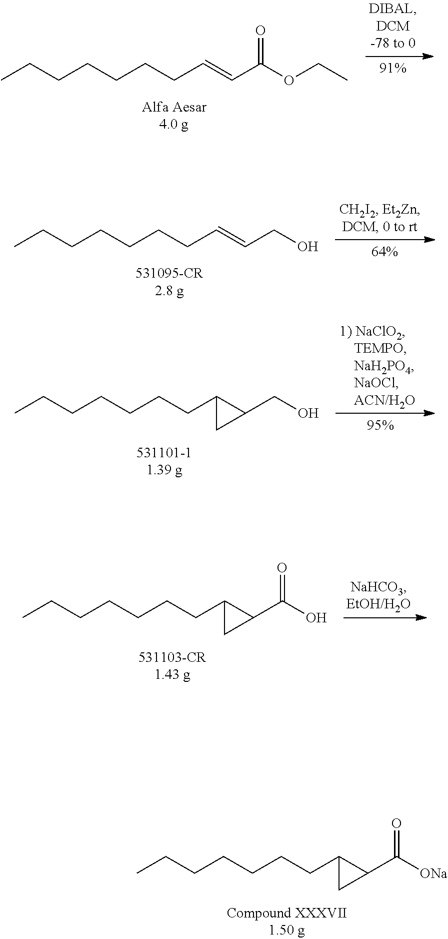

Step 1: (E)-dec-2-en-1-ol. (E)-ethyl dec-2-enoate (4.0 g) was converted to (E)-dec-2-en-1-ol in a manner similar to previous examples (see, e.g., Compound I, step 2) to give 2.80 g of desired product.

Step 2: (2-heptylcyclopropyl)methanol. (E)-dec-2-en-1-ol (2.0 g) was converted to (2-heptylcyclopropyl)methanol in a manner similar to previous examples (see, e.g., Compound VI, step 5) to give 1.39 g of desired product.

Step 3: 2-heptylcyclopropanecarboxylic acid. (2-heptylcyclopropyl)methanol (1.39 g) was converted to 2-heptylcyclopropanecarboxylic acid in a manner similar to previous examples (see, e.g., Compound I, step 6) to give 1.43 g of desired product.

Step 4: Sodium 2-heptylcyclopropanecarboxylate. 2-heptylcyclopropanecarboxylic acid (1.43 g) was converted to sodium 2-heptylcyclopropanecarboxylate in a manner similar to previous examples (see, e.g., Compound I, step 7) to give 1.5 g of the final product. ¹H NMR (400 MHz, Methanol-d4) δ 1.40 (q, J=7.6 Hz, 2H), 1.36-1.21 (m, 12H), 1.18 (ddd, J=9.6, 4.6, 3.0 Hz, 2H), 1.01-0.91 (m, 1H), 0.91-0.86 (m, 3H), 0.47-0.33 (m, 1H). ¹³C NMR (101 MHz, Methanol-d4) δ 182.17, 33.30, 31.61, 29.11, 29.10, 29.05, 22.99, 22.29, 20.63, 13.37, 13.00. Appearance: white film

### Compound XXXVIII: Synthesis of sodium 2-(3-cyclohexylpropyl)cyclopropanecarboxylate

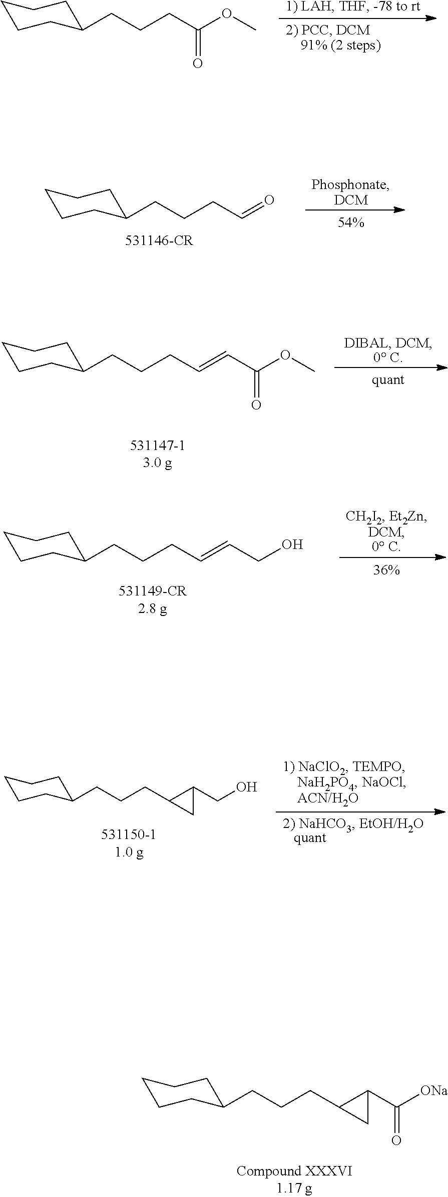

Step 1: 4-cyclohexylbutan-1-ol. Methyl 4-cyclohexylbutanoate (5.39 g) was converted to 4-cyclohexylbutan-1-ol in a manner similar to previous examples (see, e.g., Compound I, step 2) to give 4.25 g of desired product.

Step 2: 4-cyclohexylbutanal. 4-cyclohexylbutan-1-ol (4.40 g) was converted to 4-cyclohexylbutanal in a manner similar to previous examples (see, e.g., Compound IX, step 2) to give 4.25 g of desired product.

Step 3: (E)-methyl 6-cyclohexylhex-2-enoate. 4-cyclohexylbutanal (4.25 g) was converted to (E)-methyl 6-cyclohexylhex-2-enoate in a manner similar to previous examples (see, e.g., Compound IX, step 3) to give 3.33 g of desired product.

Step 4: (E)-6-cyclohexylhex-2-en-1-od. (E)-methyl 6-cyclohexylhex-2-enoate (3.03 g) was converted to (E)-6-cyclohexylhex-2-en-1-ol in a manner similar to previous examples (see, e.g., Compound I, step 2) to give 2.80 g of desired product.

Step 5: (2-(3-cyclohexylpropyl)cyclopropyl)methanol. (E)-6-cyclohexylhex-2-en-1-ol (2.80 g) was converted to (2-(3-cyclohexylpropyl)cyclopropyl)methanol in a manner similar to previous examples (see, e.g., Compound VI, step 5) to give 1.0 g of desired product.

Step 6: 2-(3-cyclohexylpropyl)cyclopropanecarboxylic acid. (2-(3-cyclohexylpropyl)cyclopropyl)methanol (1.0 g) was converted to 2-(3-cyclohexylpropyl)-cyclopropanecarboxylic acid in a manner similar to previous examples (see, e.g., Compound I, step 6) to give 1.12 g of desired product.

Step 7: Sodium 2-(3-cyclohexylpropyl)cyclopropanecarboxylate. 2-(3-cyclohexylpropyl)-cyclopropanecarboxylic acid (1.12 g) was converted to sodium 2-(3-cyclohexylpropyl)cyclopropanecarboxylate in a manner similar to previous examples (see, e.g., Compound I, step 7) to give 1.18 g of the final product. ¹H NMR (400 MHz, Methanol-d4) δ 1.75-1.60 (m, 6H), 1.41 (q, J=7.8, 6.7 Hz, 2H), 1.31-1.07 (m, 13H), 1.00-0.80 (m, 4H), 0.48-0.36 (m, 1H). Appearance: white solid. Melting point: 175-178° C. (decomp). UPLC/MS: 1-100% ACN(+0.01% FA) in 5 mins; r.t.=3.53 mins; ES(+): 193.0.

### Compound XXXIX: Synthesis of sodium 2-(3-m-tolylpropyl)-cyclopropanecarboxylate

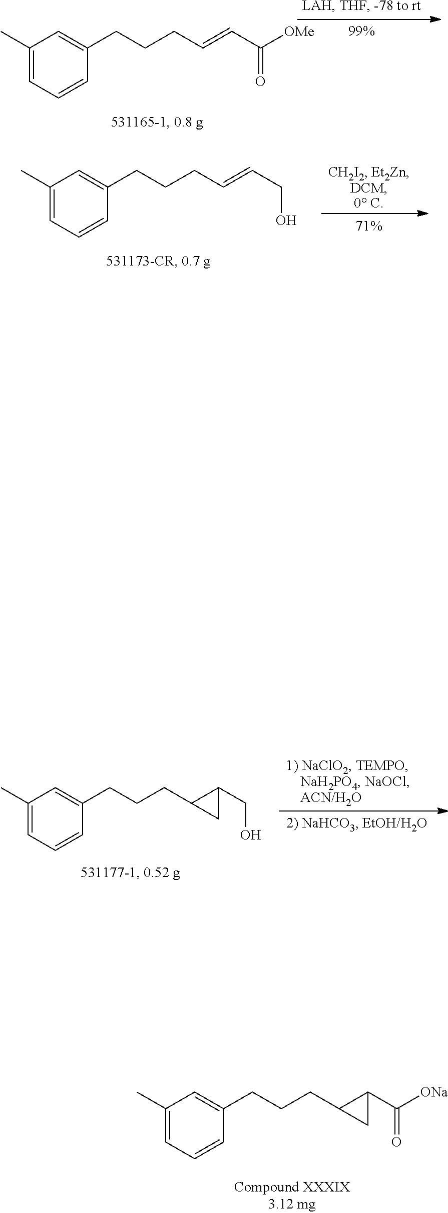

Step 1: (E)-6-m-tolylhex-2-en-1-o. (E)-methyl 6-m-tolylhex-2-enoate (0.8 g) was converted to (E)-6-m-tolylhex-2-en-1-ol in a manner similar to previous examples (see, e.g., Compound I, step 2) to give 0.7 g of the desired product.

Step 2: (2-(3-m-tolylpropyl)cyclopropyl)methanol. (E)-6-m-tolylhex-2-en-1-ol (0.70 g) was converted to (2-(3-m-tolylpropyl)cyclopropyl)methanol in a manner similar to previous examples (see, e.g., Compound VI, step 5) to give 0.52 g of desired product.

Step 3: 2-(3-m-tolylpropyl)cyclopropanecarboxylic acid. (2-(3-m-tolylpropyl)cyclopropyl)methanol (0.52 g) was converted to 2-(3-m-tolylpropyl)-cyclopropanecarboxylic acid in a manner similar to previous examples (see, e.g., Compound I, step 6) to give 0.33 g of desired product.

Step 4: Sodium 2-(3-m-tolylpropyl)cyclopropanecarboxylate. 2-(3-m-tolylpropyl)cyclopropanecarboxylic acid (0.32 g) was converted to sodium 2-(3-m-tolylpropyl)-cyclopropanecarboxylate in a manner similar to previous examples (see, e.g., Compound I, step 7) to give 0.32 g of the final product. ¹H NMR (400 MHz, Methanol-d4) δ 7.10 (t, J=7.5 Hz, 1H), 7.01-6.86 (m, 3H), 2.58 (t, J=7.7 Hz, 2H), 2.28 (s, 3H), 1.77-1.61 (m, 2H), 1.38-1.10 (m, 4H), 1.02-0.92 (m, 1H), 0.43 (ddd, J=8.8, 5.6, 3.6 Hz, 1H). ¹³C NMR (101 MHz, Methanol-d4) δ 182.04, 142.36, 137.32, 128.67, 127.67, 125.82, 124.99, 35.14, 32.78, 31.07, 23.01, 20.43, 20.02, 13.32. Appearance: white solid. Melting point: 191-194° C. UPLC/MS: 1-100% ACN(+0.01% FA) in 5 mins; r.t.=3.15 mins; ES(+): 201.3.

### Compound XL: Synthesis of sodium 2-(5-(2,2-dimethylcyclopropyl)pentyl)cyclopropanecarboxylate

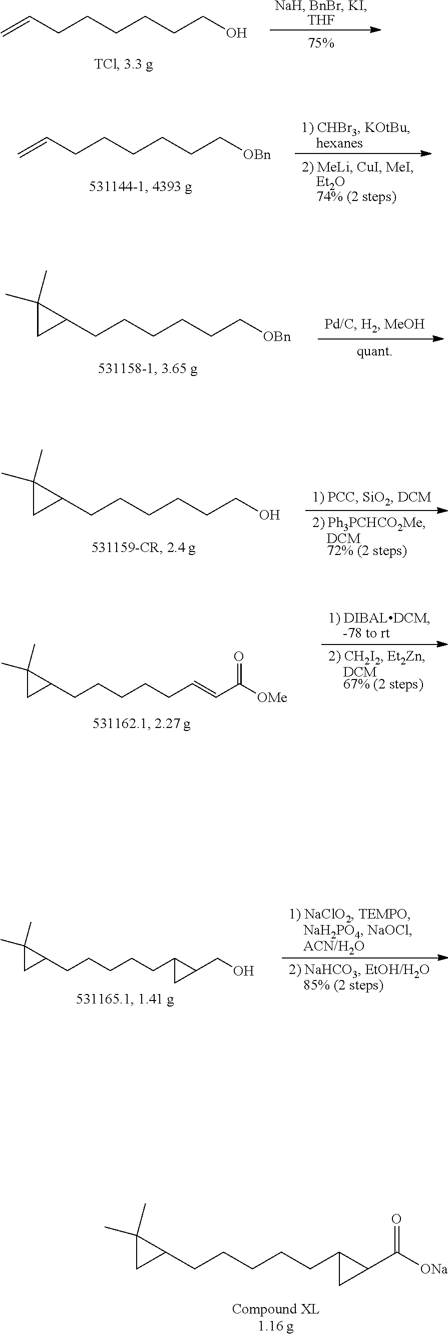

Step 1: ((oct-7-enyloxy)methyl)benzene. oct-7-en-1-ol (3.30 g) was converted to ((oct-7-enyloxy)methyl)benzene in a manner similar to previous examples (see, e.g., Compound I, step 3) to give 4.93 g of desired product.

Step 2: ((6-(2,2-dibromocyclopropyl)hexyloxy)methyl)benzene. ((oct-7-enyloxy)methyl)benzene(x g) was converted to ((6-(2,2-dibromocyclopropyl)hexyloxy)-methyl)benzene in a manner similar to previous examples (see, e.g., Compound II, step 1) to give 8.70 g of desired product.

Step 3: ((6-(2,2-dimethylcyclopropyl)hexyloxy)methyl)benzene. ((6-(2,2-dibromocyclopropyl)hexyloxy)methyl)benzene (8.70 g) was converted to ((6-(2,2-dimethylcyclopropyl)hexyloxy)methyl)benzene in a manner similar to previous examples (see, e.g., Compound III, step 1) to give 3.65 g of desired product.

Step 4: 6-(2,2-dimethylcyclopropyl)hexan-1-al. ((6-(2,2-dimethylcyclopropyl)hexyloxy)methyl)benzene (3.65 g) was converted to 6-(2,2-dimethylcyclopropyl)hexan-1-ol in a manner similar to previous examples (see, e.g., Compound I, step 5) to give 2.40 g of desired product.

Step 5: 6-(2,2-dimethylcyclopropyl)hexanal. 6-(2,2-dimethylcyclopropyl)hexan-1-ol (2.40 g) was converted to 6-(2,2-dimethylcyclopropyl)hexanal in a manner similar to previous examples (see, e.g., Compound IX, step 2) to give 2.30 g of desired product.

Step 6: (E)-methyl 8-(2,2-dimethylcyclopropyl)oct-2-enoate. 6-(2,2-dimethylcyclopropyl)hexanal (2.30 g) was converted to (E)-methyl 8-(2,2-dimethylcyclopropyl)oct-2-enoate in a manner similar to previous examples (see, e.g., Compound IX, step 3) to give 2.27 g of desired product.

Step 7: (E)-8-(2,2-dimethylcyclopropyl)oct-2-en-1-ol. (E)-methyl 8-(2,2-dimethylcyclopropyl)oct-2-enoate (2.27 g) was converted to (E)-8-(2,2-dimethylcyclopropyl)oct-2-en-1-ol in a manner similar to previous examples (see, e.g., Compound I, step 2) to give 1.96 g of desired product.

Step 8: (2-(5-(2,2-dimethylcyclopropyl)pentyl)cyclopropyl)methanol. (E)-8-(2,2-dimethylcyclopropyl)oct-2-en-1-ol (1.96 g) was converted to (2-(5-(2,2-dimethylcyclopropyl)-pentyl)cyclopropyl)methanol in a manner similar to previous examples (see, e.g., Compound VI, step 5) to give 1.41 g of desired product.

Step 9: 2-(5-(2,2-dimethylcyclopropyl)pentyl)cyclopropanecarboxylic acid. (2-(5-(2,2-dimethylcyclopropyl)-pentyl)cyclopropyl)methanol (1.41 g) was converted to 2-(5-(2,2-dimethylcyclopropyl)pentyl)cyclopropanecarboxylic acid in a manner similar to previous examples (see, e.g., Compound I, step 6) to give 1.25 g of desired product.

Step 10: Sodium 2-(5-(2,2-dimethylcyclopropyl)pentyl)cyclopropanecarboxylate. 2-(5-(2,2-dimethylcyclopropyl)pentyl)cyclopropanecarboxylic acid (1.25 g) was converted to sodium 2-(5-(2,2-dimethylcyclopropyl)pentyl)cyclopropanecarboxylate in a manner similar to previous examples (see, e.g., Compound I, step 7) to give 1.15 g of the final product. ¹H NMR (400 MHz, Methanol-d4) δ 1.47-1.22 (m, 11H), 1.18 (td, J=6.2, 5.7, 2.3 Hz, 2H), 1.01 (d, J=4.8 Hz, 6H), 0.95 (ddd, J=9.6, 5.1, 3.7 Hz, 1H), 0.52-0.39 (m, 2H), 0.35 (dd, J=8.5, 4.0 Hz, 1H), −0.08-−0.20 (m, 1H). ¹³C NMR (101 MHz, Methanol-d4) δ 182.11, 33.31, 29.94, 29.42, 29.14, 29.06, 26.60, 24.60, 23.00, 20.61, 19.04, 18.86, 14.76, 13.36. Appearance: white solid. Melting point: 180-183° C.

### Compound XLI: Synthesis of sodium 3-hexyl-2,2-dimethylcyclopropanecarboxylate

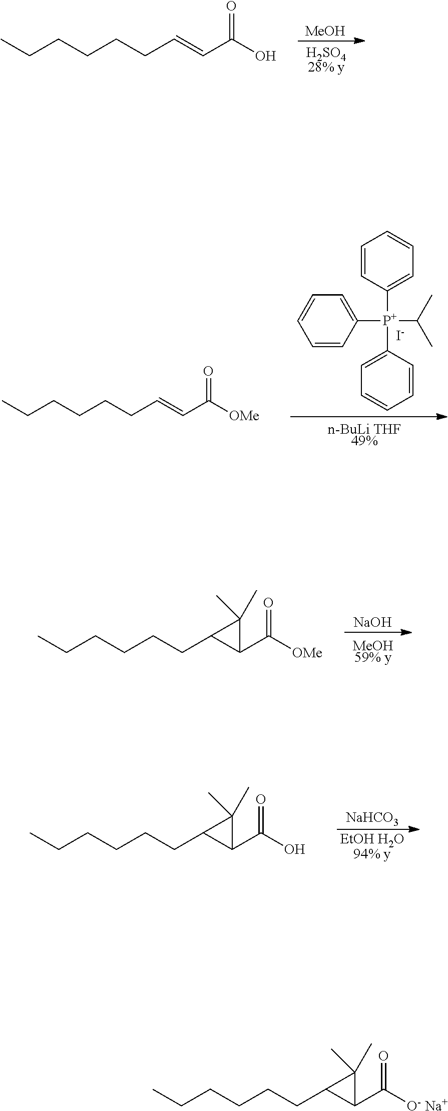

Step 1: (E)-methylnon-2-enoate. This compound was prepared as for Compound I, Step 1, to give a colorless oil (25.12, 92% y). ¹H NMR (400 MHz, Chloroform-d) δ 7.0-6.9 (m, 1H), 5.85-5.77 (m, 1H), 3.72 (s, 3H), 2.24-2.14 (m, 2H), 1.50-1.38 (m, 2H), 1.36-1.20 (m, 6H), 0.86 (t, J=6.6 Hz, 3H).

Step 2: Methyl 3-hexyl-2,2-dimethylcyclopropanecarboxylate. To a suspension of Isopropyl phosphonium iodide (100 g, 1.75 eq) in THF (1 L), at room temperature and under Argon atmosphere, was added n-BuLi 2.5M in hexanes in 20 min. The mixture was stirred for 30 min and then a solution of (E)-methyl non-2-enoate (22.5 g, 1 eq) in THF (25 ml) was added dropwise in 10 min. The reaction was stirred at ambient temperature for 2 h and heated at 40° C. for 1 h. The reaction was quenched with HCl 2N (25 ml) and diluted with water (400 ml) and hexanes (400 ml); a white solid appeared. The solid was filtered and discarded (triphenylphosphine). The filtrate was concentrated, and the residue was purified on silica gel with 0 to 1.5% ethyl acetate in hexane to afford a colorless oil (13.84 g, 49%). ¹H NMR (400 MHz, Chloroform-d) δ 3.62 (s, 3H), 1.2-1.12 (m, 10H), 1.1 (s, 3H), 1.09-1.05 (m, 2H), 1.05 (s, 3H), 0.86 (t, J=6.6 Hz, 3H).

Step 3: 3-hexyl-2,2-dimethylcyclopropanecarboxylic acid. A solution of methyl 3-hexyl-2,2-dimethylcyclopropanecarboxylate (13.84 g, 1 eq) in methanol (300 ml) was treated with a solution of sodium hydroxide (13.04 g, 5.0 eq) in water 150 ml); and the reaction was stirred vigorously at 40° C. for 4 days. The reaction mixture was diluted with water (500 ml) and washed with TBME (3×100 ml). The reaction was then acidified with 2M aqueous hydrochloric acid (100 ml) and extracted with TBME (3×100 ml). The organic extract was washed with saturated aqueous sodium chloride solution (50 ml); dried over sodium sulfate; filtered and evaporated in vacuo to give a colorless oil (7.69 g, 59% y). ¹H NMR (400 MHz, Chloroform-d) δ 1.4-1.22 (m, 11H), 1.22 (s, 3H), 1.15-1.12 (m, 1H), 1.15 (s, 3H), 0.85 (t, J=6.6 Hz, 3H).

Step 4: Sodium 3-hexyl-2,2-dimethylcyclopropanecarboxylate. This compound was prepared as for Compound I, Step 7 to give a white solid (1.18 g, 94% y). ¹H NMR (400 MHz, Methanol-d₄) δ 1.42-1.22 (m, 10H), 1.18 (s, 3H), 1.17-1.1 (q, J=5.8 Hz, 1H), 1.10 (s, 3H), 1.05-1.04 (d, J=5.47 Hz, 1H), 0.89 (t, J=7.03 Hz, 3H). ¹³C NMR (101 MHz, Methanol-d₄) δ 180.32, 37.08, 31.69, 31.22, 29.58, 28.90, 28.39, 23.88, 22.30, 20.66, 20.57, 13.02. MP: >278° C.

### Compounds XLII and XLIII: Synthesis of sodium (Z)-2-(2-pentylcyclobutylidene)acetate and 2-(2-Pentylcyclobutyl)acetate

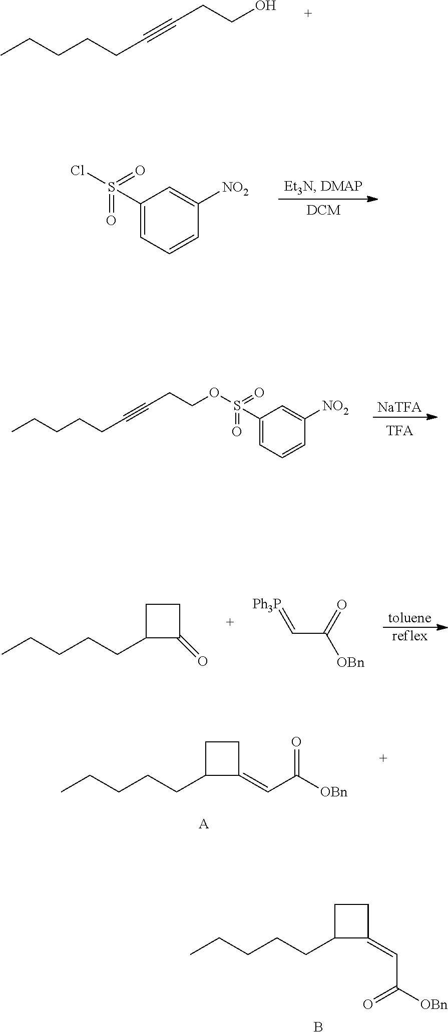

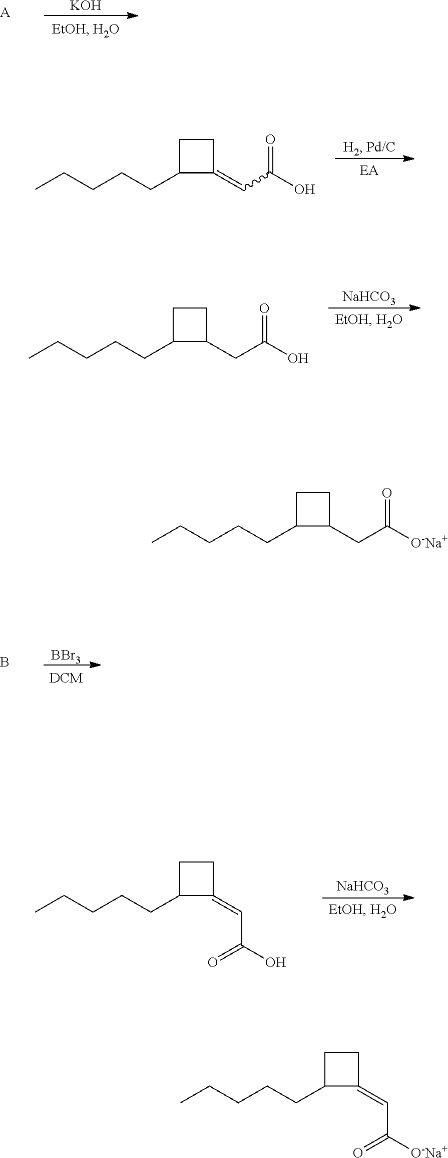

Step 1: non-3-ynyl 3-nitrobenzenesulfonate. To a solution of non-3-yn-1-ol (2.26 mL) in DCM (100 mL) at 0° C. were added Et₃N (2.2 mL, 1.1 eq.), DMAP (2 mg, cat.) and 3-nitrobenzene-1-sulfonyl chloride (3.16 g, 1 eq.). Reaction was stirred at rt for 18 hours. 1N HCl was added and org. phase was separated, washed with brine, dried over Na₂SO₄, filtered and concentrated. Residue was purified on silica gel (0-30% EA/hexanes) to afford desired sulfonate (3.28 g, 71%) as a colorless oil (see Angewandte Chemie, International Edition, 46(24), 4527-4529; 2007).

Step 2: 2-pentylcyclobutanone. To a solution non-3-ynyl 3-nitrobenzenesulfonate (3.28 g) in TFA (15 mL) was added NaTFA. Reaction was stirred at 50° C. for 4 days. Once at rt, reaction was poured in NaHCO₃ and MTBE was added. Org. phase was separated, washed with brine, dried over Na₂SO₄, filtered and concentrated. Residue was purified on silica gel (0-4% EA/hex) to afford desired cyclobutanone (672 mg, 48%) as a colorless oil (see Tet. Let. 32, 3847, 1966).

Step 3: (E)-benzyl 2-(2-pentylcyclobutylidene)acetate & (Z)-benzyl 2-(2-pentylcyclobutylidene)acetate. To a solution of 2-pentylcyclobutanone (670 mg) in toluene (24 mL) was added benzyl (triphenylphosphoranylidene)acetate (3.92 g, 2 eq.). Reaction was stirred at reflux for 18 hours. Once at rt, reaction was concentrated and residue was purified on silica gel (0-3% EA/hex) to afford desired E-alkene (116 mg, 9%) as a colorless oil and desired Z-alkene (223 mg, 17%) as a colorless oil (see Yvonne Lear, U. Ottawa, thesis entitled “The regiospecific synthesis and reactivity of 2-hydroxybenzocyclobutenones” 1997, doi: 10.20381/ruor-13853, http://hdl.handle.net/10393/4430).

Step 4: 2-(2-pentylcyclobutylidene)acetic acid, cis/trans mixture. To a solution of (E)-benzyl 2-(2-pentylcyclobutylidene)acetate (116 mg) in EtOH (4 mL) were added KOH (119 mg, 5 eq.) and water (0.4 mL). Reaction was stirred at reflux for 3 hours. Once at rt, reaction was concentrated, water and 1N HCl were added until pH 2 was reached. MTBE was added and org. phase was separated, washed with brine, dried over Na₂SO₄, filtered and concentrated. Residue was purified on silica gel (0-50% EA/hexanes) to afford a mixture of E and Z isomers of acid (30 mg, 26%) as a white solid.

Step 5: 2-(2-pentylcyclobutyl)acetic acid, cis/trans mixture. To a N₂ bubbled solution of 2-(2-pentylcyclobutylidene)acetic acid, cis/trans mixture (81 mg) in ethyl acetate (5 mL) was added Pd/C 10% w/w (47 mg, 0.1 eq.). N₂ was removed and H₂ was bubbled in the reaction for 5 min. And then, reaction was stirred under H₂ atmosphere for 18 hours. H₂ was removed and N₂ was bubbled. Celite™ was added and reaction was filtered on Celite™. Filtrate was concentrated to afford desired mixture of acid diastereoisomers (66 mg, 81%) as a colorless oil.

Step 6: Sodium 2-(2-Pentylcyclobutyl)acetate (compound XLIII), cis/trans mixture. This compound was prepared as for Compound I, Step 7 to give a white solid. ¹H NMR (400 MHz, Methanol-d₄) δ 2.81-1.84 (m, 6H), 1.73-1.47 (m, 2H), 1.47-1.09 (m, 8H), 0.96-0.82 (m, 3H). ¹³C NMR (101 MHz, Methanol-d₄) δ 181.09, 180.55, 44.71, 42.53, 39.91, 38.52, 37.56, 35.95, 35.05, 31.84, 31.83, 30.01, 26.90, 26.64, 24.72, 24.55, 24.30, 24.25, 22.35, 22.33, 13.01. ESI-MS m/z 185.08 (M+1).

Step 1B: (Z)-2-(2-pentylcyclobutylidene)acetic acid. To a solution of (Z)-benzyl 2-(2-pentylcyclobutylidene)acetate (133 mg) in DCM (5 mL) at −78° C. was added dropwise BBr₃ 1M/DCM (0.98 mL, 2 eq.). Reaction was warmed to 0° C. and stirred for 4 hours at 0° C. Reaction was quenched with aq. sat. NaHCO₃ then 1N HCl was added to reach pH 2. MTBE was added and org. phase was separated, washed with brine, dried over Na₂SO₄, filtered and concentrated. Residue was purified on silica gel (0-50% EA/hexanes) to afford desired acid (28 mg, 31%) as a yellow oil (see JACS, 127(22), 7994, 2005).

Step 2B: Sodium (Z)-2-(2-pentylcyclobutylidene)acetate (compound XLII). This compound was prepared as for Compound I, Step 7 to give an off-white solid (50 mg, quant.). ¹H NMR (400 MHz, Methanol-d₄) δ 5.59 (q, J=2.4 Hz, 1H), 3.10-2.85 (m, 3H), 2.13 (dtd, J=10.7, 9.3, 5.3 Hz, 1H), 1.67-1.55 (m, 2H), 1.47-1.38 (m, 1H), 1.41-1.21 (m, 6H), 0.98-0.85 (m, 3H). ¹³C NMR (101 MHz, Methanol-d₄) δ 174.94, 162.11, 116.99, 44.42, 33.73, 31.65, 29.52, 26.40, 23.87, 22.28, 12.98. ESI-MS m/z 183.18 (M+1). MP: 268-273° C.

### Compound XLIV: Synthesis of sodium 3-(3-hexyl-2,2-dimethylcyclopropyl) propanoate

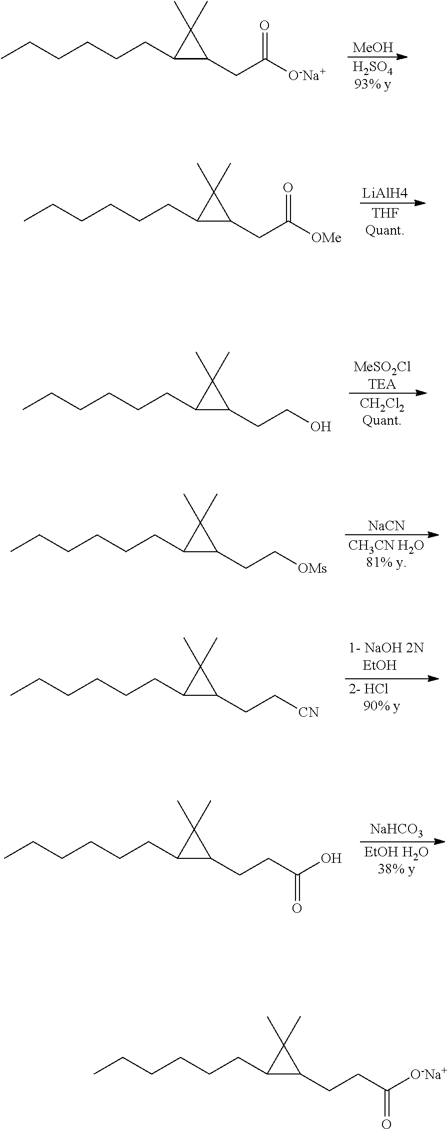

Step 1: Methyl 2-(3-hexyl-2,2-dimethylcyclopropyl)acetate. A solution of sodium 2-(3-hexyl-2,2-dimethylcyclopropyl) acetate (2.28 g, 1 eq) in methanol (200 ml) was treated with sulfuric acid (2 ml) and the reaction is stirred at ambient temperature for 22 h. Methanol was evaporated in vacuo, and the residue is dissolved in TBME (300 ml). The solution was washed with water (3×50 ml) and with saturated aqueous sodium chloride (50 ml); dried over sodium sulfate; filtered and evaporated in vacuo to give a colorless oil (2.05 g, 93% y). ¹H NMR (400 MHz, Chloroform-d) δ 3.65 (s, 3H), 2.32-2.29 (dd, J=7.42 Hz, 2H), 1.4-1.24 (m, 10H), 1.04-1.01 (d, J=9.77 Hz, 6H), 0.92-0.88 (t, J=7.03 Hz, 3H), 0.48-0.43 (m, 1H), 0.27-0.22 (m, 1H).

Step 2: 2-(3-hexyl-2,2-dimethylcyclopropyl)ethanol. A solution of methyl 2-(3-hexyl-2,2-dimethylcyclopropyl) acetate (2.05 g, 1 eq) in THF (60 ml) was added in 1.5 h to a suspension of Lithium Aluminum Hydride (344 mg, 1.0 eq) in THF (200 ml) at 0° C. The mixture was stirred at ambient temperature for 1.5 h. The reaction was quenched at 0° C. with Ethyl acetate (50 ml) and a saturated solution of ammonium chloride (50 ml). The mixture was filtered; the filtrate was concentrated in vacuo. The residue was dissolved in EtOAc (100 ml). This solution was washed with saturated aqueous sodium chloride solution (15 ml); dried over sodium sulfate; filtered and evaporated in vacuo to give a colorless oil (1.80 g, quant. y). ¹H NMR (400 MHz, Methanol-d₄) δ 3.6-3.5 (t, J=7.02 Hz, 2H), 1.65-1.55 (m, 1H), 1.5-1.4 (m, 1H), 1.4-1.2 (m, 10H), 1.04-1.01 (d, J=9.77 Hz, 6H), 0.91-0.87 (t, J=7.02 Hz, 3H), 0.21-0.11 (m, 2H).

Step 3: 2-(3-hexyl-2,2-dimethylcyclopropyl)ethyl methanesulfonate. The 2-(hexyl-2,2-dimethylcyclopropyl) ethanol (1.80 g, 1 eq) was dissolved in dry methylene chloride (50 ml). The triethylamine (1.10 g, 1.2 eq) was added, followed by methane sulfonyl chloride (1.25 g, 1.2 eq). The mixture was stirred at ambient temperature for 22 h and then diluted with water (50 ml) and methylene chloride (50 ml). The organic phase was separated and washed with saturated aqueous sodium chloride solution (35 ml); dried over sodium sulfate; filtered and evaporated in vacuo to give a colorless oil (2.62 g, quant. y). ¹H NMR (400 MHz, Methanol-d₄) δ 4.24-4.21 (t, J=6.64 Hz, 2H), 3.04 (s, 3H), 1.82-1.76 (m, 1H), 1.72-1.65 (m, 1H), 1.40-1.29 (m, 10H), 1.05-1.03 (d, J=3.91 Hz, 6H), 0.91-0.88 (t, J=4.30 Hz, 3H), 0.25-0.20 (m, 2H).

Step 4: 3-(3-hexyl-2,2-dimethylcyclopropyl)propanenitrile. A solution of 2-(3-hexyl-2,2-dimethylcyclopropyl) ethyl methane sulfonate (2.62 g, 1 eq) in acetonitrile (100 ml) is added in 2-3 min with stirring to a solution of Sodium cyanide (2.21 g, 5.0 eq). The mixture was then placed in a preheated bath at 100° C. and heated to reflux for 24 h. The reaction was cooled and poured in a mixture of water and TBME (150 ml/150 ml). The organic phase was separated and washed with saturated aqueous sodium chloride solution (100 ml); dried over sodium sulfate/charcoal; filtered on Fiberglass and evaporated in vacuo to give a yellow oil (1.51 g, 81% y). ¹H NMR (400 MHz, Methanol-d₄) δ 2.47-2.43 (t, J=7.03 Hz, 2H), 1.77-1.67 (m, 1H), 1.63-1.54 (m, 1H), 1.41-1.26 (m, 10H), 1.05-1.04 (d, J=51.2 Hz, 6H), 0.97-0.88 (t, J=6.65 Hz, 3H), 0.27-0.22 (m, 2H).

Step 5: 3-(3-hexyl-2,2-dimethylcyclopropyl) propanoic acid. The 3-(3-hexyl-2,2-dimethylcyclopropyl) propanenitrile is dissolved in NaOH 2N (18.2 ml) and Ethanol 95% (20 ml) and refluxed for 22 h. The mixture was diluted with water (30 ml) and washed with TBME (30 ml). The aqueous phase was acidified with HCl 2N and the compound extracted with TBME (3×20 ml). The organic phase was washed with saturated aqueous sodium chloride solution (30 ml); dried over sodium sulfate; filtered and evaporated in vacuo to give an orange oil. (1.68 g). The oil was dissolved in acetone (50 ml) and t-Butylamine (520 mg, 1 eq) was added; the mixture was heated at 50° C. and then cooled to −5° C. to afford the T-Butyl amine salt as a white solid. The solid was filtered, washed with cold acetone and dried. To regenerate the free acid, the salt was dissolved in H₃PO₄ 10% (40 ml) and TBME (40 ml). The organic phase was separated and washed with saturated aqueous sodium chloride solution (30 ml); dried over sodium sulfate; filtered and evaporated in vacuo to give a yellow oil (1.49 g, 90% y). ¹H NMR (400 MHz, Methanol-d₄) δ 2.33-2.30 (t, J=7.43 Hz, 2H), 1.69-1.62 (m, 1H), 1.58-1.51 (m, 1H), 1.38-1.24 (m, 10H), 1.03-1.01 (d, J=8.20 Hz, 6H), 0.92-0.88 (t, J=6.65 Hz, 3H), 0.18-0.14 (m, 2H).

Step 6: Sodium 3-(3-hexyl-2,2-dimethylcyclopropyl) propanoate. A solution of 3-(3-hexyl-2,2-dimethylcyclopropyl)propanoic acid (1.46 g, 1 eq) in ethanol (100 ml) was treated with a solution of sodium bicarbonate (541.8 mg, 1 eq) in water (20 ml); and the reaction was stirred at ambient temperature for 2 h. The solution was then concentrated to a small volume in vacuo; diluted with water to 100 ml/g; filtered (0.2 μm; PES); and lyophilized to give the desired sodium salt as a white solid (600 mg, 38% y). ¹H NMR (400 MHz, Methanol-d₄) δ 2.22-2.17 (t, J=8.20 Hz, 2H), 1.68-1.60 (m, 1H) 1.58-1.49 (m, 1H), 1.38-1.25 (m, 10H), 1.03-1.00 (d, J=12.29 Hz, 6H), 0.91-0.88 (t, J=6.64 Hz, 3H), 0.17-0.10 (m, 2H). ¹³C NMR (101 MHz, Methanol-d₄) δ 181.38, 38.27, 31.71, 30.81, 30.75, 30.02, 29.20, 29.03, 26.46, 22.34, 20.93, 20.90, 18.86, 13.05. MP: >220° C.

### Compound XLV: Synthesis of sodium (E)-3-(3-hexyl-2,2-dimethylcyclopropyl)acrylate

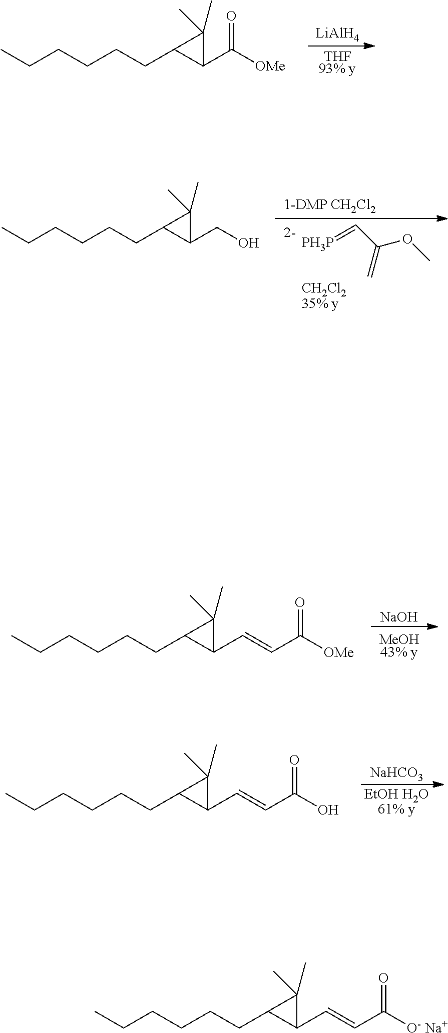

Step 1: (3-hexyl-2,2-dimethylcyclopropyl)methanol. A solution of methyl 3-hexyl-2,2-dimethylcyclopropanecarboxylate (3.11 g, 1 eq) in THF (20 ml) was added in 1 h to a suspension of Lithium Aluminum Hydride (833.8 mg, 1.5 eq) in THF (60 ml) at 0° C. The mixture was heated at 70° C. for 2 h, and cooled at ambient temperature and stirred for 18 h. The reaction was quenched with Ethyl acetate (6 ml) and a saturated solution of ammonium chloride. The mixture was filtered; the filtrate was concentrated in vacuo. The residue was dissolved in TBME (100 ml). This solution was washed with saturated aqueous sodium chloride solution (15 ml); dried over sodium sulfate; filtered and evaporated in vacuo to give a colorless oil (2.52 g, 93% y). ¹H NMR (400 MHz, Methanol-d₄) δ 3.68-3.64 (dd, J=11.3 Hz, 1H), 3.41-3.36 (dd, J=11.3 Hz, 1H), 1.37-1.29 (m, 10H), 1.05-1.01 (d, J=6.3 Hz, 6H), 0.91 (t, J=6.6 Hz, 3H), 0.49-0.44 (m, 1H), 0.32-0.29 (m, 1H).

Step 2: (E)-methyl 3-(3-hexyl-2,2-dimethylcyclopropyl)acrylate. The Dess Martin Periodinane (7.59 g, 3.0 eq) was added portion wise at 0° C. in 5 minutes to a solution of (3-hexyl-2,2-dimethylcyclopropyl) methanol (1.10 g, 1 eq) in methylene chloride (60 ml). The mixture was stirred at ambient temperature for 2 h. The mixture was diluted with methylene chloride (60 ml), quenched with a 1/1 saturated solution of sodium Carbonate and sodium thiosulfate and stirred for 30 min. The compound was extracted with methylene chloride (3×40 ml). The organic extract was washed with saturated aqueous sodium chloride solution (50 ml); dried over sodium sulfate; filtered and concentrated in vacuo to half of his volume. To this solution was added (carbomethoxymethylene) triphenyl phosphorane (2.39 g, 1.2 eq). The mixture was stirred at ambient temperature overnight (22 h). The solvent was evaporated in vacuo and the residue was purified on silica gel with 0 to 5% Ethyl acetate in hexanes to yield a yellowish oil (490 mg, 35% y). ¹H NMR (400 MHz, Methanol-d₄) δ 6.75-6.54 (dd, J=11.3 Hz, 1H), 5.85-5.80 (d, J=11.3 Hz, 1H), 3.65 (s, 3H), 1.5-1.10 (m, 10H), 1.05-1.01 (d, J=6.3 Hz, 6H), 1.01-0.91 (m, 2H), 0.91 (t, J=6.6 Hz, 3H).

Step 3: (E)-3-(3-hexyl-2,2-dimethylcyclopropyl)acrylic acid. A solution of (E)-methyl 3-(3-hexyl-2,2-dimethylcyclopropyl)acrylate (13.84 g, 1 eq) in methanol (40 ml) was treated with a solution of sodium hydroxide (411 mg, 5.0 eq) in water 10 ml); and the reaction was stirred at ambient temperature for 20 h. The reaction mixture was concentrated in vacuo, the residue was acidified with 2M aqueous hydrochloric acid (40 ml) and extracted with TBME (3×20 ml). The organic extract was washed with saturated aqueous sodium chloride solution (10 ml); dried over sodium sulfate; filtered and evaporated in vacuo to give a crude oil. This oil was purified on silica gel with 0 to 20% ethyl acetate in hexanes to afford a clear yellow oil (199.7 g, 43% y). ¹H NMR (400 MHz, Methanol-d₄) δ 6.75-6.68 (dd, J=11.3 Hz, 1H), 5.82-5.78 (d, J=11.3 Hz, 1H), 1.49-1.29 (m, 10H), 1.05-1.01 (d, J=6.3 Hz, 6H), 1.06-0.87 (m, 5H).

Step 4: Sodium (E)-3-(3-hexyl-2,2-dimethylcyclopropyl)acrylate. This compound was prepared as for Compound I, Step 7, to give a white solid (124.4 mg, 61% y). ¹H NMR (400 MHz, Methanol-d₄) δ 6.45-6.39 (dd, J=15.2 Hz, 1H), 5.85-5.82 (d, J=15.6 Hz, 1H), 1.46-1.29 (m, 10H), 1.11-1.09 (d, J=6.3 Hz, 6H), 1.02-0.99 (dd, J=5.08 Hz, 1H), 0.91-0.88 (t, J=6.6 Hz, 3H), 0.764-0.751 (m, 1H). ¹³C NMR (101 MHz, Methanol-d₄) δ174.37, 145.11, 125.55, 34.66, 34.27, 31.64, 29.49, 28.79, 28.61, 24.49, 22.29, 21.89, 20.22, 13.01. MP: >220° C.

### Example 2: Effects of Representative Compounds Disclosed Herein on the Induction of Hemoglobin Production In Vitro

The effects of representative compounds disclosed herein on the induction of hemoglobin production in human bone marrow chronic myelogenous leukemia cells (K562) was assessed using the 2,7-diaminofluorene (DAF) assay, which measures the oxidization of DAF by the pseudoperoxidase activity of free hemoglobin. K562 cells were incubated for 5 days with the various compounds (Compounds 1, 11 and Ill) at the noted concentrations. On day 5, cells were centrifuged and washed in PBS. 2×10⁶ cells were lysed in 140 μl of NP-40 (0.01%, 5 minutes on ice). 2 mg of DAF (2,7-diaminofluorene, 97%, Aldrich, cat #D17106-1G) was resuspended in 200 μl of Glacial Acetic Acid 90% and a working solution was prepared as followed: 100 μl of DAF+100 μl of H₂O₂ 30%+10 ml Tris-HCl 0.1M/6M Urea pH 7, Vortex. 50 μl of cell lysate was transferred to a well and 150 μl of DAF working solution was added, followed by incubation for 8 min in the dark and assessment of optical density (OD) at 610 nm. The results are depicted in Table 3.

TABLE 3
Hemoglobin quantification (O.D. by DAF
method) for compounds I, II, III

| | Hb | |
|---|---|---|
| Groups | (fold increase vs. Control) | t-test |
| Control | 1 | |
| Compound I 500 μM | 0.81 | ns |
| Compound II 125 μM | 1.2 | 0.01 |
| Compound III 250 μM | 1.25 | 0.016 |

### Example 3: Effect of a Representative Compounds Disclosed Herein on the In Vivo Induction of Immune Cell Proliferation or Chemoprotection

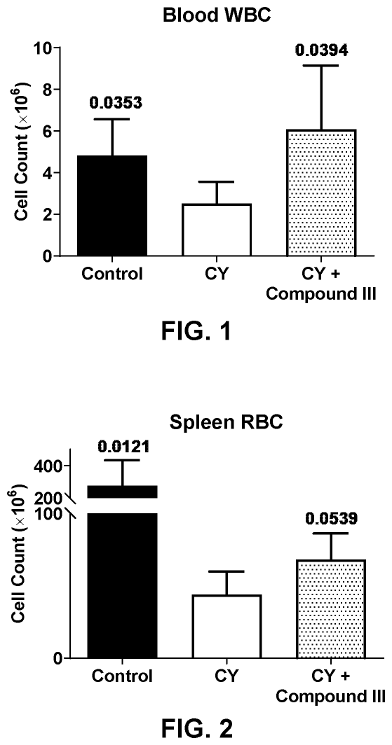

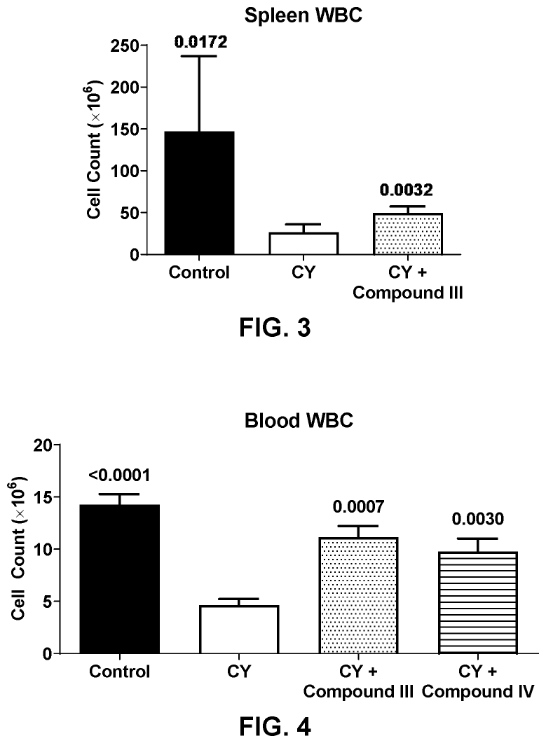

Female C57BL/6 mice, 6- to 8-weeks old, were immunosuppressed by treatment with 100 mg/kg of cyclophosphamide administered intravenously at day 0. To examine the immunoprotective effect of compound III, mice were pre-treated orally at day −3, −2 and −1 at day 0 with the compound. Mice were sacrificed at day +5 by cardiac puncture and cervical dislocation. Then, a gross pathological observation of the femurs (as a source of bone marrow cells) was recorded. After sacrifice, tissues were crushed in phosphate buffered saline (PBS) and cells were counted with a hemacytometer. A significant increase in white blood cell count (FIG. 1) as well as in the spleen white (FIG. 2) and red (FIG. 3) cells was observed after oral pre-treatment with compound III in cyclophosphamide-treated mice. Furthermore, some treated animals with oral pre-treatment with compound III showed an increase in the spleen white (FIG. 2) and red (FIG. 3) cell count relative to non-immunosuppressed animals (control).

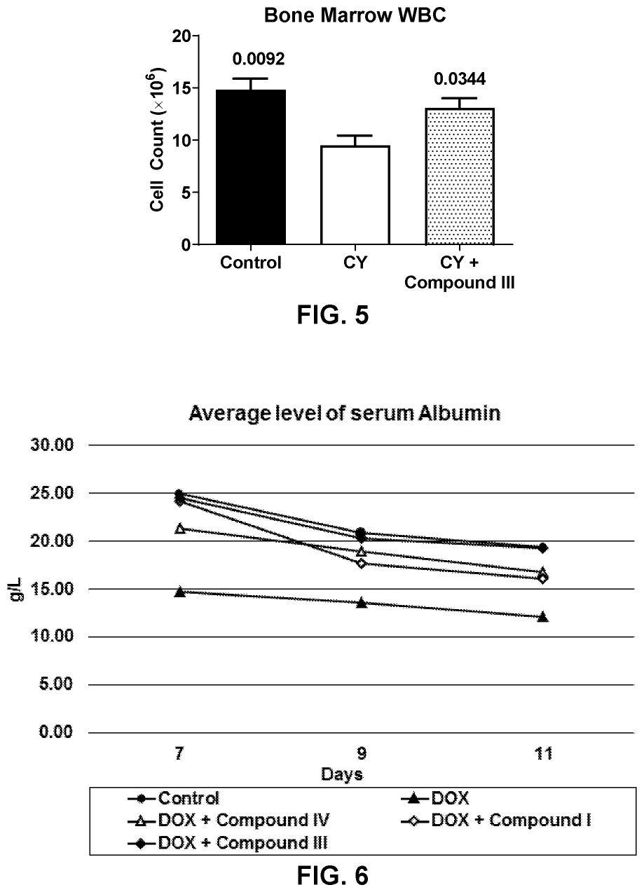

In vivo induction of immune cell proliferation or chemoprotection by using 100 mg/kg of compound III or compound IV was also undertaken. Compound III increases blood and bone marrow white cells (FIGS. 4 and 5) and compound IV increases white blood cells (FIG. 5).

### Example 4: In Vivo Effect of Representative Compounds Disclosed Herein on Renal Protection in Doxorubicin-Induced Nephrotoxicity Model

Demonstration of the in vivo protection by oral administration of representative compounds disclosed herein was undertaken in the doxorubicin-induced nephrotoxicity model using the following procedure. C57BL/6 mice (6 to 10-weeks old) were treated with compounds prophylactically from day −3 to day 10. Nephrotoxicity was induced by an intravenous injection of 10 mg/kg of doxorubicin at day 0. Serum albumin was monitored at day 11.

As shown in FIG. 6, prophylactic treatment with Compounds I, III and IV inhibit the decrease of serum albumin induced by doxorubicin. Decrease of serum albumin correlates with the kidney lesions induced by doxorubicin. The above provides in vivo evidence that the compounds described herein may be useful for preventing and/or treating drug-induced (doxorubicin) apoptosis, inflammation and subsequent fibrosis-related organ dysfunction, notably of the kidney.

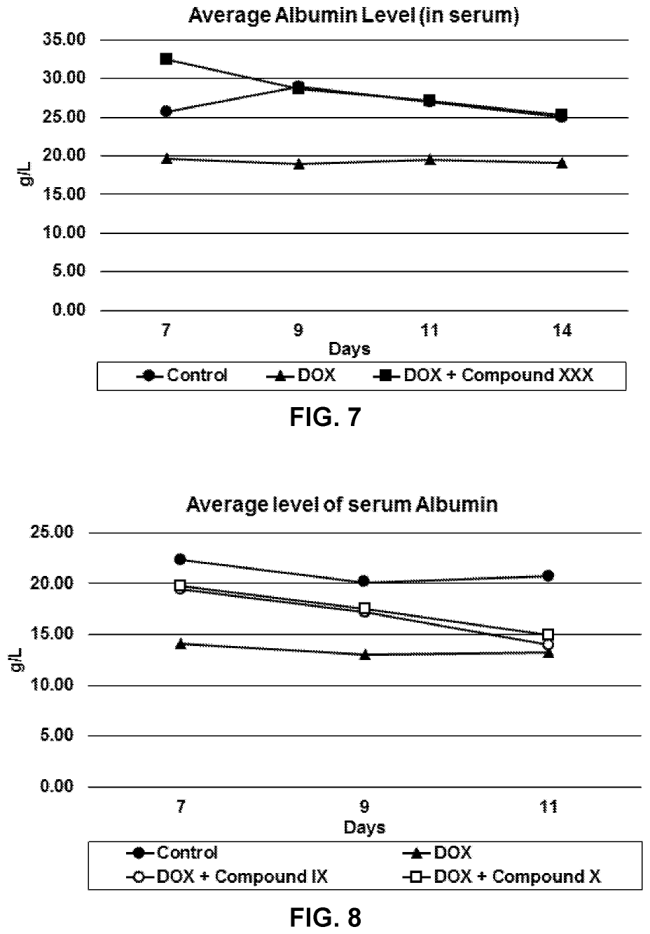

As shown in FIGS. 7 and 8, prophylactic treatment with Compounds XXX, IX or X inhibits the decrease of serum albumin induced by doxorubicin, proving evidence that these compounds prevent doxorubicin-induced lesions, damage-inducing glomerulosclerosis, tubular dilatation and ultimately fibrosis.

### Example 5: Effect of Compound III on Renal Protection in an Adenine-Induced Chronic Kidney Disease (CKD) Model

Adenine-supplementation is an effective tool to study the onset and progression of fibrosis and CKD-associated sequelae. Six- to eight-weeks old male C57BL/6 mice were fed a regular (CTRL, n=9) or custom diet consisting of regular chow supplemented with 0.25% adenine for 30 days. After 7 days, mice were administered vehicle (H₂O, n=9) or Compound II (100 mg/kg, n=10) by daily oral gavage. Blood sampling was done at day 0, 7 and 30. Reticulocytes were quantified by flow cytometry analysis. Serum urea and creatinine levels were measured at endpoint by ELISA and HPLC respectively. Renal histology was assessed using H&E and Masson's trichrome stained kidney sections.

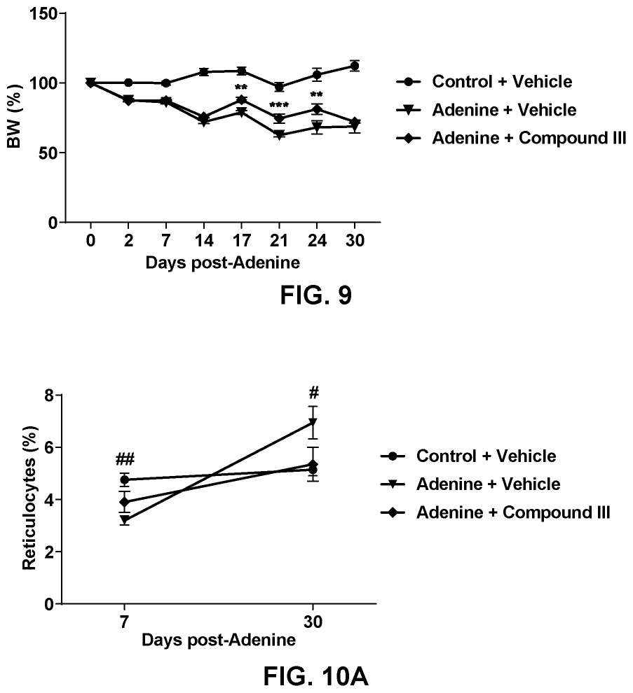

Adenine decreased bodyweight, which was significantly improved by Compound III at day 17, 21 and 24 (FIG. 9).

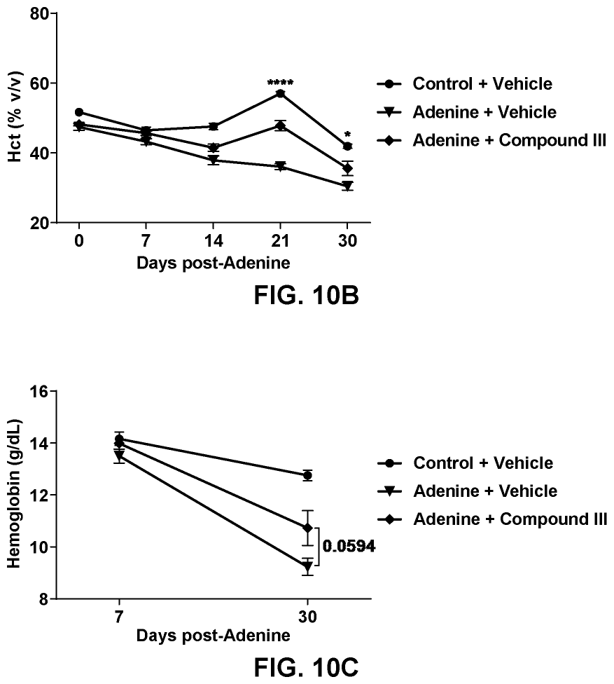

Anemia was apparent as hematocrit (Hct) began to decline as early as 7 days post-adenine, however this was significantly improved by Compound III at day 14, 21 and 30 (FIG. 10B). Flow cytometry analyses revealed reduced reticulocyte counts in vehicle-treated adenine mice relative to CTRL mice at day 14, however at day 30, levels were increased. Compound III treatment maintained reticulocyte counts to normal levels (FIG. 10A). Similarly, hemoglobin was decreased in Ad-fed mice, but levels in Compound III mice trended higher (p=0.059) (FIG. 10C).

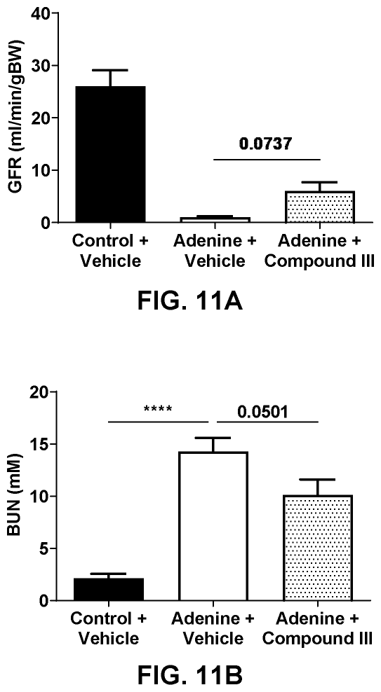

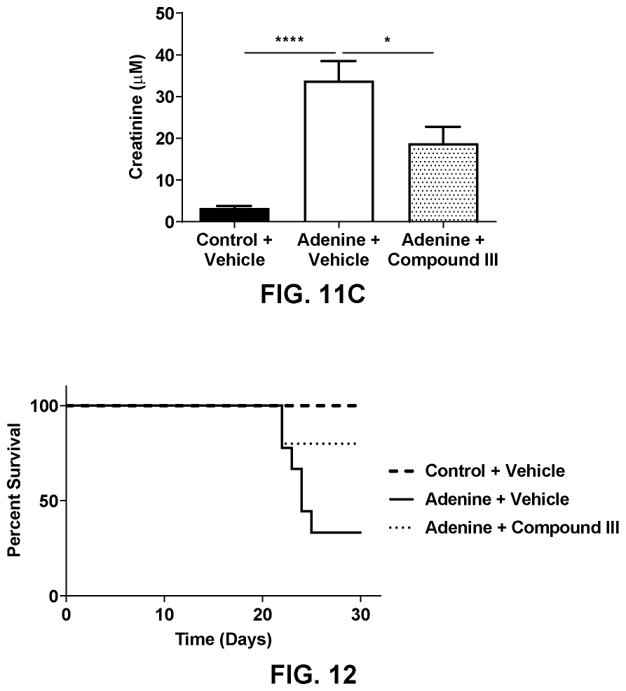

At endpoint, blood urea nitrogen and serum creatinine were increased by Ad-feeding, however treatment with Compound III led to a reduction of these levels (FIG. 11B, C). Survival rate increased from 30% in the vehicle-treated group to 80% in Compound III-treated mice (FIG. 12).

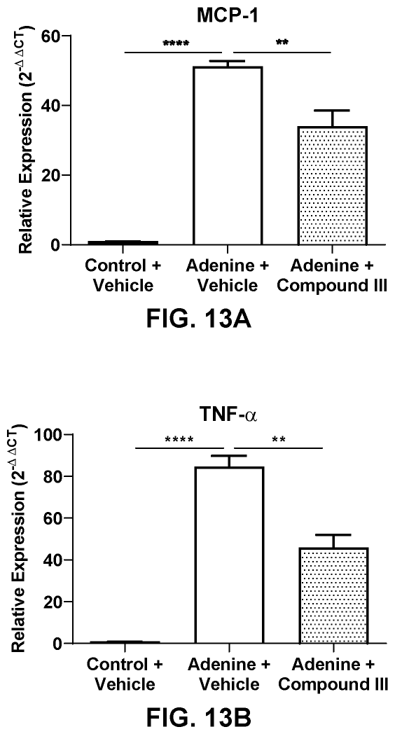

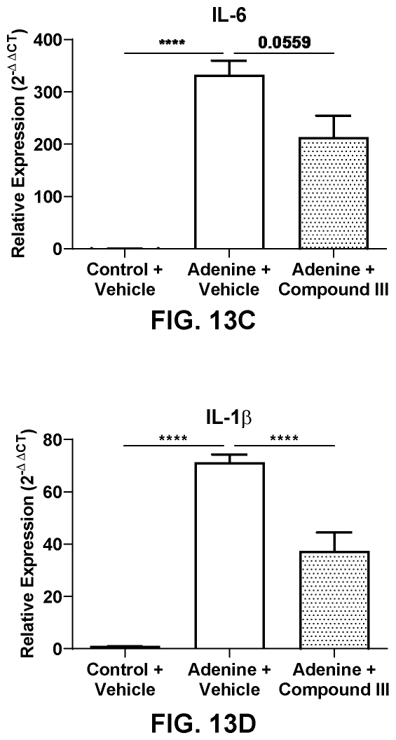

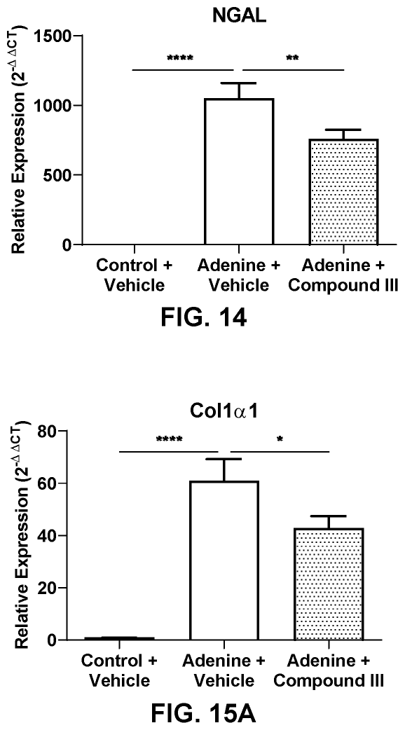

As shown in FIGS. 13A-D, pro-inflammatory gene expression was significantly reduced in kidney treated with Compound III. Also, the level of expression of a biomarker of kidney injury, neutrophil gelatinase-associated lipocalin (NGAL), was reduced by treatment with Compound III (FIG. 14). The association between NGAL overexpression and a variety of clinical situations leading to AKI (cardiac surgery, kidney transplantation, contrast nephropathy, haemolytic uraemic syndrome and in the intensive care setting) or to CKD (lupus nephritis, glomerulonephritides, obstruction, dysplasia, polycystic kidney disease, IgA nephropathy) is well known.

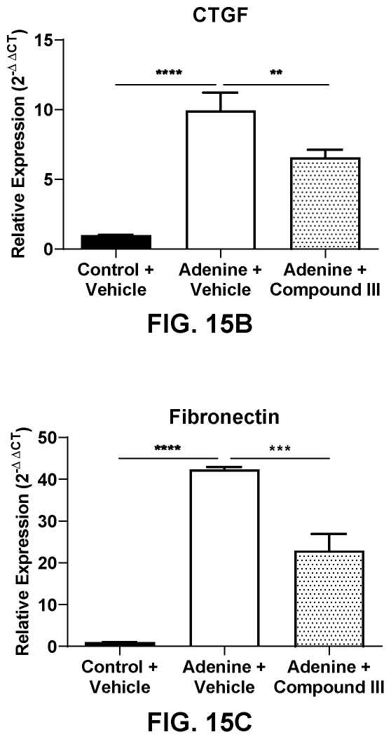

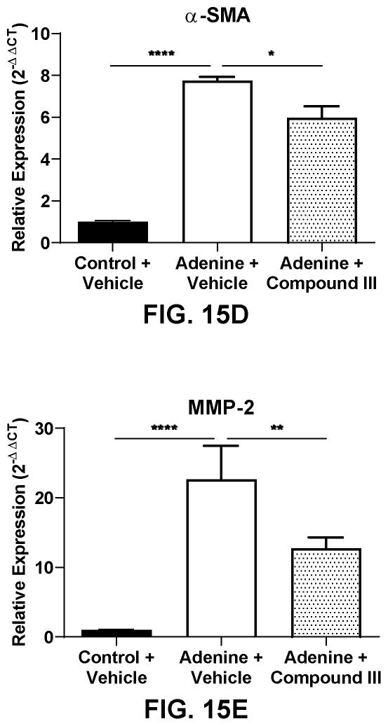

As shown in FIGS. 15A-E, expression of several pro-fibrotic genes including collagen 1a1, CTGF, fibronectin, α-SMA and MMP2 was decreased by Compound III treatment.

Taken together, these results show that Compound III improves several key renal functional and structural abnormalities as well as pro-inflammatory and pro-fibrotic markers in adenine-induced CKD including anemia, fibrosis and renal function decline leading to improved survival rates.

### Example 6: In Vivo Effect of Compound III on Kidney Protection in 516 Nephrectomy Model

Demonstration of the in vivo protection effect of Compound III on renal tissue was also undertaken in the 5/6 nephrectomized (Nx) rat model using the following procedure. Male 6 weeks-old Sprague Dawley rats were subjected to 5/6 nephrectomy or sham operations. Under fluothane anesthesia, renal ablation was achieved by removing two-thirds of the left kidney followed by a right unilateral nephrectomy 7 days later. Sham rats underwent exposition of the kidneys and removal of the perirenal fat. Twenty-one days after the first operation, rats were randomized in the study by their reduced glomerular filtration rate (GFR) of creatinine indicating a dysfunction of the kidney. Animals that underwent the sham operation were given vehicle (saline) and were used as controls. Nx animals were divided in groups receiving the vehicle or Compound I. Saline or Compound I was given by gastric gavage once daily up to the sacrifice. GFR was measured every three weeks in order to assess the severity of this end-stage renal disease model. Rats were sacrificed at day 150.

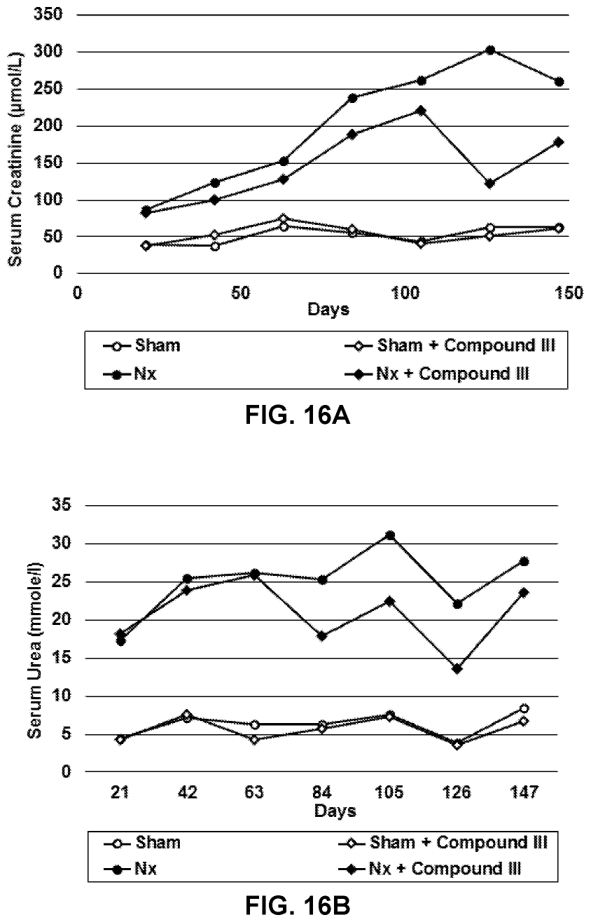

FIGS. 16A and 168 depict the level of serum creatinine and urea, respectively, in Nx and Compound III-treated Nx rats relative to sham animals. Compound III was shown to reduce the level of serum creatinine and urea, indicating an improvement in kidney function.

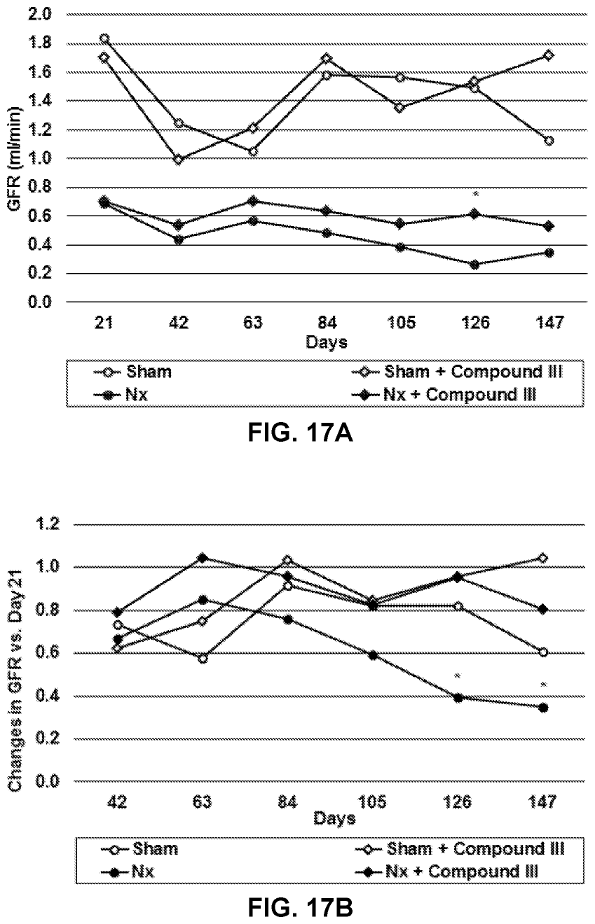

FIGS. 17A and 17B represent the improvement of the GFR in Nx and Compound III-treated Nx rats over treatment period relative to the initial GFR (before treatment) at day 21. A significant improvement of GFR was observed in Compound III-treated Nx rats relative to a 50% deterioration of GFR in Nx rats (control).

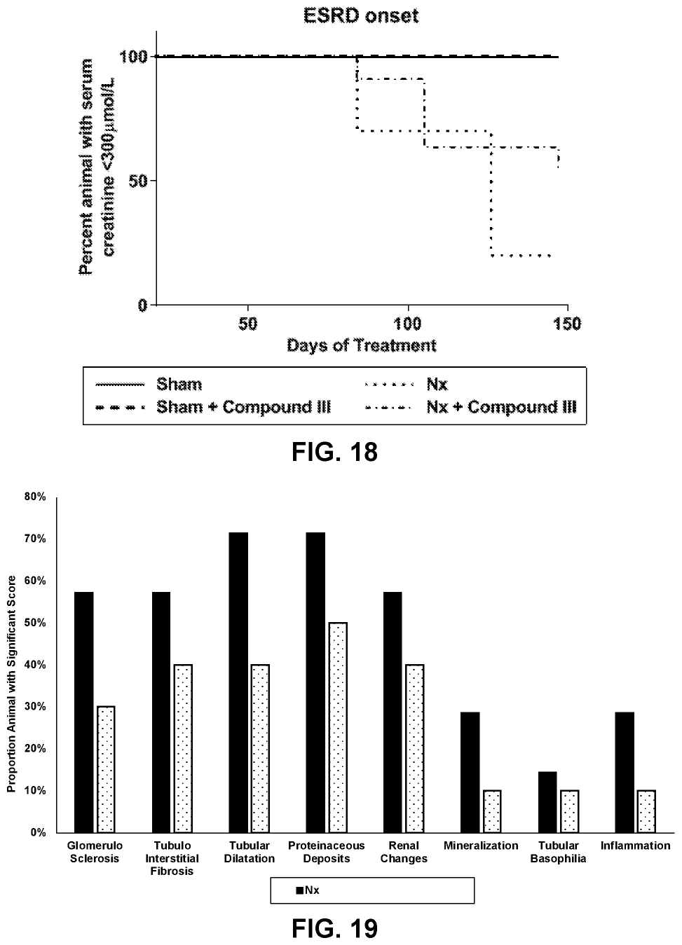

FIG. 18 depicts the percentage of animals having serum creatinine levels greater than 300 μmol/L, which is indicative of renal failure or end-stage renal disease (ERSD), and shows that the proportion of animals reaching this threshold is reduced in the Compound III-treated Nx group.

FIG. 19 shows the beneficial effect of compound III at the histological level. Compound III reduces the glomerulosclerosis, tubulointerstitial fibrosis, tubular dilatation, proteinaceous deposits, renal changes, mineralization, tubular basophilia and kidney inflammation.

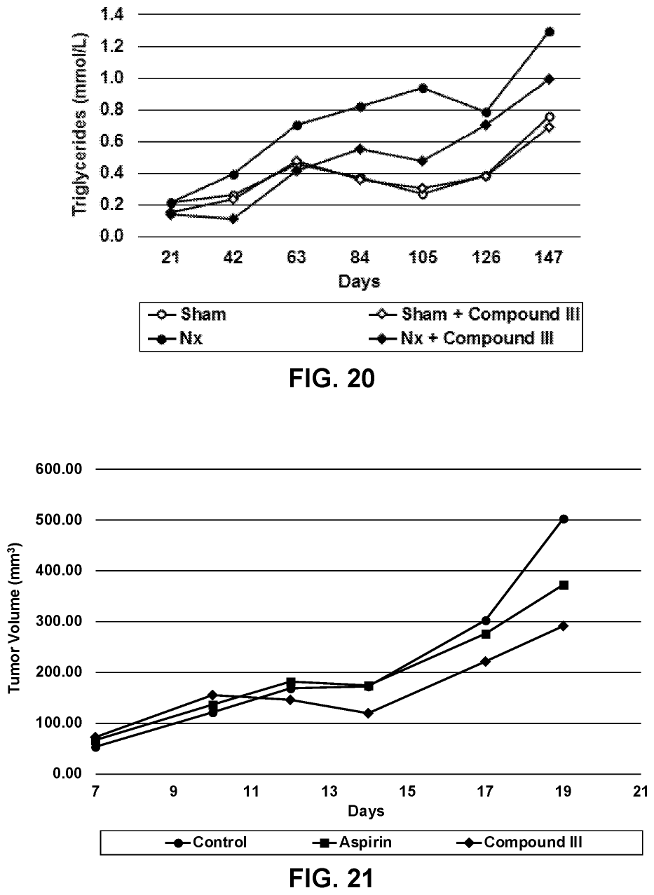

It was noted that the level of serum triglycerides increases more significantly over time in the 5/6 NX rats relative to the sham group. FIG. 20 shows that Compound III significantly reduces the levels of serum triglycerides in 5/6 Nx rats, which indicates a metabolic effect through regulation of triglyceride levels and a better liver function.

### Example 7: Antitumor Effect of Compound III on a Primary P815 Mastocytoma Tumor

The syngeneic tumor P815 is a DBA/2 (H-2^{d})-derived mastocytoma obtained from ATCC (TIB64). P815 cells were grown in DMEM containing 10% fetal bovine serum. At day 0, 50 μL of 5×10⁵ viable P815 cells were intradermally injected to produce localized tumors in 6- to 8-weeks old DBA/2 mice. Animals were then serially monitored by manual palpation for evidence of tumor. Mice were then treated every day with oral administration of vehicle (negative control), acetylsalicylic acid (ASA) (positive control, 50 mg/kg) or Compound III (100 mg/kg). Mice were sacrificed around day 23 (depending on the experiment). Serial tumor volume was obtained by bi-dimensional diameter measurements with calipers, using the formula 0.4 (a×b²) where “a” was the major tumor diameter and “b” the minor perpendicular diameter. Tumors were palpable, in general, 3-5 days post-inoculation.

FIG. 21 shows the effect of oral administration of Compound III and the gold standard compound acetylsalicylic acid (ASA, positive control) on primary tumor P815 cells. Compound III administration led to a significant reduction (p<0.05) of P815 (mastocytoma) tumor growth relative to control.

### Example 8: Anti-Fibrotic Effect of Compound III

Collagen and α-SMA (alpha-Smooth Muscle Actin) are well-known markers of fibrosis. The effect of several compounds of Formula I was assessed on i) expression of collagen mRNA in HK-2 cells (an immortalized proximal tubule epithelial cell line from normal adult human kidney) induced by the pro-fibrotic cytokine TGF-β; and ii) expression of α-SMA mRNA in NRK-49F cells (an immortalized normal rat kidney fibroblasts cell line) induced by TGF-β. HK-2 cells (ATCC #CRL-2190) were cultured at 70,000 cells/well in DMEM/F-12+10% FBS for 24 h. Cells were starved overnight in DMEM/F-12+0.2% FBS and then treated with the compounds and TGF-β1 (8 ng/ml) for 24 h. RNA was isolated with TRIzol® reagent and expression of collagen, more specifically collagen of type 1 expressed by the gene COL1A1, was determined by quantitative real-time PCR. qPCR analysis of relative gene expression was performed with TaqMan® Gene Expression assays using the ΔΔCt method. mRNA expression levels were normalized against GAPDH endogenous control levels in each sample and calculated relative to control TGFβ1-treated cells. NRK-49F cells (ATCC #CRL-1570) were cultured at 50,000 cells/well in DMEM/F-12+5% FBS for 24 h. Cells were starved overnight in DMEM/F-12+0.5% FBS and then treated with compounds and TGF-β1 (3 ng/ml) for 24 h. RNA was isolated with TRIzol® reagent and expression of α-SMA (ACTA2 gene) was determined by quantitative real-time PCR. qPCR analysis of relative gene expression was performed with TaqMan® Gene Expression assays using the ΔΔCt method. mRNA expression levels were normalized against GAPDH endogenous control levels in each sample and calculated relative to control TGFβ1-treated cells. Results of these experiments are depicted in Table 4.

TABLE 4
Effect of compounds I-XLV on the expression of collagen (COL1A1) and α-
SMA mRNA induced by TGF-β

| | | α-SMA | COL1A1 |
|---|---|---|---|
| Compound | Structure of compound or salt thereof | inhibition | inhibition |
| I | 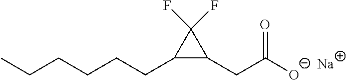 | ++++ | ++++ |
| II | 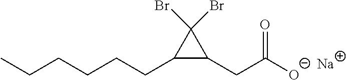 | ++++ | NT |
| III | 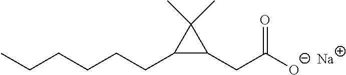 | ++++ | +++ |
| IV | 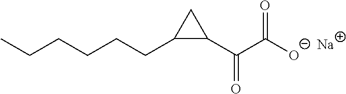 | ++++ | +++ |
| V | 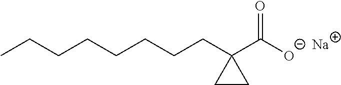 | +++++ | − |
| VI | 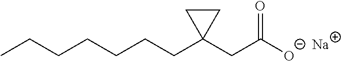 | ++++ | + |
| VII | 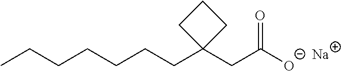 | +++ | +++ |
| VIII | 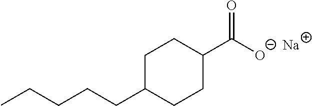 | NT | NT |
| IX | 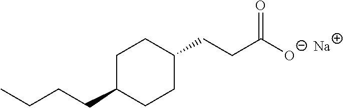 | ++++ | ++++ |
| X | 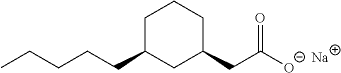 | ++++ | − |
| XI | 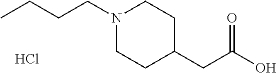 | − | NT |
| XII | 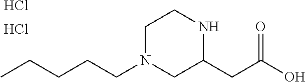 | + | NT |
| XIII | 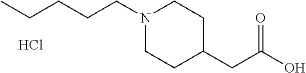 | + | NT |
| XIV | 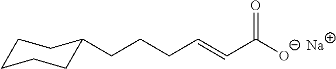 [SMILES: O=C([O-])/C=C/CCCC1CCCCC1 ; [Na+]] | ++++ | ++++ |
| XV | 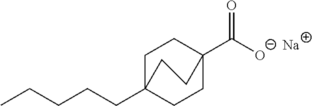 [SMILES: CCCCCC12CCC(C(=O)[O-])(CC1)CC2 ; [Na+]] | ++ | ++ |
| XVI | 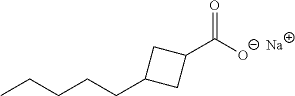 | ++ | − |
| XVII | 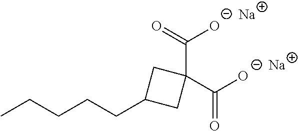 | ++++ | − |
| XVIII | 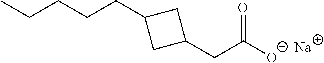 | ++++ | ++++ |
| XIX | 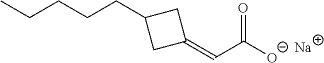 | ++++ | ++++ |
| XX | 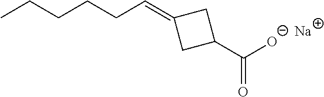 | ++++ | ++++ |
| XXI | 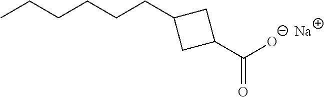 | ++++ | ++++ |
| XXII | 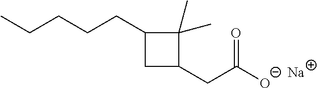 | +++ | +++ |
| XXIII | 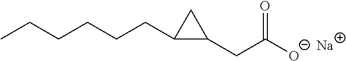 | ++++ | ++ |
| XXIV | 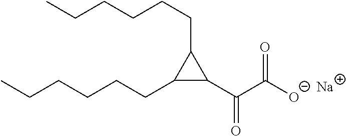 [SMILES: CCCCCCC1C(CCCCCC)C1C(=O)C(=O)[O-] ; [Na+]] | + | NT |
| XXV | 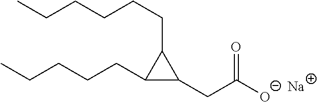 [SMILES: CCCCCCC1C(CCCCC)C1CC(=O)[O-] ; [Na+]] | − | NT |
| XXVI | 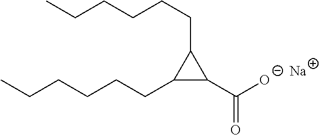 [SMILES: CCCCCCC1C(CCCCCC)C1C(=O)O ; [Na+]] | + | NT |
| XXVII | 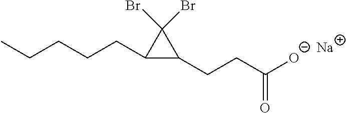 | ++++ | ++++ |
| XXVIII | 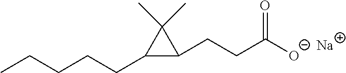 | ++++ | + |
| XXIX | 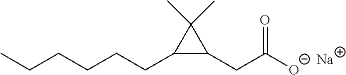 | +++ | +++ |
| XXX | 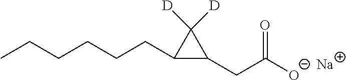 | +++ | ++++ |
| XXXI | 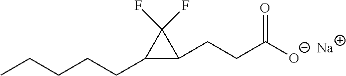 | ++++ | ++++ |
| XXXII | 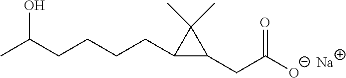 [SMILES: CC(O)CCCCC1C(CC(=O)[O-])C1(C)C ; [Na+]] | − | − |
| XXXIII | 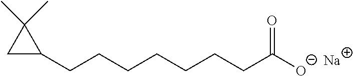 | ++++ | ++++ |
| XXXIV | 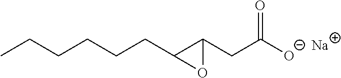 | ++++ | +++ |
| XXXV | 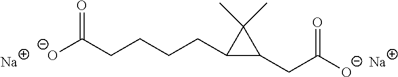 [SMILES: [Na+] ; CC1(C)C(CCCCC(=O)[O-])C1CC(=O)[O-] ; [Na+]] | ++++ | − |
| XXXVI | 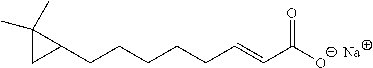 | ++++ | + |
| XXXVII | 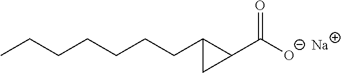 | ++++ | ++++ |
| XXXVIII | 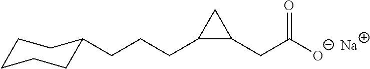 [SMILES: O=C([O-])CC1CC1CCCC1CCCCC1 ; [Na+]] | +++ | − |
| XXXIX | 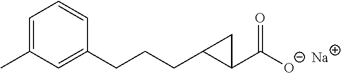 [SMILES: Cc1cccc(CCCC2CC2C(=O)[O-])c1 ; [Na+]] | +++ | + |
| XL | 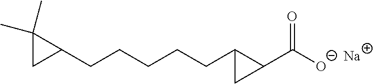 [SMILES: CC1(C)CC1CCCCCC1CC1C(=O)[O-] ; [Na+]] | ++++ | +++ |
| XLI | 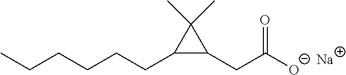 | ++++ | ++++ |
| XLII | 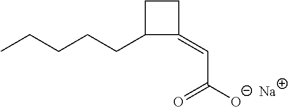 | ++++ | ++++ |
| XLIII | 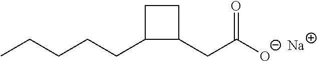 | ++++ | ++++ |
| XLIV | 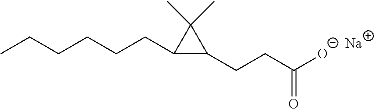 [SMILES: CCCCCCC1C(CCC(=O)[O-])C1(C)C ; [Na+]] | − | ++++ |
| XLV | 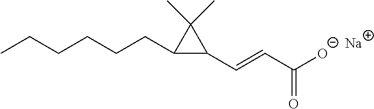 [SMILES: CCCCCCC1C(/C=C/C(=O)[O-])C1(C)C ; [Na+]] | ++ | + |

++++: 75-100% inhibition
+++: 50-74% inhibition
++: 25-49% inhibition
+: 1-24% inhibition
−: No detectable effect
NT: not tested

### Example 9: Antihypertensive Activity of Compound III

Antihypertensive activity was tested in a model of DKD/CKD induced by adenine supplementation and streptozotocin, the latter inducing death of pancreatic beta-cells and mimicking type 1 diabetes. Adenine-supplementation is a suitable model to study the onset and progression of fibrosis and CKD-associated sequelae. Lewis female rats (125 g) received 60 mg/kg of streptozotocin at day 0. On day 2, blood glucose and body weights were taken. Animals presenting a glucose level over 250 mg/dl and a weight loss were considered diabetics and were randomized. At day 21, adenine supplementation (600 mg/kg) was started to induce kidney lesions. Treatment with compound III started at day 21 at a dose of 100 mg/kg. Blood pressure measurement was performed on anesthetized (isoflurane 2%) Lewis female rat approximately one hour after oral administration of Compound III using the CODA system.

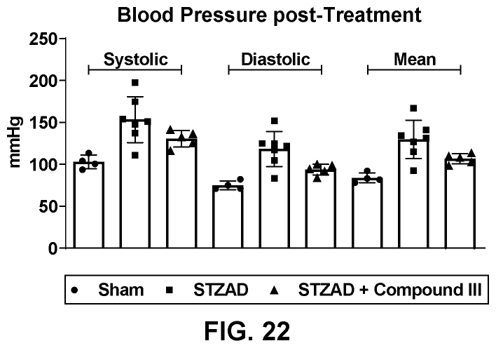

As shown in FIG. 22, Compound III reduces both systolic and diastolic blood pressure in compromised diabetic rats.

### Example 10: Signaling Properties of Representative Compounds Disclosed Herein on the Fatty Acid GPR40, GPR84 and GPR120 Receptors

It was next assessed whether representative compounds disclosed herein could modulate the activity of receptors responsive to free fatty acids (FFAs). GPR40 and GPR120 are activated by both medium- and long-chain FFAs, while GPR84 is exclusively responsive to medium-chain FFAs. Binding of FFAs to GPR40 on pancreatic β-cells leads to activation of several signaling pathways involved in insulin secretion and targeting this receptor has shown to be a promising new treatment for type 2 diabetes (T2DM), and a dual GPR40 and GPR120 agonist showed potent activity on both adipose tissue lipolysis and glucose metabolism, highlighting the potential of these receptors in FA and glucose metabolism (Satapati, S. et al. J. Lipid. Res. 58, 1561-1578). GPR84 is expressed in monocytes, neutrophils and macrophages and is induced under pro-inflammatory stimuli, and has been shown to be involved in metabolic dysregulation, e.g., in obesity-related metabolic syndrome (Simard et al., Scientific Reports volume 10, Article number 12778 (2020)).

Methods

Plasmids: The cDNA clones for human GPR40 and GPR84 receptors, human β-arrestin 2, Gαᵢ₂, Gβ₁, and Gγ₂ were obtained from the cDNA Resource Center (www.cdna.org). A plasmid encoding the human GPR120-L (long isoform) cDNA was obtained from R&D Systems. GPR120-S (short isoform) was generated by replacing the BglII-BsgI fragment from GPR120-L by a gBlock gene fragment (Integrated DNA Technologies, IA) lacking the DNA sequence corresponding to the extra 16 amino acids found in the third intracellular loop of the long form. GFP10 (F64L, S147P, S202F and H231L variant of Aequorea victoria GFP) gBlocks gene fragments (Integrated DNA Technologies) and linker were inserted in frame at the N-terminus of human Gγ₂, or at the C-terminus of GPR40 and GPR120. Rluc8 (A55T, C124A, S130A, K136R, A143M, M185V, M253L, and S287L variant of the Renilla luciferase) gBlocks gene fragment (Integrated DNA Technologies) was inserted with linkers in between residues 91 and 92 of Gα₂ or at the N-terminus of β-arrestin 2.

BRET measurements: a Gαᵢ bioluminescence resonance energy transfer (BRET) biosensor was used to directly monitor GPR84-mediated activation of Gαᵢ. The Gαᵢ biosensor consists of a Rluc8-tagged Gαᵢ₂ subunit, a GFP10-tagged Gγ₂ subunit, and an untagged Gβ₁. Agonist stimulation and ensuing GPR84 activation triggers a physical separation between the RLuc8-Gαᵢ donor and the GFP10-Gγ₂ acceptor, resulting in a decrease in BRET signal whose amplitude is correlated to ligand efficacy. A BRET-based assay that allows the monitoring of Rluc8-tagged β-arrestin 2 recruitment to GFP10-tagged GPR40 or GFP10-tagged GPR120 was used to assess GPR40 or GPR120 activation. Transiently transfected HEK293 cells were seeded in 96-well white clear bottom Costar microplates (Fisher Scientific) coated with poly-D-lysine (Sigma-Aldrich) and left in culture for 24 hours. Cells were washed once with Tyrode's buffer (140 mmol/L NaCl, 1 mmol/L CaCl₂, 2.7 mmol/L KCl, 0.49 mmol/L MgCl₂, 0.37 mmol/L NaH₂PO₄, 5.6 mmol/L glucose, 12 mmol/L NaHCO₃, and 25 mmol/L HEPES, pH 7.5) and the Rluc8 substrate coelenterazine 400A (Prolume, Lakeside, Ariz.) added at a final concentration of 5 μmol/L in Tyrode's buffer. Ligands were incubated with cells at room temperature for 5 minutes (G protein) or 25 minutes (β-arrestin) before reading BRET signal. In GPR84 antagonist mode, cells were treated with 125 μmol/L of the GPR84 agonist sodium decanoate in combination with test compounds. BRET readings were collected using an Infinite M1000 microplate reader (Tecan, Morrisville, N.C.). BRET² readings between Rluc8 and GFP10 were collected by sequential integration of the signals detected in the 370 to 450 nm (Rluc8) and 510 to 540 nm (GFP10) windows. The BRET signal was calculated as the ratio of light emitted by acceptor (GFP10) over the light emitted by donor (Rluc8). The values were corrected to net BRET by subtracting the background BRET signal obtained in cells transfected with Rluc8 constructs alone. Ligand-promoted net BRET values were calculated by subtracting vehicle-induced net BRET from ligand-induced net BRET.

Results

The results are reported in Table 5.

TABLE 5
Activity of the compounds on GPR84, GPR40 and GPR120 signaling

| | GPR84 | GPR40 | GPR120 |
|---|---|---|---|
| | (antagonist mode) | (agonist mode) | (agonist mode) |
| Compounds | IC50 Gαi (μM) | EC50 β-arr2 (μM) | EC50 β-arr2 (μM) |
| 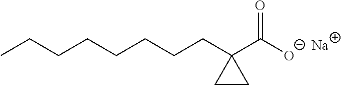 | 115 | 164 | 245 |
| 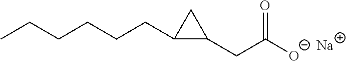 | agonist EC50 <16 | 170 | 272 |
| 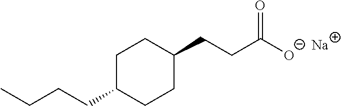 | 22 | 21 | 22 |
| 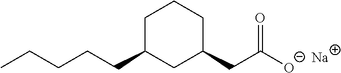 | 56 | 12 | 377 |
| 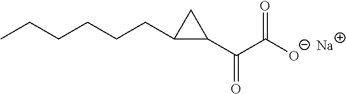 | agonist EC50 36.9 | 321 | >500 |
| 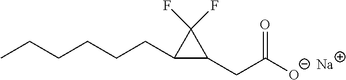 | 117 | 126 | 383 |
| 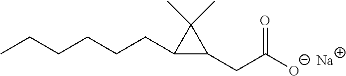 | 56 | 30 | >125 |
| 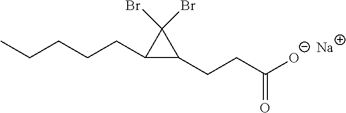 | 20 | 16 | 45 |
| 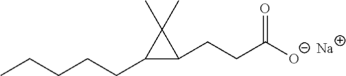 | >63 | 27 | >63 |
| 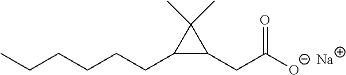 | | >63 | >63 |
| 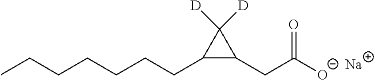 [SMILES: [2H]C1([2H])C(CCCCCCC)C1CC(=O)[O-] ; [Na+]] | Agonist EC50 23 | 149 | 299 |
| 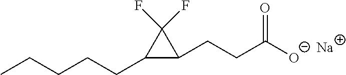 | Agonist EC50 79 | 133 | >500 |
| 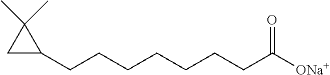 | Agonist EC50 5 | 11 | 77 |
| 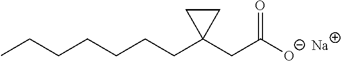 | >500 | 122 | 500 |
| 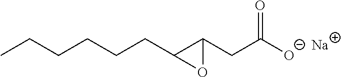 | Agonist EC50 114 | >500 | >500 |
| 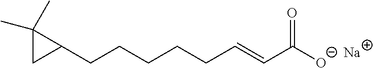 | Agonist EC50 6 | 22 | 61 |
| 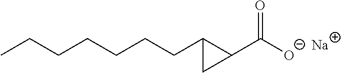 | Agonist EC50 13 | 49 | >250 |
| 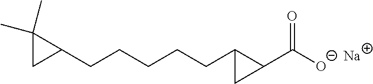 [SMILES: CC1(C)CC1CCCCCC1CC1C(=O)[O-] ; [Na+]] | 63 | 14.3 | 35 |
| 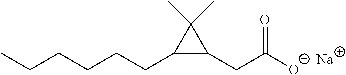 | 468 | 174 | >500 |
| 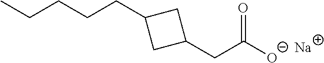 | Agonist EC50 35 | 87 | >500 |
| 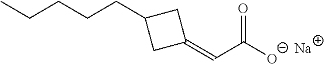 | Agonist EC50 <16 | 70 | 88 |
| 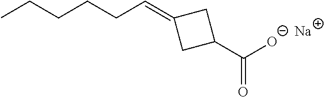 | 131 | 252 | >500 |
| 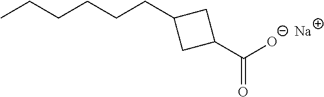 | Agonist EC50 73 | 63.4 | 382 |
| 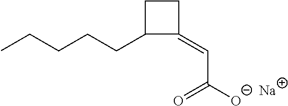 | 180 | 211 | 307 |
| 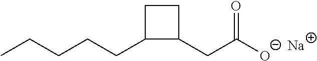 | 173 | 83 | >500 |
| 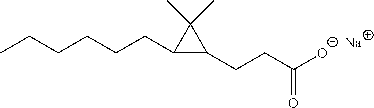 [SMILES: CCCCCCC1C(CCC(=O)[O-])C1(C)C ; [Na+]] | 9 | 10 | >31 |
| 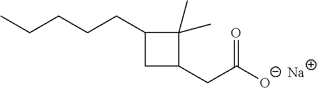 | 49 | 15 | 90 |
| 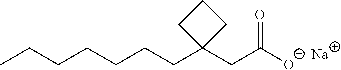 | >63 | 21 | >63 |

The peroxisome proliferator-activated receptors (PPARs), PPARα, PPARδ, and PPARγ are ligand-dependent transcription factors that control expression of several key metabolism-associated genes. The transcriptional activity of representative compounds of formula I to these receptors was assessed using a cell-based GAL4 transactivation assay in HEK293 cells transfected with either PPARα, PPARδ, or PPARγ ligand binding domain (LBD), and was compared to that of the full control agonists GW7647 (PPARα), GW0742 (PPARδ), and rosiglitazone (PPARγ).

Methods

Plasmids: The hinge region and ligand binding domain (LBD) from human PPARα (S167-Y468), PPARδ (S139-Y440) and PPARγ (S176-Y477) were PCR-amplified from a PPARα cDNA clone (cDNA Resource Center, http://www.cdna.org) or from PPARδ1 and PPARγ1 LBD gBlocks™ gene fragments (Integrated DNA Technologies). The PPAR LBD PCR products were inserted in frame with the GAL4 DNA binding domain in the pFN26A(BIND)-hRlu-neo Flexi vector (Promega) at SgfI and PmeI sites to generate GAL4-PPAR-Rluc.

Cell-based PPAR transactivation assay. HEK293 cells were co-transfected with pGL4.35[luc2P/9XGAL4UAS/Hygro] (Promega) and GAL4-PPAR-Rluc plasmids, and after 24 h of incubation were treated with compounds for 24 h. Luciferase activity was determined with the Dual Glo™ luciferase assay (Promega). Firefly luminescence was normalized to the constitutively expressed Renilla luminescence, and results expressed as fold induction of vehicle control or percentage of reference agonist maximal activity.

Results

The results are reported in Table 6.

TABLE 6
Transcriptional activity of the compounds to PPARs

| | PPARα | PPARδ | PPARγ |
|---|---|---|---|
| | (GAL4 reporter) | (GAL4 reporter) | (GAL4 reporter) |
| | % efficacity | % efficacity | % efficacity |
| | relative to | relative to | relative to |
| Compounds | GW7647 | GW0742 | rosiglitazone |
| 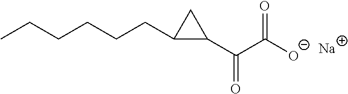 | 34 | −3 | 2 |
| 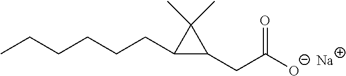 | 91 | 2 | 8 |
| 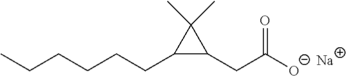 | 33 | −6 | 4 |
| 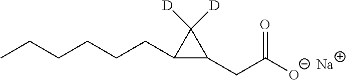 | 20 | 2 | 3 |
| 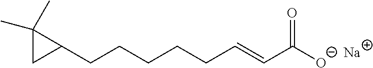 | 10 | 30 | 1 |
| 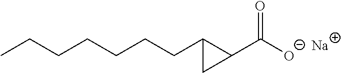 | 90 | 4 | 16 |
| 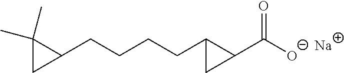 | 43 | 5 | 5 |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A compound of formula (I) or a salt thereof: 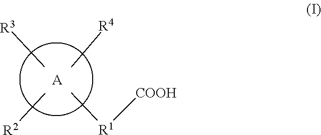
wherein:
A represents a 3- to 6-membered cycloalkane or heterocycloalkane, wherein the cycloalkane or heterocycloalkane are optionally bridged,
R¹ represents a covalent bond or an alkylene or alkenylene chain, wherein the alkylene or alkenylene chain is optionally substituted with ═O,
R² represents a hydrogen atom or an alkyl or alkenyl chain, wherein:
the alkyl or alkenyl chain is optionally substituted with a hydroxy group, or
the alkyl or alkenyl chain is optionally terminated with a carboxyl group or with a 3- to 6-membered cycloalkyl, heterocycloalkyl, aryl or heteroaryl, and
the cycloalkyl, heterocycloalkyl, aryl, and heteroaryl are optimally substituted with one or more alkyl groups, and
R³ and R⁴ are identical to each other or different, are both attached to a same ring atom of A, and represent hydrogen atoms, deuterium atoms, halogen atoms, or methyl groups, or
R³ represents R², wherein R² is as defined above, and R⁴ represents a hydrogen atom,
R¹ and R² are attached on a same ring atom of A or on different ring atoms of A,
wherein the atom of R¹, or of A if R¹ is a covalent bond, that bears the —COOH group is optionally substituted with a second —COOH group,
wherein A, R¹ and R² are such that the shortest continuous chain of carbon atoms and, if present, heteroatoms linking:
the carbon atom or ring heteroatom in R² that is farthest from R¹ or, if R² represents a hydrogen atom, the ring carbon atom or ring heteroatom in A that is farthest from R¹;
to the carbon atom of the COOH group terminating R¹
is 9 to 11 atoms long,
wherein the COOH group may be replaced by an isostere thereof;
and wherein the compound is not 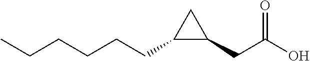
(cascarillic acid) or 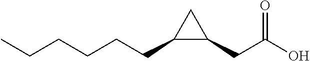
(cis-2-(2-hexylcyclopropyl)-acetic acid).

2. The compound or salt thereof according to claim 1, wherein A represents a 3- to 6-membered cycloalkane.

3-8. (canceled)

9. The compound or salt thereof according to claim 1, wherein A represents:
cyclopropane with R¹ and R² attached on a same atom of the cyclopropane,
cyclopropane with R¹ and R² attached on adjacent atoms of the cyclopropane,
ethylene oxide with R¹ and R² attached on adjacent ring atoms of the ethylene oxide,
cyclobutane with R¹ and R² attached on a same ring atom of the cyclobutane,
cyclobutane with R¹ and R² attached on adjacent ring atoms of the cyclobutane,
cyclobutane with R¹ and R² attached on opposite ring atoms of the cyclobutane,
cyclohexane with R¹ and R² attached on opposite ring atoms of the cyclohexane,
cyclohexane with R¹ and R² attached on ring atoms of the cyclohexane that are separated by a single other ring atom,
piperidine with R¹ and R² attached on opposite ring atoms of the piperidine,
piperazine with R¹ and R² attached on ring atoms of the piperazine that are separated by a single other ring atom, or
bicyclo[2.2.2]octane with R¹ and R² attached on opposite ring atoms of the bicyclo[2.2.2]octane.

10-28. (canceled)

29. The compound or salt thereof according to claim 1, wherein the compound or salt thereof is one of the compounds depicted in Table 1, or a salt thereof:
TABLE 1

| Cmpd # | Structure |
|---|---|
| I | 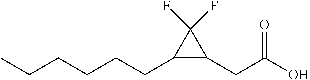 |
| II | 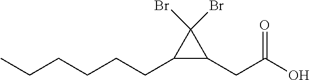 |
| III | 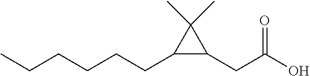 |
| IV | 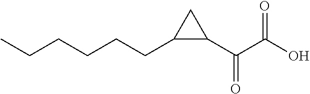 |
| V | 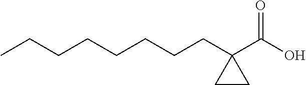 |
| VI | 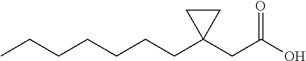 |
| VII | 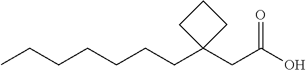 |
| VIII | 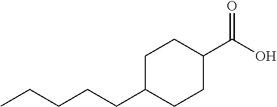 |
| IX | 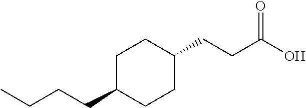 |
| X | 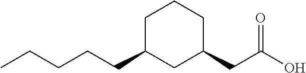 |
| XI | 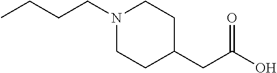 |
| XII | 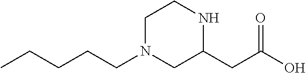 |
| XIII | 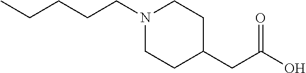 |
| XIV | 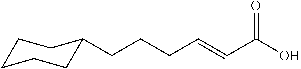 [SMILES: O=C(O)/C=C/CCCC1CCCCC1] |
| XV | 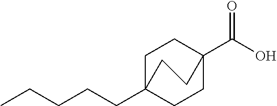 [SMILES: CCCCCC12CCC(C(=O)O)(CC1)CC2] |
| XVI | 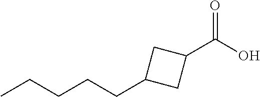 |
| XVII | 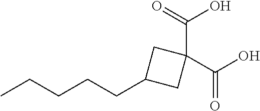 |
| XVIII | 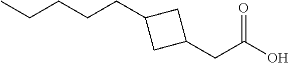 |
| XIX | 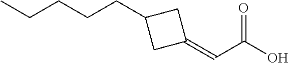 |
| XX | 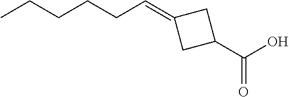 |
| XXI | 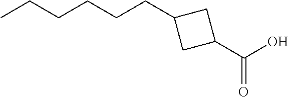 |
| XXII | 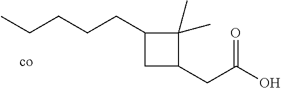 |
| XXIII | 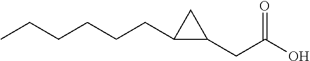 |
| XXIV | 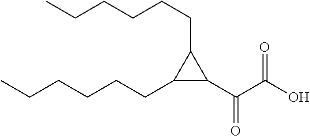 [SMILES: CCCCCCC1C(CCCCCC)C1C(=O)C(=O)O] |
| XXV | 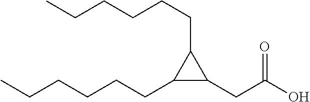 [SMILES: CCCCCCC1C(CCCCCC)C1CC(=O)O] |
| XXVI | 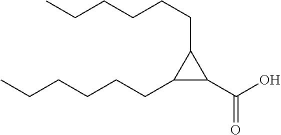 [SMILES: CCCCCCC1C(CCCCCC)C1C(=O)O] |
| XXVII | 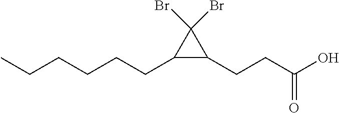 [SMILES: CCCCCCC1C(CCC(=O)O)C1(Br)Br] |
| XXVIII | 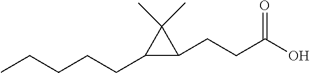 |
| XXIX | 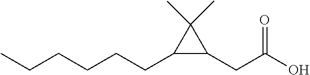 |
| XXX | 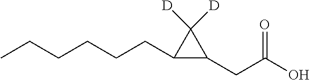 |
| XXXI | 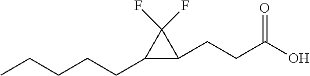 |
| XXXII | 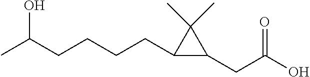 [SMILES: CC(O)CCCCC1C(CC(=O)O)C1(C)C] |
| XXXIII | 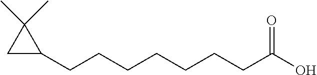 |
| XXXIV | 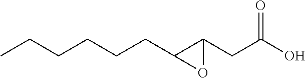 |
| XXXV | 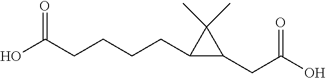 [SMILES: CC1(C)C(CCCCC(=O)O)C1CC(=O)O] |
| XXXVI | 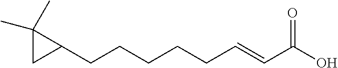 |
| XXXVII | 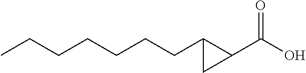 |
| XXXVIII | 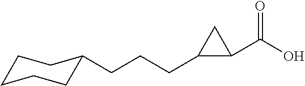 [SMILES: O=C(O)C1CC1CCCC1CCCCC1] |
| XXXIX | 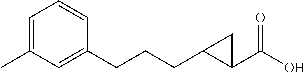 [SMILES: Cc1cccc(CCCC2CC2C(=O)O)c1] |
| XL | 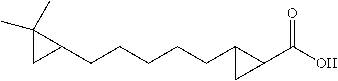 [SMILES: CC1(C)CC1CCCCCC1CC1C(=O)O] |
| XLI | 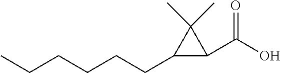 |
| XLII | 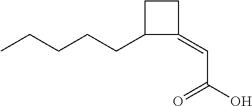 |
| XLIII | 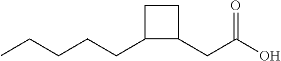 |
| XLIV | 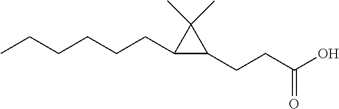 [SMILES: CCCCCCC1C(CCC(=O)O)C1(C)C] |
| XLV | 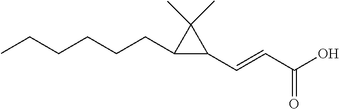 [SMILES: CCCCCCC1C(/C=C/C(=O)O)C1(C)C] |

30. (canceled)

31. The compound or salt thereof according to claim 1, which is a metal salt of the compound.

32. (canceled)

33. The compound or salt thereof according to claim 1, which is a hydrochloride salt of the compound.

34. The compound or salt thereof according to claim 1, which is one of the salts depicted in Table 2:
TABLE 2

| Salt of Cmpd # | Structure |
|---|---|
| I | 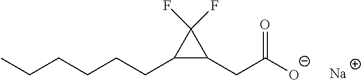 |
| II | 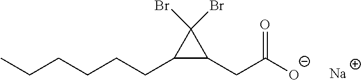 |
| III | 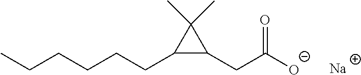 |
| IV | 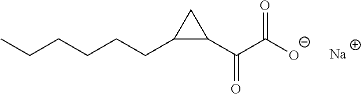 |
| V | 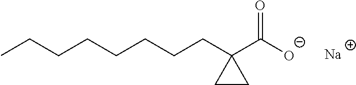 |
| VI | 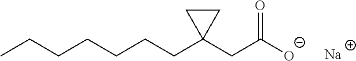 |
| VII | 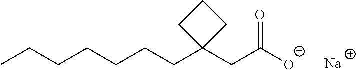 |
| VIII | 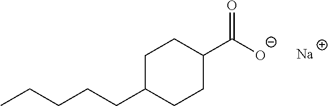 |
| IX | 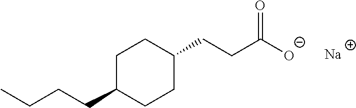 |
| X | 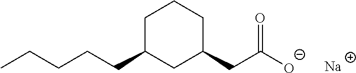 |
| XI | 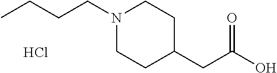 |
| XII | 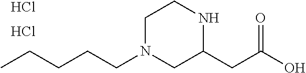 |
| XIII | 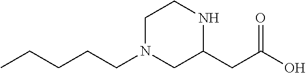 |
| XIV | 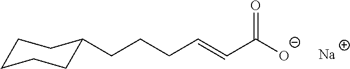 [SMILES: O=C([O-])/C=C/CCCC1CCCCC1 ; [Na+]] |
| XV | 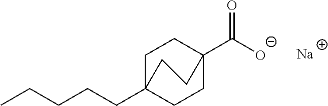 [SMILES: CCCCCC12CCC(C(=O)[O-])(CC1)CC2 ; [Na+]] |
| XVI | 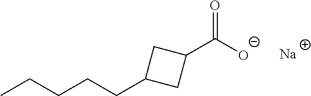 |
| XVII | 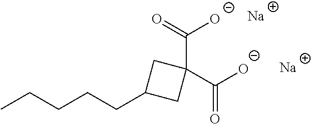 |
| XVIII | 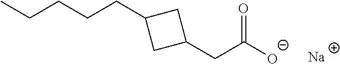 |
| XIX | 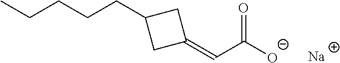 |
| XX | 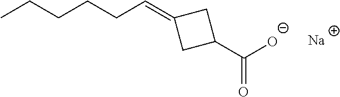 |
| XXI | 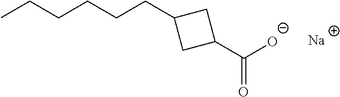 |
| XXII | 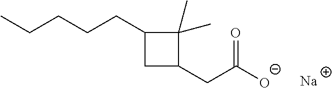 |
| XXIII | 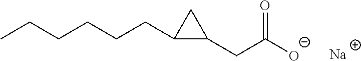 |
| XXIV | 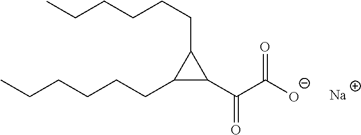 [SMILES: CCCCCCC1C(CCCCCC)C1C(=O)C(=O)[O-] ; [Na+]] |
| XXV | 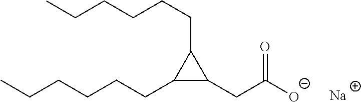 [SMILES: CCCCCCC1C(CCCCCC)C1CC(=O)[O-] ; [Na+]] |
| XXVI | 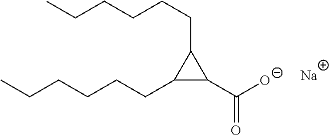 [SMILES: CCCCCCC1C(CCCCCC)C1C(=O)[O-] ; [Na+]] |
| XXVII | 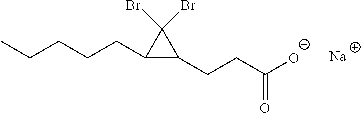 |
| XXVIII | 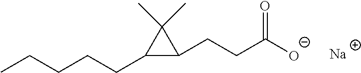 |
| XXIX | 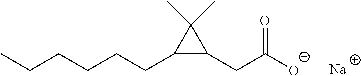 |
| XXX | 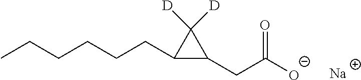 |
| XXXI | 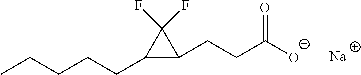 |
| XXXII | 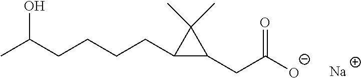 [SMILES: CC(O)CCCCC1C(CC(=O)[O-])C1(C)C ; [Na+]] |
| XXXIII | 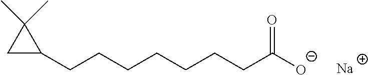 |
| XXXIV | 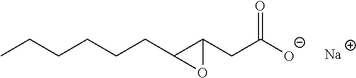 |
| XXXV | 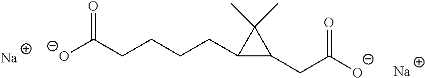 [SMILES: [Na+] ; CC1(C)C(CCCCC(=O)[O-])C1CC(=O)[O-] ; [Na+]] |
| XXXVI | 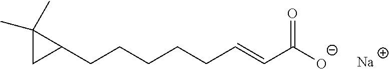 |
| XXXVII | 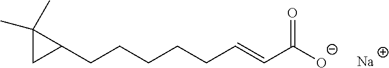 |
| XXXVIII | 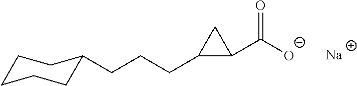 [SMILES: O=C([O-])C1CC1CCCC1CCCCC1 ; [Na+]] |
| XXXIX | 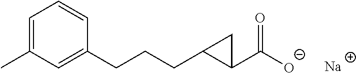 [SMILES: Cc1cccc(CCCC2CC2C(=O)[O-])c1 ; [Na+]] |
| XL | 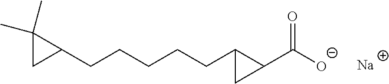 [SMILES: CC1(C)CC1CCCCCC1CC1C(=O)[O-] ; [Na+]] |
| XLI | 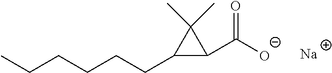 |
| XLII | 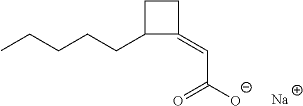 |
| XLIII | 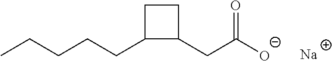 |
| XLIV | 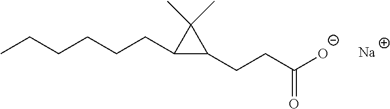 [SMILES: CCCCCCC1C(CCC(=O)[O-])C1(C)C ; [Na+]] |
| XLV | 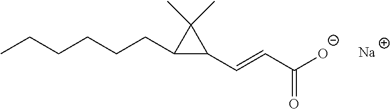 [SMILES: CCCCCCC1C(/C=C/C(=O)[O-])C1(C)C ; [Na+]] |

35. (canceled)

36. A composition comprising the compound or salt thereof according to claim 1 and a carrier or excipient.

37. A method for stimulating hematopoiesis or erythropoiesis in a subject in need thereof comprising administering to the subject an effective amount of the compound or salt thereof according to claim 1.

38. A method for treating anemia or leukopenia in a subject in need thereof comprising administering to the subject an effective amount of the compound or salt thereof according to claim 1.

39. The method of claim 38, wherein the leukopenia and/or anemia is caused by chemotherapy.

40. The method of claim 38, wherein the leukopenia and/or anemia is caused by bone marrow transplantation.

41. The method of claim 37, wherein the subject suffers from immunodeficiency.

42. A method for preventing and/or treating fibrosis in a subject in need thereof comprising administering to the subject an effective amount of the compound or salt thereof according to claim 1.

43. The method of claim 42, wherein the fibrosis is kidney fibrosis, lung fibrosis, liver fibrosis, heart fibrosis, bone marrow fibrosis or skin fibrosis.

44. A method for treating cancer in a subject in need thereof comprising administering to the subject an effective amount of the compound or salt thereof according to claim 1.

45. A method for treating hypertension in a subject in need thereof comprising administering to the subject an effective amount of the compound or salt thereof according to claim 1.

46. A method for treating a metabolic condition in a subject in need thereof comprising administering an effective amount of the compound or salt thereof according to claim 1.

47. The method of claim 46, wherein the metabolic condition is metabolic syndrome, pre-diabetes, or diabetes.

48. The method of claim 46, wherein the diabetes is Type II diabetes.

49-78. (canceled)
